(19) United States
(12) Patent Application Publication
(10) Pub. No.: US 2013/0347138 A1
(43) Pub. Date: Dec. 26, 2013

(54) Cells and Vertebrates for Enhanced Somatic Hypermutation and Class Switch Recombination

(71) Applicant: Kymab Limited, (US)

(72) Inventor: E-Chiang Lee, Cambridge (GB)

(73) Assignee: Kymab Limited, Cambridge (GB)

(21) Appl. No.: 13/890,147

(22) Filed: May 8, 2013

Related U.S. Application Data
(63) Continuation of application No. PCT/GB2011/052156, filed on Nov. 7, 2011.

(30) Foreign Application Priority Data
Nov. 8, 2010 (GB) 1018786.2
Dec. 3, 2010 (GB) 1020483.2

(51) Int. Cl. A01K 67/027

(52) U.S. Cl. CPC A01K 67/0278
USPC 800/18; 800/14; 435/326; 435/328; 530/387.3; 530/350; 424/133.1

(57) ABSTRACT

The invention provides improved non-human vertebrates and non-vertebrate cells capable of expressing antibodies, eg, comprising human variable region sequences. The invention provides for enhanced AID and/or AID homologue spectra, thereby providing for the increased diversity as a result of somatic hypermutation and/or class-switch recombination during in vivo antibody generation. The invention also provides methods of generating antibodies using such vertebrates, as well as the antibodies per se, therapeutic compositions thereof and uses.

This application is a continuation of international Application PCT/GB2011/052156, filed 7 Nov. 2011, which claims the benefit of GB1018786.2 filed 8 Nov. 2010, and of GB1020483.2 filed 3 Dec. 2010. Each of these applications is herein incorporated by reference in their entirety.

The present invention relates inter alia to non-human vertebrates or vertebrate cells whose genomes comprise antibody variable domain gene segments which are expressible in the context of improved intracellular machinery for somatic hypermutation (SHM) and class switch recombination (CSR). Specifically, the invention involves the enhancement of the spectrum of activity of AID/APOBEC enzyme family members, which enzymes create diversity in immunoglobulin sequences by SHM and CSR. The invention also relates to such vertebrates and cells which are transgenic mice or rats or transgenic mouse or rat cells. Furthermore, the invention relates to a method of using the vertebrates to isolate antibodies or nucleotide sequences encoding antibodies. Antibodies, nucleotide sequences, pharmaceutical compositions and uses are also provided by the invention.

### BACKGROUND

The AID/APOBEC Family

The AID/APOBEC family is a family of RNA or DNA editing enzymes that mediate the deamination of cytosine to uracil in nucleic acid sequences (see, eg, Conticello, Genome Biol. 2008; 9(6):229. Epub 2008 Jun. 17. Review; Conticello et al, Mol Biol Evol, 22:367-377 (2005); and U.S. Pat. No. 6,815,194). See also FIG. 8 of WO2010/113039, which publication including FIG. 8 are explicitly incorporated herein by reference. This includes incorporation herein of all AID/APOBEC family member sequences disclosed in WO2010/113039, as though explicitly written herein for use in the present invention and for possible inclusion in claims below.

AID=“activation-induced cytidine deaminase”. Alternative names are: AICDA, HIGM2, CDA2 and ARP2, APOBEC=“apolipoprotein B mRNA editing enzyme, catalytic polypeptide-like”. The nucleotide and amino acid sequences of human, mouse and rat APOBECs are disclosed by reference to table 2 below.

Members of the AID/APOBEC family include:

APOBEC1

APOBEC2

APOBEC3A

APOBEC3C

APOBEC3D (aka “APOBEC3E”)

APOBEC3F

APOBEC3G

APOBEC3H

APOBEC4

Reference is made to Jarmuz et al, Genomics. 2002 March; 79(3):285-96.

EP1174509 discloses AID sequences. WO03/061363 discloses the expression of AID in cells. WO03/095636 discloses the expression of AID or AID homologues in cells, in order to confer a mutator phenotype. WO2005/023865 discloses methods for generating diversity in immunoglobulin genes using AID. WO2006/053021 discloses methods for engineering variant polypeptides using AID expressed in a cell. WO2008/103475 discloses the design of synthetic genes to increase or decrease hot- and cold-spots for SHM. WO2010/113039 discloses mutants of AID. Reference is also made to “AID upmutants isolated using a high-throughput screen highlight the immunity/cancer balance limiting DNA deaminase activity”; Wang M, Yang Z, Rada C, Neuberger M S; Nat Struct Mol Biol. 2009 July; 16(7):769-76; and “Altering the spectrum of immunoglobulin V gene somatic hypermutation by modifying the active site of AID”; Wang M, Rada C, Neuberger M S; J Exp Med. 2010 Jan. 18; 207(1): 141-53.

### SUMMARY OF THE INVENTION

A first configuration of the present invention provides, in a first aspect, a transgenic non-human vertebrate or vertebrate cell whose genome comprises

(a) a transgene, wherein the transgene comprises at least one (optionally unrearranged) human V region, at least one human J region, and optionally at least one human D region, wherein said regions are upstream of a constant region;

(b) a first expressible gene encoding a first activation-induced deaminase (AID) or an AID homologue; and

(c) a second expressible gene encoding a second AID or an AID homologue, wherein the first and second AIDs are not identical;

optionally wherein the transgene comprises a rearranged VDJ or VJ nucleotide sequence (e.g.,(e.g., a rearranged VDJ or VJ nucleoside sequence comprising human variable region sequences).

An aspect provides transgenic mouse or mouse cell according to the first configuration of the invention, comprising

(a) a transgene, wherein the transgene comprises substantially the full human repertoire of IgH V, D and J regions, wherein said regions are upstream of a constant region, wherein the constant region is a mouse constant region or derived from a mouse constant region, optionally comprising a mouse Sμ switch and optionally a mouse Cμ region;

(b) a first expressible gene encoding a first activation-induced deaminase (AID) or an AID homologue; and

(c) a second expressible gene encoding a second AID or an AID homologue, wherein the first and second AIDs or AID homologues are not identical.

An aspect provides transgenic rat or rat cell according to the first configuration of the invention, comprising

(a) a transgene, wherein the transgene comprises substantially the full human repertoire of IgH V, D and J regions, wherein said regions are upstream of a constant region, wherein the constant region is a rat constant region or derived from a rat constant region, optionally comprising a rat Sμ switch and optionally a rat Cμ, region;

(b) a first expressible gene encoding a first activation-induced deaminase (AID) or an AID homologue; and

(c) a second expressible gene encoding a second AID or an AID homologue, wherein the first and second AIDs or AID homologues are not identical.

An alternative aspect of the first configuration of the invention provides:—

A transgenic non-human vertebrate or vertebrate cell whose genome comprises

(a′) at least one immunoglobulin V region, at least one immunoglobulin J region, and optionally at least one immunoglobulin D region (optionally a rearranged VDJ or VJ nucleotide sequence), wherein said regions are upstream of a constant region;

(b) a first expressible gene encoding a first activation-induced deaminase (AID) or an AID homologue; and

(c) a second expressible gene encoding a second AID or an AID homologue, wherein the first and second AIDs are not identical.

Features described herein with reference to the first aspect of the first configuration of the invention, are also to be read as applying mutatis mutandis to the alternative aspect described above, and as such this provides a basis for inclusion of any such features in combination with the alternative aspect in the claims.

In this aspect, in one embodiment, the first and second AIDs or homologues are derived from (or wild-type versions from) moderately divergent species, as described below. This provides the advantage of harnessing AID's that have evolved in nature in a way that increases the spectrum of diversity, which brings benefits as discussed below. For example, where the vertebrate in this alternative aspect is a mouse, the first AID is a wild-type AID from a divergent species (e.g.,(e.g., chicken or Xenopus) or a homologue thereof, and the second AID is mouse AID (e.g.,(e.g., AID endogenous to said mouse). In another example, the vertebrate in this alternative aspect is a rat, the first AID is a wild-type AID from a divergent species (e.g.,(e.g., chicken or Xenopus) or a homologue thereof, and the second AID is rat AID (e.g.,(e.g., AID endogenous to said rat).

The vertebrate or cell of any preceding aspect is provided, wherein the first AID or AID homologue gene is the wild-type AID gene.

The vertebrate or cell of any preceding aspect is provided, wherein the second AID or AID homologue gene comprises the nucleotide sequence of human AID (SEQ ID NO: 1), human APOBEC1, human APOBEC3C, human APOBEC3F, human APOBEC3G, or a functional mutant that is at least 95, 96, 97, 98 or 99% identical thereto.

In an aspect of the first configuration, the first AID or AID homologue gene is the wild-type AID gene; optionally wherein

(i) the vertebrate is a mouse or a rat, or the vertebrate cell is a mouse cell or a rat cell and the first expressible gene encodes a human AID and the second expressible gene encodes a chicken AID; or

(ii) the vertebrate is a mouse or a rat, or the vertebrate cell is a mouse cell or a rat cell and the first expressible gene encodes a human AID and the second expressible gene encodes an African clawed frog AID; or

(iii) the vertebrate is a mouse or a rat, or the vertebrate cell is a mouse cell or a rat cell and the first expressible gene encodes a human AID and the second expressible gene encodes mouse AID (e.g.,(e.g., AID endogenous to said mouse when said vertebrate is a mouse or vertebrate cell is a mouse cell); or

(iv) the vertebrate is a mouse or a rat, or the vertebrate cell is a mouse cell or a rat cell and the first expressible gene encodes a human AID and the second expressible gene encodes rat AID (e.g.,(e.g., AID endogenous to said rat when said vertebrate is a rat or vertebrate cell is a rat cell). This has benefits of expanding the AID or AID homologue spectrum so that the design is provided to enhance antibody sequence diversity subsequent selection after immunisation.

A second configuration of the invention, in a first aspect, provides a transgenic non-human vertebrate or vertebrate cell whose genome comprises

(a) a transgene, wherein the transgene comprises at least one (optionally unrearranged) human V region, at least one human J region, and optionally at least one human D region, wherein said regions are upstream of a constant region;

(b) a first expressible gene encoding a first activation-induced deaminase (AID) or an AID homologue; and

(c) a second expressible gene encoding a second AID or an AID homologue,

wherein each AID or AID homologue is a human AID or AID homologue, or a functional mutant thereof; and

optionally wherein the transgene instead comprises a rearranged VDJ or VJ nucleotide sequence (e.g.,(e.g., a rearranged VDJ or VJ nucleotide sequence comprising human variable region sequences); and

optionally wherein the first and second AIDs or homologues are not identical.

An aspect of the second configuration provides a transgenic mouse or mouse cell, comprising

(a) a transgene, wherein the transgene comprises substantially the full human repertoire of IgH V, D and J regions, wherein said regions are upstream of a constant region, wherein the constant region is a mouse constant region or derived from a mouse constant region, optionally comprising a mouse Sμ switch and optionally a mouse Cμ region;

(b) a first expressible gene encoding a first activation-induced deaminase (AID) or an AID homologue; and

(c) a second expressible gene encoding a second AID or an AID homologue,

wherein each AID or AID homologue is a human AID or AID homologue, or a functional mutant thereof.

An aspect of the second configuration provides a transgenic rat or rat cell, comprising

(a) a transgene, wherein the transgene comprises substantially the full human repertoire of IgH V, D and J regions, wherein said regions are upstream of a constant region, wherein the constant region is a rat constant region or derived from a rat constant region, optionally comprising a rat Sμ switch and optionally a rat Cμ region;

(b) a first expressible gene encoding a first activation-induced deaminase (AID) or an AID homologue; and

(c) a second expressible gene encoding a second AID or an AID homologue,

wherein each AID or AID homologue is a human AID or AID homologue, or a functional mutant thereof.

An alternative aspect of the second configuration of the invention provides:—

A transgenic non-human vertebrate or vertebrate cell whose genome comprises

(a′) at least one immunoglobulin V region, at least one immunoglobulin J region, and optionally at least one immunoglobulin D region (optionally a rearranged VDJ or VJ nucleotide sequence), wherein said regions are upstream of a constant region;

(b) a first expressible gene encoding a first activation-induced deaminase (AID) or an AID homologue; and

(c) a second expressible gene encoding a second AID or an AID homologue,

wherein each AID or AID homologue is a human AID or AID homologue, or a functional mutant thereof; and

optionally wherein the first and second AIDs or homologues are not identical.

Features described herein with reference to the first aspect of the second configuration of the invention, are also to be read as applying mutatis mutandis to the alternative aspect of the second configuration described above, and as such this provides a basis for inclusion of any such features in combination with this alternative aspect in the claims.

In an aspect of the first or second configuration,

(i) the transgene comprises at least one human IgH V region, at least one human J region, and optionally at least one human D region; and

(ii) the vertebrate or cell comprises a further transgene, the further transgene comprising at least one human IGλ V region and at least one human J region.

In an aspect of the first or second configuration,

(i) the transgene comprises at least one human IgH V region, at least one human J region, and optionally at least one human D region; and

(ii) the vertebrate or cell comprises a further transgene, the further transgene comprising at least one human Igλ V region and at least one human J region.

In an aspect of the first or second configuration,

(i) the transgene comprises substantially the full human repertoire of IgH V, D and J regions; and

(ii) the vertebrate or cell comprises substantially the full human repertoire of Igκ V and J regions and/or substantially the full human repertoire of Igλ V and J regions.

In the vertebrate or cell of any configuration, the expression of at least one of the AIDs or AID homologues is inducible.

In the vertebrate or cell of any configuration, the AID homologue(s) and/or AID mutant(s) are present in the genome under operable control of wild-type AID gene control elements, e.g., control elements that are endogenous to the vertebrate or vertebrate cell.

In the vertebrate or cell of any configuration, at least one V, D and/or J region sequence in the transgene has been codon-optimised for AID or an AID homologue, optionally wherein the V, D and/or J sequence has been changed to include a sequence motif selected from the group consisting of DGYW, WRC, WRCY, WRCH, RGYW, AGY,TAC, WGCW, wherein W=A or T, Y=C or T, D=A, G or T, H=A or C or T, and R=A or G.

In the vertebrate or cell of any configuration, in one embodiment the genome comprises a third expressible gene encoding a third AID or AID homologue. Thus, there are provided at least three expressible AID or homologue genes in the genome which provides the advantage of potentially enhanced levels of AID in the vertebrate or cell. Good levels of AID are desirable to provide for enhanced SHM and/or CSR and to maximise the spectrum of mutations. In one example, the vertebrate is a mouse or the vertebrate cell is a mouse cell, wherein the first expressible gene encodes a non-endogenous AID or AID homologue (e.g.,(e.g., one from a moderately divergent species as herein defined) and the second and third expressible genes are wild-type AID genes endogenous to the mouse or mouse cell. In another example, the vertebrate is a rat or the vertebrate cell is a rat cell, wherein the first expressible gene encodes a non-endogenous AID or AID homologue (e.g.,(e.g., one from a moderately divergent species as herein defined) and the second and third expressible genes are wild-type AID genes endogenous to the rat or rat cell. In a further embodiment, the vertebrate or cell comprises a fourth expressible gene encoding AID or a homologue, eg, where this is a second copy of the third expressible gene.

The invention provides a B-cell, hybridoma or a stem cell, optionally an embryonic stem cell or haematopoietic stem cell, according to any configuration or aspect of the invention.

The invention provides a method of isolating an antibody or nucleotide sequence encoding said antibody, the method comprising

(a) immunising a vertebrate according to any configuration or aspect of the invention with an antigen such that the vertebrate produces antibodies; and

(b) isolating from the vertebrate an antibody that specifically binds to said antigen and/or a nucleotide sequence encoding at least the heavy and/or the light chain variable regions of said antibody;

optionally wherein the variable regions of said antibody are subsequently joined to a human constant region.

The invention provides an antibody produced by the method of the invention, optionally for use in medicine.

The invention provides a nucleotide sequence encoding the antibody of the invention, optionally wherein the nucleotide sequence is part of a vector.

The invention provides a pharmaceutical composition comprising the antibody of the invention and a diluent, excipient or carrier.

The invention provides the use of the antibody of the invention in the manufacture of a medicament for the treatment and/or prophylaxis of a disease or condition in a patient, eg, a human.

The invention provides a chimaeric AID comprising a mouse or rat AID in which the active-site loop has been replaced with a foreign active-site loop, optionally a human, chicken, bird, fish, reptile, Xenopus, catfish or zebrafish AID active-site loop.

The invention provides a nucleic acid comprising a nucleotide sequence encoding the chimaeric AID of the invention.

The invention provides a nucleic acid comprising a nucleotide sequence encoding a chimaeric AID, wherein the nucleotide sequence comprises a nucleotide sequence encoding mouse or rat AID wherein exon 3 has been replaced with an exon 3 nucleotide sequence selected from a human, chicken, bird, fish, reptile, Xenopus, catfish or zebrafish AID gene exon 3 nucleotide sequence.

The invention provides a nucleic acid comprising a nucleotide sequence encoding a chimaeric AID, wherein the nucleotide sequence comprises a nucleotide sequence encoding mouse or rat AID wherein the active-site loop-encoding nucleotide sequence has been replaced with an active-site loop-encoding nucleotide sequence selected from a human, chicken, bird, fish, reptile, Xenopus, catfish or zebrafish AID active-site loop-encoding nucleotide sequence.

The invention provides a chimaeric AID comprising an amino acid sequence selected from the group consisting of SEQ ID NO: 54, 56 and 58, or a sequence that is at least 80% identical thereto.

The invention provides a nucleic acid comprising a nucleotide sequence encoding a chimaeric AID, wherein the nucleotide sequence is selected from the group consisting of SEQ ID NO: 53, 55 and 57, or a sequence that is at least 80% identical thereto.

The invention provides a nucleotide sequence encoding a chimaeric AID of the invention when integrated into the genome of a non-human vertebrate mammal or the genome of a non-human vertebrate cell, optionally wherein said genome further comprises an endogenous gene encoding a wild-type AID or a gene encoding an AID, chimaeric AID or an AID homologue.

The invention addresses the desirability to design a non-human vertebrate or cell to enhance sequence diversity resulting from SHM and/or CSR. This then provides for the potential of a greater antibody sequence space for in vivo selection of antibodies against target antigens with which the vertebrate is subsequently immunised (said vertebrate being a vertebrate of the invention optionally produced using a cell of the invention). To this end, however, the invention does not rely on increasing diversity by increasing enzymatic efficiency of AID or AID homologues (which can be relatively difficult to control and can cause undesirable chromosome translocations sometimes implicated in tumour formation (see, for example, R Maul & P Gearhart, Advances in Immunology, 2010, volume 105, Chapter 6 (pp 159-191): AID and Somatic Hypermutation). Rather, diversity resulting from SHM and CSR is addressed by the present invention in all its configurations by extending the spectrum of AID or AID homologue activity. This can be managed by the choice of AIDs or AID homologues to be expressed by the vertebrate or vertebrate cell, according to the invention. The use in the present invention of non-identical AIDs or AID homologues provides for greater AID or AID homologue diversity in SHM and CSR activity spectra (and thus a resultant design for improved antibody diversity upon immunisation) in the vertebrate or vertebrate cell of the invention, compared to the retention only of homozygous copies of AID or AID homologue that is endogenous to the vertebrate. In addition, the use of one or more human AIDs or AID homologues is advantageous in the context of transgenes that comprise human V, D and/or J sequences, since these provide substrates on which AID can act in SHM and CSR. Again, such a design is provided to enhance sequence and antibody diversity by exploiting a desirable spectrum of AID or AID homologue activity.

Reference is made to “Evolution of Ig DNA sequence to target specific base positions within codons for somatic hypermutation”, Shapiro et al, J Immunol. 2002 Mar. 1; 168(5):2302-6; and “The nucleotide targets of somatic mutation and the role of selection in immunoglobulin heavy chains of a teleost fish”, Yang F et al, J Immunol. 2006 Feb. 1; 176(3):1655-67, which describe studies into the relative preference for codon usage (mutability index) amongst AIDs from different species. Codon preference is shown to be different amongst AID from different species. Comparison of the trinucleotide mutability index of the immunoglobulin loci from variety of species suggests different mutational spectra of AIDs.

### BRIEF DESCRIPTION OF THE FIGURES

FIG. 1: A phylogenetic tree of AIDs from various non-human vertebrate species; and

FIG. 2: Alignment of AID amino acid sequences from various non-human vertebrate species.

FIG. 3: Alignment of AID amino acid sequences from various non-human vertebrate species showing exon boundaries, position of catalytic: residues and active-site loops. Exon 3: a.a. residues 53-143 of human, rat or mouse AID sequence; Active-site loop: a.a residues 113-120 of human, rat or mouse AID sequence.

### DETAILED DESCRIPTION OF THE INVENTION

All nucleotide coordinates for the mouse are from NCBI m37, April 2007 ENSEMBL Release 55.37h for the mouse C57BL/6J strain. Human nucleotides are from GRCh37, February 2009 ENSEMBL Release 55.37 and rat from RGSC 3.4 December 2004 ENSEMBL release 55.34w.

In a first configuration, the invention provides a transgenic non-human vertebrate or vertebrate cell whose genome comprises

(a) a transgene, wherein the transgene comprises at least one human V region, at least one human J region, and optionally at least one human D region, wherein said regions are upstream of a constant region;

(b) a first expressible gene encoding a first activation-induced deaminase (AID) or an AID homologue; and

(c) a second expressible gene encoding a second AID or an AID homologue, wherein the first and second AIDs are not identical;

optionally wherein instead the transgene comprises a rearranged VDJ or VJ nucleotide sequence.

The inserted human genes may be derived from the same individual or different individuals, or be synthetic or represent human consensus sequences.

Although the number of V D and J regions is variable between human individuals, in one aspect there are considered to be 51 human V genes, 27 D and 6 J genes on the heavy chain, 40 human V genes and 5 J genes on the kappa light chain and 29 human V genes and 4 J genes on the lambda light chain (Janeway and Travers, Immunobiology, Third edition)

The rearranged VDJ and VJ sequences discussed herein (in the context of any configuration of the invention) can be VDJ or VJ sequences encoding the variable region of a pre-existing antibody that binds a predetermined antigen, eg, an antibody selected from the group consisting of abagovomab, abciximab, adalimumab, adecatumumab, afelimomab, afutuzumab, alacizumab, ALD518, alemtuzumab, altumomab, anatumomab, anrukinzumab, apolizumab, arcitumomab, aselizumab, atiizumab, atorolimumab, bapineuzumab, basiliximab, bavituximab, bectumomab, belimumab, benrallzumab, bertilimumab, besilesomab, bevacizumab, biciromab, bivatuzumab, blinatumomab, brentuximab, briakinumab, canakinumab, cantuzumab, capromab, catumaxomab, CC49, cedelizumab, certolizumab, cetuximab, citatuzumab, cixutumumab, clenoliximab, clivatuzumab, conatumumab, CR6261, dacetuzumab, daclizumab, daratumumab, denosumab, detumomab, dorlimomab, dorlixizumab, ecromeximab, eculizumab, edobacomab, edrecolomab, efalizumab, efungumab, elotuzumab, elsilimomab, enlimomab, epitumomab, epratuzumab, erlizumab, ertumaxomab, etaracizumab, exbivirumab, fanolesomab, faralimomab, farletuzumab, felvizumab, fezakinumab, figitumumab, fontolizumab, foravirumab, fresolimumab, galiximab, gantenerumab, gavilimomab, gemtuzumab, girentuximab, glembatumumab, golimumab, gomiliximab, ibalizumab, ibritumomab, igovomab, imciromab, infliximab, intetumumab, inolimomab, inotuzumab, ipilimumab, iratumumab, keliximab, labetuzumab, lebrikizumab, lemalesomab, lerdelimumab, lexatumumab, libivirumab, lintuzumab, iuoatumumab, lumiliximab, mapatumumab, maslimomab, matuzumab, mepolizumab, metelimumab, milatuzumab, minretumomab, mitumomab, morolimumab, motavizumab, muromonab, nacolomab, naptumomab, natalizumab, nebacumab, necitumumab, nerelimomab, nimotuzumab, nofetumomab, ocreiizumab, odulimomab, ofatumumab, olaratumab, omalizumab, oportuzumab, oregovomab, otelixizumab, pagibaximab, palivizumab, panitumumab, panobacumab, pascolizumab, pemtumomab, pertuzumab, pexelizumab, pintumomab, priliximab, pritumumab, PRO 140, rafivirumab, ramucirumab, ranibizumab, raxibacumab, regavirumab, resllzumab, rilotumumab, rituximab, robatumumab, rontalizumab, rovelizumab, ruplizumab, satumomab, sevirumab, sibrotuzumab, sifalimumab, siltuximab, siplizumab, solanezumab, sonepcizumab, sontuzumab, stamulumab, sulesomab, tacatuzumab, tadocizumab, talizumab, tanezumab, taplitumomab, tefibazumab, telimomab, tenatumomab, teneliximab, teplizumab, TGN1412, ticilimumab, tremelimumab, tigatuzumab, TNX-650, tocilizumab, toralizumab, tositumomab, trastuzumab, tremelimumab, tucotuzumab, tuvirumab, urtoxazumab, ustekinumab, vapaliximab, vedolizumab, veltuzumab, vepalimomab, visilizumab, volociximab, votumumab, zalutumumab, zanolimumab, ziralimumab, zolimomab aritox, 3F8, ReoPro™, Humira™, Campath™, MabCampath™, Hybri-ceaker™, CEA-Scan™, Actemra™, RoActemra™, Simulect™, LymphoScan™, Benlysta™, LymphoStat-B™, Scintimun™, Avastin™, FibriScint™, Maris™, Prostascint™, Removab™, Cimzia™, Erbitux™, Zenapax™, Prolia™, Solids™, Panorex™, Raptiva™, Mycograb™, Rexomun™, Abegrin™, NeutroSpec™, HuZAF™, Mylotarg™, Simponi™, Zevalin™, Indimacis-125™, Myosdnt™, Remicade™, CEA-Cide™, Bosatria™, Numax™, Orthocione OKT3™, Tysabri™, Theracim™, Theraloc™, Verluma™, Arzerra™, Xolair™, OvaRex™, Synagis™, Bbosynagis™, Vectibix™, Theragyn™, Omnitarg™, Lucentis™, MabThera™, Rituxan™, LeukArrest™, Antova™, LeukoScan™, AFP-Cide™, Aurexis™, Actemra™, RoActemra™, Bexxar™, Herceptin™, Stelara™, Nuvion™, HumaSPECT™, HuMax-EGFr™ and HuMax-CD4™.

Optionally, the pre-existing antibody is antibody selected from the group consisting of abciximab, adalimumab, alemtuzumab, basiliximab, belimumab, bevacizumab, cetuximab, certolizumab, daclizumab, denosumab, eculizumab, efalizumab, gemtuzumab, golimumab, ibritumomab, infliximab, muromonab, natalizumab, ofatumumab, omalizumab, palivlzumab, panitumumab, ranibizumab, rituximab, tocilizumab, tositumomab, trastuzumab, BenLysta™, Actemra™, Arzerra™, Prolia™, ReoPro™, Humira™, Campath™, Simulect™, Avastin™, Erbitux™, Cimzia™, Zenapax™, Soliris™, Raptiva™, Mylotarg™, Zevalin™, Remicade™, Orthoclone OKT3™, Tysabri™, Xolair™, Synagis™, Vectibix™, Lucentis™, Rituxan™, Mabthera™, Bexxar™ and Simponi™, eg, the antibody is tocilizumab or Actemra™; or the antibody is belimumab or Benlysta™; or the antibody is panitumumab or Vectibix™.

Techniques for constructing non-human vertebrates and vertebrate cells whose genomes comprise a transgene containing human V, J and optionally D regions are well known in the art. For example, reference is made to co-pending application PCT/GB2010/051122, US7501552, US6673986, U.S. Pat. No. 6,130,364, WO2009/076464 and U.S. Pat. No. 6,586,251, the disclosures of which are incorporated herein by reference in their entirety.

In one embodiment, each AID or AID homologue is a wild-type AID. For example, each AID or AID homologue is selected from a reptile or fish; or human, murine, rat, rabbit, bovine, canine, chicken, porcine, chimpanzee, macaque, horse, Xenopus, pufferfish, catfish (e.g.,(e.g., channel catfish), shark, Camelid (e.g.,(e.g., llama, alpaca or camel), and zebrafish AID or AID homologue (e.g.,(e.g., optionally APOBEC1, APOBEC2, an APOBEC3, APOBEC3A, APOBEC33, APOBEC3C, APOBEC3D, APOBEC3E, APOBEC3F, APOBEC3G, APOBEC3H or APOBEC4), provided that the first and second AIDs or homologues are not identical. Suitable AID sequences are listed in the sequence listing below as SEQ ID NOs: 1 to 11, and also those sequences listed in Tables 1 and 3 below, as well as those disclosed in WO2010/113039 (see SEQ ID NOs: 1 to 14 referenced on page 9 of that publication, these sequences being incorporated herein as though explicitly written herein for use in the present invention and for potential inclusion in claims below). For example, the first AID or AID homologue is endogenous to the vertebrate (or vertebrate from which the cell of the invention is derived) or a functional mutant thereof. Additionally or alternatively to this, in one embodiment the second AID is human AID (nucleotide sequence=SEQ ID NO: 1 in the sequence listing herein; amino acid sequence=SEQ ID NO: 12 in the sequence listing herein; or SEQ ID NO: 1 or 2 disclosed in WO2010/113039) or a functional mutant that is at least 95, 96, 97, 98 or 99% identical thereto or 100% identical thereto.

Advantageously, the first and second AID or AID homologues are wild-type and are moderately divergent. By moderately divergent, it is intended that the species from which the AID or homologues are derived are divergent as indicated by the extent of sequence identity of the enzyme amino acid sequences or as indicated by extent of relatedness in a phylogenetic tree including the AID or homologue species. Moderate identity is an advantageous embodiment in which species are selected that are sufficiently divergent to provide for AID or AID homologue spectrum diversity (and thus a resultant design for improved antibody diversity) when present in the vertebrate or vertebrate cell of the invention, and yet are sufficiently related (albeit moderately distantly, eg, as indicated by a phylogenetic tree or sequence identity) to operate in the context of the transgene and the vertebrate (vertebrate cell) being used.

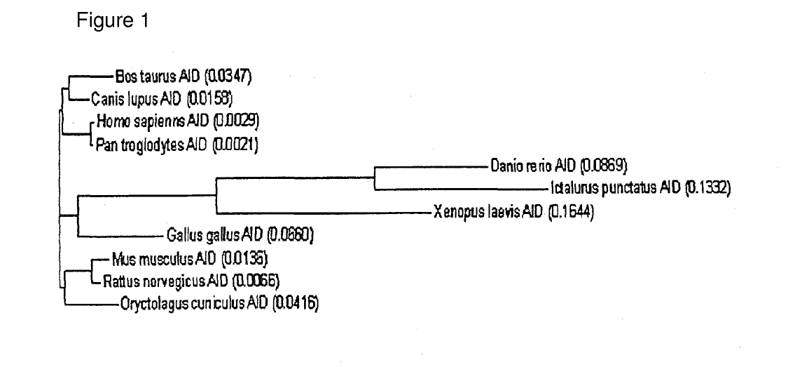

In this respect, reference is made to FIG. 1 which shows a phylogenetic tree. It can be seen that there are, broadly speaking, three divergent groups of AID species: (i) Bos taurus (bovine), Canis lupus (dog). Homo sapiens (human) and Pan troglodytes (chimpanzee), with bovine and dog forming a sub-group and human and chimpanzee forming a second sub-group; (ii) Danio rerio (zebrafish), Ictalurus punctatus (channel catfish), Xenopus laevis (African clawed frog) and Callus gallus (chicken), with zebrafish, channel catfish and African clawed frog forming a sub-group and chicken forming a second sub-group; and (iii) Mus musculus (mouse), Rattus norvegicus (rat) and Oryctolagus cuniculus (rabbit), with mouse and rat forming a sub-group and rabbit forming a second sub-group. Thus, the skilled person can select moderately divergent species by reference to these groupings, eg,
a) the first AID is a wild-type AID (or functional mutant thereof) from a species in group (i) or a sub-group thereof and the second AID is a wild-type AID (or functional mutant thereof) from a species in group (ii) or (iii) or a sub-group thereof; or
b) the first AID is a wild-type AID (or functional mutant thereof) from a species in group (ii) or a sub-group thereof and the second AID is a wild-type AID (or functional mutant thereof) from a species in group (iii) or a sub-group thereof.

For example, the first AID is a wild-type human AID (or functional mutant thereof) and the second AID is a wild-type AID (or functional mutant thereof) from African clawed frog or chicken.

For example, the first AID is a wild-type mouse AID (or functional mutant thereof) and the second AID is a wild-type AID (or functional mutant thereof) from African clawed frog or chicken.

For example, the first AID is a wild-type rat AID (or functional mutant thereof) and the second AID is a wild-type AID (or functional mutant thereof) from African clawed frog or chicken.

For example, the first AID is a wild-type human AID (or functional mutant thereof) and the second AID is a wild-type AID (or functional mutant thereof) from rat or mouse.

Alternatively, the skilled person can select moderately divergent species by reference to sequence identity between AIDs or AID homologues from different species. Thus, in one embodiment, the first and second AIDs are wild-type AIDs from different species, wherein the amino acid sequences of the AIDs are at least 65% identical to each other, optionally at least 66, 67, 68, 69, 70, 71, 72, 73, 74, 75, 76, 77, 78, 79, 80, 81, 82, 83, 83, 84 or 85% identical to each other. Alternatively or additionally, optionally the amino acid sequences are no more than 95, 94, 93, 92, 91 or 90% identical to each other. For example, the amino acid sequences are at least 65% identical to each other, but no more than 95% identical to each other. This encompasses species that are moderately divergent such as human AID and a second AID selected from mouse, rat, rabbit, chicken and African clawed frog. In another example, the amino acid sequences are at least 68% identical to each other, but no more than 90% identical to each other. This encompasses a sub-set of species (e.g.,(e.g., human AID as the first AID and chicken or African clawed frog as the second AID) that are even more divergent and yet chosen to function in She vertebrate or vertebrate cell of the invention (e.g.,(e.g., a mouse or rat, or mouse or rat cell) to provide desirable diversity.

Thus, in one embodiment of the first configuration of the invention, the vertebrate is a mouse or a rat, or the vertebrate cell is a mouse cell or a rat cell and the first expressible gene encodes a human AID (e.g.,(e.g., SEQ ID NO: 12 in the sequence listing herein or a naturally-occurring polymorphic variant thereof; or SEQ ID NO: 1 or 2 disclosed in WO2010/113039) or a functional mutant thereof and the second expressible gene encodes a mouse, rat, rabbit, chicken or African clawed frog AID (SEQ ID NO: 16, 17, 18, 19 or 20 in the sequence listing herein, or a naturally-occurring polymorphic variant thereof) or functional mutant thereof. For example, the vertebrate is a mouse or a rat, or the vertebrate cell is a mouse cell or a rat cell and the first expressible gene encodes a human AID and the second expressible gene encodes a chicken AID. In another example, the vertebrate is a mouse or a rat, or the vertebrate cell is a mouse cell or a rat cell and the first expressible gene encodes a human AID and the second expressible gene encodes an African clawed frog AID. In another For example, the vertebrate is a mouse or a rat, or the vertebrate cell is a mouse cell or a rat cell and the first expressible gene encodes a human AID and the second expressible gene encodes mouse AID (e.g.,(e.g., AID endogenous to said mouse when said vertebrate is a mouse or vertebrate cell is a mouse cell). In another For example, the vertebrate is a mouse or a rat, or the vertebrate cell is a mouse cell or a rat cell and the first expressible gene encodes a human AID and the second expressible gene encodes rat AID (e.g.,(e.g., AID endogenous to said rat when said vertebrate is a rat or vertebrate cell is a rat cell).

In one embodiment of the first configuration, when the first and second expressible genes encode AID homologues,
a) the first AID homologue is a wild-type AID homologue (or functional mutant thereof) from a species in group (i) or a sub-group thereof and the second AID homologue is a wild-type AID homologue (or functional mutant thereof) from a species in group (ii) or (iii) or a sub-group thereof; or
b) the first AID homologue is a wild-type AID homologue (or functional mutant thereof) from a species in group (ii) or a sub-group thereof and the second AID homologue is a wild-type AID homologue (or functional mutant thereof) from a species in group (iii) or a sub-group thereof.

Suitable AID homologues include APOBEC1, APOBEC2, an APOBEC3, APOBEC3A, APOBEC3B, APOBEC3C, APOBEC3D, APOBEC3E, APOBEC3F, APOBEC3G, APOBEC3H and APOBEC4, provided that the first and second AID homologues are not identical.

For example, the first AID homologue is a wild-type human AID homologue (or functional mutant thereof) and the second AID homologue is a wild-type AID homologue (or functional mutant thereof) from African clawed frog or chicken.

For example, the first AID homologue is a wild-type mouse AID homologue (or functional mutant thereof) and the second AID homologue is a wild-type AID homologue (or functional mutant thereof) from African clawed frog or chicken.

For example, the first AID homologue is a wild-type rat AID homologue (or functional mutant thereof) and the second AID homologue is a wild-type AID homologue (or functional mutant thereof) from African clawed frog or chicken.

For example, the first AID homologue is a wild-type human AID homologue (or functional mutant thereof) and the second AID homologue is a wild-type AID homologue (or functional mutant thereof) from rat or mouse.

Alternatively, the skilled person can select moderately divergent species by reference to sequence identity between AID homologues from different species (for example, where the first and second homologues are the same APOBEC family member type, eg, both are an APOBEC1; or both are an APOBEC3, but are derived from different species). Moderate identity is an advantageous embodiment in which species are selected that are sufficiently divergent to provide for AID homologue diversity (and thus a resultant design for improved antibody diversity), and the considerations discussed above in relation to phylogenetic trees and sequence identity apply also to the choice of suitable AID homologues, as will be apparent to the skilled person in the light of the present disclosure. Thus, in one embodiment, the first and second AID homologues are wild-type AID homologues from different species (and optionally are the same APOBEC family member type), wherein the amino acid sequences of the AID homologues are at least 65% identical to each other, optionally at least 66, 67, 68, 69, 70, 71, 72, 73, 74, 75, 76, 77, 78, 79, 80, 81, 82, 83, 83, 84 or 85% identical to each other. Alternatively or additionally, optionally the amino acid sequences are no more than 95, 94, 93, 92, 91 or 90% identical to each other. For example, the amino acid sequences are at least 65% identical to each other, but no more than 95% identical to each other. This encompasses species that are moderately divergent such as human on the one hand and mouse, rat, rabbit, chicken or African clawed frog on the other hand. In another example, the amino acid sequences are at least 68% identical to each other, but no more than 90% identical to each other. This encompasses a sub-set of species (e.g.,(e.g., human for choice of the first AID homologue and chicken or African clawed frog as the second AID homologue) that are even more divergent and yet chosen to function in the vertebrate or vertebrate cell of the invention (e.g.,(e.g., a mouse or rat, or mouse or rat cell) to provide desirable diversity.

Thus, in one embodiment of the first configuration of the invention, the vertebrate is a mouse or a rat, or the vertebrate cell is a mouse cell or a rat cell and the first expressible gene encodes a human AID homologue or a functional mutant thereof and the second expressible gene encodes a mouse, rat, rabbit, chicken or African clawed frog AID homologue or functional mutant thereof. For example, the vertebrate is a mouse or a rat, or the vertebrate cell is a mouse cell or a rat cell and the first expressible gene encodes a human AID homologue (e.g.,(e.g., human APOBEC1) and the second expressible gene encodes a chicken AID homologue (e.g.,(e.g., chicken APOBEC1). In another For example, the vertebrate is a mouse or a rat, or the vertebrate cell is a mouse cell or a rat cell and the first expressible gene encodes a human AID homologue (e.g.,(e.g., human APOBEC1) and the second expressible gene encodes an African clawed frog AID homologue (e.g.,(e.g., African clawed frog APOBEC1). In another example, the vertebrate is a mouse or a rat, or the vertebrate cell is a mouse cell or a rat cell and the first expressible gene encodes a human AID homologue (e.g.,(e.g., human APOBEC1) and the second expressible gene encodes mouse AID homologue (e.g.,(e.g., a mouse APOBEC1, eg, AID homologue endogenous to said mouse when said vertebrate is a mouse or vertebrate cell is a mouse cell). In another For example, the vertebrate is a mouse or a rat, or the vertebrate cell is a mouse cell or a rat cell and the first expressible gene encodes a human AID homologue (e.g.,(e.g., human APOBEC1) and the second expressible gene encodes rat AID homologue (e.g.,(e.g., a rat APOBEC1, eg, AID homologue endogenous to said rat when said vertebrate is a rat or vertebrate cell is a rat cell).

In one embodiment, the first AID is a primate AID (e.g.,(e.g., SEQ ID NO: 12 or 13 in the sequence listing herein, or SEQ ID NO: 1, 2, 9 or 10 disclosed in WO2010/113039) or a functional mutant that is at least 95, 96, 97, 98 or 99% identical thereto or 100% identical thereto; and the second AID is murine AID (e.g.,(e.g., SEQ ID NO: 18 in the sequence listing herein, or SEQ ID NO: 4 disclosed in WO2010/113039) or a functional mutant that is at least 95, 96, 97, 98 or 99% identical thereto or 100% identical thereto. For example, the primate AID is selected from human, chimpanzee and macaque AID.

In one embodiment, the first AID is murine AID (e.g.,(e.g., SEQ ID NO: 18 in the sequence listing herein, or SEQ ID NO: 4 disclosed in WO2010/113039) or a functional mutant that is at least 95, 96, 97, 98 or 99% identical thereto or 100% identical thereto; and the second AID is human AID (e.g.,(e.g., SEQ ID NO: 12 in the sequence listing herein, or SEQ ID NO: 1 or 2 disclosed in WO2010/113039) or a functional mutant that is at least 95, 96, 97, 98 or 99% identical thereto or 100% identical thereto. In one embodiment, the first AID is murine AID (e.g.,(e.g., SEQ ID NO: 18 in the sequence listing herein, or SEQ ID NO: 4 disclosed in WO2010/113039); and the second AID is human AID (e.g., SEQ ID NO: 12 in the sequence listing herein, or SEQ ID NO: 1 or 2 disclosed in WO2010/113039),

In one embodiment, the first AID is a primate AID (e.g., SEQ ID NO: 12 or 13 in the sequence listing herein, or SEQ, ID NO: 1, 2, 9 or 10 disclosed in WO2010/113039) or a functional mutant that is at least 95, 96, 97, 98 or 99% identical thereto or 100% identical thereto; and the second AID is rat AID (e.g., SEQ ID NO: 17 in the sequence listing herein, or SEQ ID NO: 5 disclosed in WO2010/113039) or a functional mutant that is at least 95, 96, 97, 98 or 99% identical thereto or 100% identical thereto. For example, the primate AID is selected from human, chimpanzee and macaque AID.

In one embodiment, the first AID is rat AID (e.g., SEQ ID NO: 17 in the sequence listing herein, or SEQ ID NO: 5 disclosed in WO2010/113039) or a functional mutant that is at least 95, 96, 97, 98 or 99% identical thereto or 100% identical thereto; and the second AID is human AID (e.g., SEQ ID NO: 12 in the sequence listing herein, or SEQ ID NO: 1 or 2 disclosed in WO2010/113039) or a functional mutant that is at least 95, 96, 97, 98 or 99% identical thereto or 100% identical thereto. In one embodiment, the first AID is rat AID (e.g., SEQ ID NO: 17 in the sequence listing herein, or SEQ ID NO: 5 disclosed in WO2010/113039); and the second AID is human AID (e.g., SEQ ID NO: 12 in the sequence listing herein, or SEQ ID NO: 1 or 2 disclosed in WO2010/113039).

Optionally, for each AID mutant or AID homologue mutant in any configuration of the invention, the mutant retains a wild-type Hot Spot Recognition Loop. Reference is made to Kohli, R M et al, “A Portable Hot Spot Recognition loop Transfers Sequence Preference from APOBEC. Family Member to Activation-induced Cytidine Deaminase”, (2009) J. Biol. Chem. 284: 22898-22904; and to Holden, L G et al, “Crystal structure of the anti-viral APOBEC3G catalytic domain and functional implications”, (2008) Nature. 456:121-124, the disclosures of which are incorporated herein by reference, including the incorporation of Hot Spot Recognition Loop sequences as disclosed in these publications as though they are written explicitly herein as individual loop sequences (without flanking sequences) for use in the present invention and potential inclusion in claims herein. Thus, in one embodiment of the invention, the mutant retains a Hot Spot Recognition Loop (e.g., as disclosed in Kohli, R M et al) or an Active-Site Loop (e.g., as disclosed in Holden, L G et al).

In one embodiment, where the first and second AIDs or homologues are not identical, the constant region is provided by the constant region endogenous to the non-human vertebrate, eg, by inserting human V(D)J region sequences into operable linkage with an endogenous constant region of the non-human vertebrate genome or non-human vertebrate cell genome. In this embodiment, where there are human and non-human vertebrate regions in the transgene, advantageously the first AID or AID homologue is endogenous to the non-human vertebrate (or non-human vertebrate from which the cell of the invention is derived) or a functional mutant thereof; and the second AID is human AID (e.g., SEQ ID NO: 12 in the sequence listing herein, or SEQ ID NO: 1 or 2 disclosed in WO2010/113039) or a functional mutant that is at least 95, 96, 97, 98 or 99% identical thereto or 100% identical thereto. This provides for an enhanced spectrum of AID or homologue activity in a way that matches the origins of the enzymes to the substrate sequences on which they act in the non-human vertebrate or cell (e.g., mouse or rat; or mouse cell or rat cell). The inventors believe that such an enhanced activity spectrum provides for greater sequence diversity generated by SHM and/or CSR. Greater diversity is useful for providing diversity of antibodies which can be selected against a predetermined target antigen. This may be desirable where high affinity antibodies are sought and/or antibodies to epitopes that are not readily accessed by existing in vivo and in vitro antibody selection systems. Examples of possible embodiments are as follows.

In a first embodiment, where the first and second AIDs or homologues are not identical, the constant region is provided by the constant region endogenous to a mouse, eg, by inserting human V(D)J region sequences into operable linkage with the endogenous constant region of a mouse genome or mouse cell genome. In this embodiment, where there are human and mouse regions, advantageously the first AID or AID homologue is endogenous to the mouse (or mouse from which the cell is derived) or a functional mutant thereof; and the second AID is human AID (e.g., SEQ ID NO: 12 in the sequence listing herein, or SEQ ID NO: 1 or 2 disclosed in WO2010/113039) or a functional mutant that is at least 95, 96, 97, 98 or 99% identical thereto or 100% identical thereto. In one example, the vertebrate is a mouse and the first AID or homologue is a mouse AID or AID homologue (e.g., SEQ ID NO: 18 in the sequence listing herein; or SEQ ID NO: 4 disclosed in WO2010/113039; or an AID or AID homologue endogenous to said mouse) and the second AID or homologue is a human AID or AID homologue (e.g., SEQ ID NO: 12 in the sequence listing herein, or SEQ ID NO: 1 or 2 disclosed in WO2010/113039). Instead of reference to “human AID or AID homologue” in this paragraph, in an alternative a primate AID or AID homologue is used, eg, where the primate is chimpanzee or macaque.

In a second embodiment, where the first and second AIDs or homologues are not identical, the constant region is provided by the constant region endogenous to a rat, eg, by inserting human V(D)J region sequences into operable linkage with the endogenous constant region of a rat genome or rat cell genome. In this embodiment, where there are human and rat regions, advantageously the first AID or AID homologue is endogenous to the rat (or rat from which the cell is derived) or a functional mutant thereof; and the second AID is human AID (e.g., SEQ ID NO: 12 in the sequence listing herein, or SEQ ID NO: 1 or 2 disclosed in WO2010/113039) or a functional mutant that is at least 95, 96, 97, 98 or 99% identical thereto or 100% identical thereto. In one example, the vertebrate is a rat and the first AID or homologue is a rat AID or AID homologue (e.g., SEQ, ID NO: 17 in the sequence listing herein; or SEQ ID NO: 5 disclosed in WO2010/113039; or an AID or AID homologue endogenous to said rat) and the second AID or homologue is a human AID or AID homologue (e.g., SEQ ID NO: 12 in the sequence listing herein, or SEQ ID NO: 1 or 2 disclosed in WO2010/113039). Instead of reference to “human AID or AID homologue” in this paragraph, in an alternative a primate AID or AID homologue is used, eg, where the primate is chimpanzee or macaque.

In an aspect of the first configuration of the invention, there is provided a transgenic mouse or mouse cell, comprising

(a) a transgene, wherein the transgene comprises substantially the full human repertoire of IgH V, D and J regions, wherein said regions are upstream of a constant region, wherein the constant region is a mouse constant region or derived from a mouse constant region, optionally comprising a mouse Sμ switch and/or optionally a mouse Cμ region;
(b) a first expressible gene encoding a first activation-induced deaminase (AID) or an AID homologue; and
(c) a second expressible gene encoding a second AID or an AID homologue, wherein the first and second AIDs or AID homologues are not identical.

In an aspect of the first configuration of the invention, there is provided a transgenic rat or rat cell, comprising

(a) a transgene, wherein the transgene comprises substantially the full human repertoire of IgH V, D and J regions, wherein said regions are upstream of a constant region, wherein the constant region is a rat constant region or derived from a rat constant region, optionally comprising a rat Sμ switch and/or optionally a rat Cμ region;
(b) a first expressible gene encoding a first activation-induced deaminase (AID) or an AID homologue; and
(c) a second expressible gene encoding a second AID or an AID homologue, wherein She first and second AIDs or AID homologues are not identical.

A second configuration of the invention provides a transgenic non-human vertebrate or vertebrate cell whose genome comprises

(a) a transgene, wherein the transgene comprises at least one human V region, at least one human J region, and optionally at least one human D region, wherein said regions are upstream of a constant region;
(b) a first expressible gene encoding a first activation-induced deaminase (AID) or an AID homologue; and
(c) a second expressible gene encoding a second AID or an AID homologue,
wherein each AID or AID homologue is either (i) a human AID or AID homologue, or a functional mutant thereof; or (ii) a mouse AID or AID homologue, or a functional mutant thereof when the vertebrate is a mouse or cell is a mouse cell, and the first and second AIDs or homologues are not identical; or (iii) a rat AID or AID homologue, or a functional mutant thereof when the vertebrate is a rat or cell is a rat cell, and the first and second AIDs or homologues are not identical; and
optionally wherein the transgene comprises instead a rearranged VDJ or VJ nucleotide sequence.

Optionally in this second configuration of the invention where (i) applies (human AID or homologue), the first and second AIDs or homologues are not identical.

An aspect of the second configuration provides a transgenic mouse or mouse cell comprising

(a) a transgene, wherein the transgene comprises substantially the full human repertoire of IgH V, D and J regions, wherein said regions are upstream of a constant region, wherein the constant region is a mouse constant region or derived from a mouse constant region, optionally comprising a mouse Sμ switch and/or optionally a mouse Cμ region;
(b) a first expressible gene encoding a first activation-induced deaminase (AID) or an AID homologue; and
(c) a second expressible gene encoding a second AID or an AID homologue,
wherein each AID or AID homologue is a human AID or AID homologue, or a functional mutant thereof.

An aspect of the second configuration provides a transgenic rat or rat cell comprising

(a) a transgene, wherein the transgene comprises substantially the full human repertoire of IgH V, D and J regions, wherein said regions are upstream of a constant region, wherein the constant region is a rat constant region or derived from a rat constant region, optionally comprising a rat Sμ switch and/or optionally a rat Cμ region;
(b) a first expressible gene encoding a first activation-induced deaminase (AID) or an AID homologue; and
(c) a second expressible gene encoding a second AID or an AID homologue,
wherein each AID or AID homologue is a human AID or AID homologue, or a functional mutant thereof.

Optionally in the first or second configuration of the invention, either (i) the vertebrate is a mouse, the constant region is a mouse constant region or derived from a mouse constant region, and the first expressible AID or AID homologue gene is a mouse AID or AID homologue gene; optionally wherein the first AID or AID homologue gene and constant region are derived from the same mouse strain; or (ii) the vertebrate is a rat, the constant region is a rat constant region or derived from a rat constant region, and the first expressible AID or AID homologue gene is a rat AID or AID homologue gene; optionally wherein the first AID or AID homologue gene and constant region are derived from the same mouse rat strain.

Optionally in the first configuration of the invention, the first AID or AID homologue gene is the wild-type AID gene. Additionally or alternatively, optionally the second AID or AID homologue gene comprises the nucleotide sequence of a human AID, human APOBEC1, human APOBEC3C, human APOBEC3F, human APOBEC3G, or a functional mutant that is at least 95, 96, 97, 98 or 99% identical thereto or 100% identical thereto identical thereto.

Optionally in the second configuration of the invention, the first and/or second AID or AID homologue genes are the wild-type AID human gene. Additionally or alternatively, optionally the first and/or second AID or AID homologue gene comprises the nucleotide sequence of human AID, human APOBEC1, human APOBEC3C, human APOBEC3F, human APOBEC3G, or a functional mutant that is at least 95, 96, 97, 98 or 99% identical thereto or 100% identical thereto identical thereto.

In one embodiment in any configuration of the invention, the vertebrate is a mouse, rat, rabbit Camelid (e.g., a llama, alpaca or camel), shark, or the vertebrate cell is a mouse, rat, rabbit Camelid (e.g., a llama, alpaca or camel), shark cell.

In one aspect the only human DNA inserted into the non-human vertebrate cell or animal are V, D or J coding regions, and these are placed under control of the host regulatory sequences or other (non-human, non-host) sequences. In one aspect reference to human coding regions includes both human introns and exons, or in another aspect simply exons and no introns, which may be in the form of cDNA.

Alternatively it is possible to use recombineering, or other recombinant DNA technologies, to insert a non human-vertebrate (e.g. mouse) promoter or other control region, such as a promoter for a V region, into a BAC containing a human Ig region. The recombineering step then places a portion of human DNA under control of the mouse promoter or other control region.

The invention also relates to a cell line which is grown from or otherwise derived from cells as described herein, including an immortalised cell line. The cell line may comprise inserted human V, D or J genes as described herein, either in germline configuration or after rearrangement following in vivo maturation. The cell may be immortalised by fusion to a tumour cell to provide an antibody producing cell and cell line, or be made by direct cellular immortalisation.

In one aspect the non-human vertebrate of any configuration of the invention is able to generate a diversity of at least 1×10⁶ different functional chimaeric immunoglobulin sequence combinations.

Optionally in any configuration of the invention the constant region is endogenous to the vertebrate and optionally comprises an endogenous switch. In one embodiment, the constant region comprises a Cgamma (Cγ) region and/or a Smu (Sμ) switch. Switch sequences are known in the art, for example, see Nikaido et al, Nature 292: 845-848 (1981) and also co-pending application PCT/GB2010/051122, U.S. Pat. Nos. 7,501,552, 6,673,986, 6,130,364, WO2009/076464 and U.S. Pat. No. 6,586,251, eg, SEQ ID NOs: 9-24 disclosed in US750.1552. Optionally She constant region comprises an endogenous S gamma switch and/or an endogenous Smu switch. One or more endogenous switch regions can be provided, in one embodiment, by constructing a transgenic immunoglobulin locus in the vertebrate or cell genome in which at least one human V region, at least one human J region, and optionally at least one human D region, or a rearranged VDJ or VJ region, are inserted into the genome in operable linkage with a constant region that is endogenous to the vertebrate or cell. For example, the human V(D)J regions or rearranged VDJ or VJ can be inserted in a as orientation onto the same chromosome as the endogenous constant region. A trans orientation is also possible, in which the human V(D)J regions or rearranged VDJ or VJ are inserted into one chromosome of a pair (e.g., the chromosome 6 pair in a mouse or the chromosome 4 in a rat) and the endogenous constant region is on the other chromosome of the pair, such that trans-switching takes place in which the human V(D)J regions or rearranged VDJ or VJ are spliced inoperable linkage to the endogenous constant region. In this way, the vertebrate can express antibodies having a chain that comprises a variable region encoded all or in part by human V(D)J or a rearranged VDJ or VJ, together with a constant region (e.g., a Cgamma or Cmu) that is endogenous to the vertebrate.

Human variable regions are suitably inserted upstream of non-human vertebrate constant region, the latter comprising all of the DNA required to encode the full constant region or a sufficient portion of the constant region to allow the formation of an effective chimaeric antibody capable of specifically recognising an antigen.

In one aspect the chimaeric antibodies or antibody chains have a part of a host constant region sufficient to provide one or more effector functions seen in antibodies occurring naturally in a host vertebrate, for example that they are able interact with Fc receptors, and/or bind to complement.

Reference to a chimaeric antibody or antibody chain having a host non-vertebrate constant region herein therefore is not limited to the complete constant region but also includes chimaeric antibodies or chains which have all of the host constant region, or a part thereof sufficient to provide one or more effector functions. This also applies to non-vertebrate mammals and cells and methods of the invention in which human variable region DNA may be inserted into the host genome such that it forms a chimaeric antibody chain with all or part of a host constant region, in one aspect the whole of a host constant region is operably linked to human variable region DNA.

The host non-human vertebrate constant region herein is optionally the endogenous host wild-type constant region located at the wild type locus, as appropriate for the heavy or light chain. For example, the human heavy chain DNA is suitably inserted on mouse chromosome 12, suitably adjacent the mouse heavy chain constant region, where the vertebrate is a mouse.

In one optional aspect where the vertebrate is a mouse, the insertion of the human DNA, such as the human VDJ region is targeted to the region between the J4 exon and the Cμ locus in the mouse genome IgH locus, and in one aspect is inserted between coordinates 114,667,090 and 114,665,190, suitably at coordinate 114,667,091. In one aspect the insertion of the human DNA, such as the human light chain kappa VJ is targeted into mouse chromosome 6 between coordinates 70,673,899 and 70,675,515, suitably at position 70,674,734, or an equivalent position in the lambda mouse locus on chromosome 16. in one aspect the host non-human vertebrate constant region for forming the chimaeric antibody may be at a different (non endogenous) chromosomal locus. In this case the inserted human DMA, such as the human variable VDJ or VJ region(s) may then be inserted into the non-human genome at a site which is distinct from that of the naturally occurring heavy or light constant region. The native constant region may be inserted into the genome, or duplicated within the genome, at a different chromosomal locus to the native position, such that it is in a functional arrangement with the human variable region such that chimaeric antibodies of the invention can still be produced,

In one aspect the human DNA is inserted at the endogenous host wild-type constant region located at the wild type locus between the host constant region and the host VDJ region.

Reference to location of the variable region upstream of the non-human vertebrate constant region means that there is a suitable relative location of the two antibody portions, variable and constant, to allow the variable and constant regions to form a chimaeric antibody or antibody chain in vivo in the mammal. Thus, the inserted human DNA and host constant region are in functional arrangement with one another for antibody or antibody chain production.

In one aspect the inserted human DNA is capable of being expressed with different host constant regions through isotype switching. In one aspect isotype switching does not require or involve trans switching. Insertion of the human variable region DNA on the same chromosome as the relevant host constant region means that there is no need for trans-switching to produce isotype switching.

In the present invention, optionally host non-human vertebrate constant regions are maintained and it is preferred that at least one non-human vertebrate enhancer or other control sequence, such as a switch region, is maintained in functional arrangement with the non-human vertebrate constant region, such that the effect of the enhancer or other control sequence, as seen in the host vertebrate, is exerted in whole or in part in the transgenic animal. This approach is designed to allow the full diversity of the human locus to be sampled, to allow the same high expression levels that would be achieved by non-human vertebrate control sequences such as enhancers, and is such that signalling in the B-cell, for example isotype switching using switch recombination sites, would still use non-human vertebrate sequences,

A mammal having such a genome would produce chimaeric antibodies with human variable and non-human vertebrate constant regions, but these are readily humanized, for example in a cloning step. Moreover the in vivo efficacy of these chimaeric antibodies could be assessed in these same animals.

In one aspect the inserted human IgH VDJ region comprises, in germline configuration, all of the V, D and J regions and intervening sequences from a human.

In one aspect 800-1000 kb of the human IgH VDJ region is inserted into the non-human vertebrate IgH locus, and in one aspect a 940, 950 or 960 kb fragment is inserted. Suitably this includes bases 105,400,051 to 106,368,585 from human chromosome 14 (all coordinates refer to NCBI36 for the human genome, ENSEMBL Release 54 and NCBIM37 for the mouse genome, relating to mouse strain C57BL/6J).

In one aspect the inserted IgH human fragment consists of bases 105,400,051 to 106,368,585 from chromosome 14. In one aspect the inserted human heavy chain DNA, such as DNA consisting of bases 105,400,051 to 106,368,585 from chromosome 14, is inserted into mouse chromosome 12 between the end of the mouse J4 region and the Eμ region, suitably between coordinates 114,667,091 and 114,665,190, suitably at coordinate 114,667,091.

In one aspect the inserted human kappa VJ region comprises, in germline configuration, all of the V and J regions and intervening sequences from a human.

Suitably this includes bases 88,940,356 to 89,857,000 from human chromosome 2, suitably approximately 917 kb. In a further aspect the light chain VJ insert may comprise only the proximal clusters of V segments and J segments. Such an insert would be of approximately 473 kb.

In one aspect the human light chain kappa DMA, such as the human IgK fragment of bases 88,940,356 to 89,857,000 from human chromosome 2, is suitably inserted into mouse chromosome 6 between coordinates 70,673,899 and 70,675,515, suitably at position 70,674,734.

In one aspect the human lambda VJ region comprises, in germline configuration, all of the V and J regions and intervening sequences from a human. Suitably this includes analogous bases to those selected for the kappa fragment, from human chromosome 2.

All specific human fragments described above may vary in length, and may for example be longer or shorter than defined as above, such as 500 bases, 1 KB, 2K, 3K, 4K, 5 KB, 10 KB, 20 KB, 30 KB, 40 KB or 50 KB or more, which suitably comprise all or part of the human V(D)J region, whilst preferably retaining the requirement for the final insert to comprise human genetic material encoding the complete heavy chain region and light chain region, as appropriate, as described above.

In one aspect the 3′ end of the last inserted human sequence, generally the last human J sequence, is inserted less than 2 kb, preferably less than 1 KB from the human/non-human vertebrate (e.g., human/mouse or human/rat) join region.

Optionally, the genome is homozygous at one, or both, or all three immunoglobulin loci (IgH, Ig λ and Igκ).

In another aspect the genome may be heterozygous at one or more of the loci, such as heterozygous for DNA encoding a chimaeric antibody chain and native (host cell) antibody chain, in one aspect the genome may be heterozygous for DNA capable of encoding 2 different antibody chains encoded by transgenes of the invention, for example, comprising 2 different chimaeric heavy chains or 2 different chimaeric light chains.

In one aspect the invention relates to a non-human vertebrate or cell, and methods for producing said vertebrate or cell, as described herein, wherein the inserted human DNA, such as the human IgH VDJ region and/or light chain V, J regions are found on only one allele and not both alleles in the mammal or cell. In this aspect a mammal or cell has the potential to express both an endogenous host antibody heavy or light chain and a chimaeric heavy or light chain.

In one embodiment in any configuration of the invention, She genome has been modified to prevent or reduce the expression of fully-endogenous antibody. Examples of suitable techniques for doing this can be found in PCT/GB2010/051122, U.S. Pat. Nos. 7,501,552, 6,673,986, 6,130,364, WO2009/076464, EP1399559 and U.S. Pat. No. 6,586,251, the disclosures of which are incorporated herein by reference. In one embodiment, the non-human vertebrate VDJ region of the endogenous heavy chain immunoglobulin locus, and optionally VJ region of the endogenous light chain immunoglobulin loci (lambda and/or kappa loci), have been inactivated. For example, all or part of the non-human vertebrate VDJ region is inactivated by inversion in the endogenous heavy chain immunoglobulin locus of the mammal, optionally with the inverted region being moved upstream or downstream of the endogenous Ig locus. For example, all or part of the non-human vertebrate VJ region is inactivated by inversion in the endogenous kappa chain immunoglobulin locus of the mammal, optionally with the inverted region being moved upstream or downstream of the endogenous Ig locus. For example, all or part of the non-human vertebrate VJ region is inactivated by inversion in the endogenous lambda chain immunoglobulin locus of the mammal, optionally with the inverted region being moved upstream or downstream of the endogenous Ig locus. In one embodiment the endogenous heavy chain locus is inactivated in this way as is one or both of She endogenous kappa and lambda loci.

Additionally or alternatively, the vertebrate has been generated in a genetic background which prevents the production of mature host B and T lymphocytes, optionally a RAG-1-deficient and/or RAG-2 deficient background. See U.S. Pat. No. 5,859,301 for techniques of generating RAG-1 deficient animals.

In one embodiment in any configuration of the invention, the human V, J and optional D regions are provided by all or part of the human IgH locus; optionally wherein said all or part of the IgH locus includes substantially the full human repertoire of IgH V, D and J regions and intervening sequences. A suitable part of the human IgH locus is disclosed in PCT/GB2010/051122. In one embodiment, the human IgH part includes (or optionally consists of) bases 105,400,051 to 106,368,585 from human chromosome 14 (coordinates from NCBI36). Additionally or alternatively, optionally wherein the vertebrate is a mouse or the cell is a mouse cell, the human V, J and optional D regions are inserted into mouse chromosome 12 at a position corresponding to a position between coordinates 114,667,091 and 114,665,190, optionally at coordinate 114,667,091 (coordinates from NCBIM137, relating to mouse strain C57BL/6J).

In one embodiment of any configuration of the vertebrate or vertebrate cell of the invention when the vertebrate is a mouse, (i) the constant region comprises a mouse Sμ switch and optionally a mouse Cμ region. For example the constant region is provided by the constant region endogenous to the mouse, eg, by inserting human V(D)J region sequences into operable linkage with the endogenous constant region of a mouse genome or mouse cell genome.

In one embodiment of any configuration of the vertebrate or vertebrate cell of the invention when the vertebrate is a rat, (i) the constant region comprises a rat Sμ switch and optionally a rat Cμ region. For example the constant region is provided by the constant region endogenous to the rat, eg, by inserting human V(D)J region sequences into operable linkage with the endogenous constant region of a rat genome or rat cell genome.

In one embodiment of any configuration of the vertebrate or vertebrate cell of the invention the transgene comprises all or part of the human Igλ locus including at least one human Jλ region and at least one human Cλ region, optionally C_{λ}6 and/or C_{λ}7. Optionally, the transgene comprises a plurality of human Jλ regions, optionally two or more of J_{λ}1, J_{λ}2, J_{λ}6 and J_{λ}7, optionally all of J_{λ}1, J_{λ}2, J_{λ}6 and J_{λ}7. The human lambda immunoglobulin locus comprises a unique gene architecture composed of serial J-C clusters. In order to take advantage of this feature, the invention in optional aspects employs one or more such human J-C clusters inoperable linkage with the constant region in the transgene, eg, where the constant region is endogenous to the non-human vertebrate or non-human vertebrate cell. Thus, optionally the transgene comprises at least one human J_{λ}-C_{λ} cluster, optionally at least J_{λ}7-C_{λ}7. The construction of such transgenes is facilitated by being able to use all or part of the human lambda locus such that the transgene comprises one or more J-C clusters in germline configuration, advantageously also including intervening sequences between clusters and/or between adjacent J and C regions in the human locus. This preserves any regulatory elements within the intervening sequences which may be involved in VJ and/or JC recombination and which may be recognised by AID or AID homologues.

Where endogenous regulatory elements are involved in CSR in the non-human vertebrate, these can be preserved by including in the transgene a constant region that is endogenous to the non-human vertebrate. In the first configuration of the invention, one can match this by using an AID or AID homologue that is endogenous to the vertebrate or a functional mutant thereof. Such design elements of the present invention are advantageous for maximising the enzymatic spectrum for SHM and/or CSR and thus for maximising the potential for antibody diversity.

Optionally, the transgene comprises a human Eλ enhancer.

In one embodiment of any configuration of the invention the constant region is a human constant region or derived from a human constant region,

In one embodiment of any configuration of the invention the constant region is endogenous to the non-human vertebrate or derived from such a constant region. For example, the vertebrate is a mouse or the cell is a mouse cell and the constant region is endogenous to the mouse. For example, the vertebrate is a rat or the cell is a rat cell and the constant region is endogenous to the rat.

In one embodiment of any configuration of the invention the transgene comprises at least one human IgH V region, at least one human D region and at least one human J region.

In one embodiment of any configuration of the invention the transgene comprises a plurality human IgH V regions, a plurality of human D regions and a plurality of human J regions, optionally substantially the full human repertoire of IgH V, D and J regions.

In one embodiment of any configuration of the invention, the vertebrate or cell comprises a further transgene, the further transgene comprising at least one human IgH V region, at least one human D region and at least one human J region, optionally substantially the full human repertoire of IgH V, D and J regions.

In one embodiment of any configuration of the invention,

(i) the transgene comprises at least one human IgH V region, at least one human J region, and optionally at least one human D region; and
(ii) the vertebrate or cell comprises a further transgene, the further transgene comprising at least one human Igκ V region and at least one human J region.

In one embodiment of any configuration of the invention,

(i) the transgene comprises at least one human IgH V region, at least one human J region, and optionally at least one human D region; and
(ii) the vertebrate or cell comprises a further transgene, the further transgene comprising at least one human Igλ V region and at least one human J region.

In one embodiment of any configuration of the invention,

(i) the transgene comprises substantially the full human repertoire of IgH V, D and J regions; and
(ii) the vertebrate or cell comprises substantially the full human repertoire of Igκ V and J regions and/or substantially the full human repertoire of Ig λ V and J regions.

In one embodiment of the second configuration of the invention, the first expressible gene encodes a human AID (e.g., SEQ ID NO: 12 in the sequence listing herein; or SEQ ID NO: 1 or 2 disclosed in WO2010/113039) and the second expressible gene encodes a functional mutant of human AID comprising an amino acid sequence that is at least 95, 96, 97, 98 or 99% identical thereto; or wherein the first expressible gene encodes an AID homologue selected from human APOBEC1, human APOBEC3C, human APOBEC3F and human APOBEC3G and the second expressible gene encodes a functional AID homologue mutant comprising an amino acid sequence that is at least 95, 96, 97, 98 or 99% identical thereto; or wherein the first expressible gene encodes a human AID (e.g., SEQ ID NO: 12 in the sequence listing herein; or SEQ ID NO: 1 or 2 disclosed in WO2010/113039) or a functional mutant comprising an amino acid sequence that is at least 95, 96, 97, 98 or 99% identical thereto, and the second expressible gene encodes an AID homologue selected from human APOBEC1, human APOBEC3C, human APOBEC3F and human APOBEC3G or a functional mutant comprising an amino acid sequence that is at least 95, 96, 97, 98 or 99% identical thereto. Optionally, each AID is a functional mutant comprising an amino acid sequence that is at least 95, 96, 97, 98 or 99% identical to SEQ ID NO: 12 in the sequence listing herein or SEQ ID NO: 1 or 2 disclosed in WO2010/113039; or each AID homologue is a functional mutant comprising an amino acid sequence that is at least 95, 96, 97, 98 or 99% identical to a human APOBEC1, human APOBEC3C, human APOBEC3F or human APOBEC3G. Optionally, the first and second expressible genes encode human AIDs and each AID is a wild-type human AID (SEQ ID NO: 12). Optionally, the first and second expressible genes encode human APOBEC1 and each APOBEC1 is a wild-type human APOBEC1. Optionally, the first and second expressible genes encode human APOBEC2 and each APOBEC2 is a wild-type human APOBEC2. Optionally, the first and second expressible genes encode human APOBEC3 and each APOBEC3 is a wild-type human APOBEC3. Optionally, the first and second expressible genes encode human APOBEC3A h APOBEC3A is a wild-type human APOBEC3A. Optionally, the first and second expressible genes encode human APOBEC3B and each APOBEC3B is a wild-type human APOBEC3B. Optionally, the first and second expressible genes encode human APOBEC3C and each APOBEC3C is a wild-type human APOBEC3C. Optionally, the first and second expressible genes encode human APOBEC3D and each APOBEC3D is a wild-type human APOBEC3D. Optionally, the first and second expressible genes encode human APOBEC3E and each APOBEC3E is a wild-type human APOBEC3E. Optionally, the first and second expressible genes encode human APOBEC3F and each APOBEC3F is a wild-type human APOBEC3F. Optionally, the first and second expressible genes encode human APOBEC3G and each APOBEC3G is a wild-type human APOBEC3G. Optionally, the first and second expressible genes encode human APOBEC3H and each APOBEC3H is a wild-type human APOBEC3H. Optionally, the first and second expressible genes encode human APOBEC4 and each APOBEC4 is a wild-type human APOBEC4.

In an aspect of any configuration of the invention, the expression of at least one of the AIDs or AID homologues is inducible. For example, each AID or AID homologue gene is inducible. This may be beneficial to harness the desirable SHM and CSR effects of the enzymes while reducing or avoiding over-activity that may lead to detrimental effects such as chromosomal translocation.

In an aspect of any configuration of the invention, at least one or each AID, AID homologue or mutant is present in the genome under operable control of wild-type AID gene control elements, eg, where the non-human vertebrate is a mouse (or for a mouse cell), She control elements are AID gene control elements endogenous to the mouse; or where the non-human vertebrate is a rat (or for a rat cell), the control elements are AID gene control elements endogenous to the rat. In this way, for example where each AID, AID homologue or mutant gene is under the control of an endogenous AID control element, one can harness the endogenous control mechanisms of the non-human vertebrate thereby regulating the expression and/or activity of the first and second AID, AID homologue or mutant. This may be beneficial to harness the desirable SHM and CSR effects of the enzymes while reducing or avoiding over-activity that may lead to undesirable effects such as chromosomal translocation.

Reference is made to R Maul & P Gearhart, Advances in immunology, 2010, volume 105, Chapter 6 (pp 159-191): AID and Somatic Hypermutation, which reviews AID and discloses codon preference. In this respect, reference is also made to WO2008/103475. One embodiment of any configuration of the invention uses codon preference to provide for improved AID, homologue or mutant activity. To this end, optionally in the vertebrate or cell of the invention at least one V, D and/or J region sequence in the (or each) transgene has been codon-optimised for AID or an AID homologue or mutant thereof, optionally wherein the V, D and/or J sequence has been changed to include a sequence motif selected from the group consisting of DGYW, WRC, WRCY, WRCH, RGYW, AGY,TAC, WGCW, wherein W=A or T, Y=C or T, D=A, G or T, H=A or C or T, and R=A or G.

An aspect provides a B-cell, hybridoma or a stem cell, optionally an embryonic stem cell or haematopoietic stem cell, according to any configuration of the invention. In one embodiment, the cell is a JM8 or AB2.1 embryonic stem cell (see discussion of suitable cells, and in particular JM8 and AB2.1 cells, in PCT/GB2010/051122, which disclosure is incorporated herein by reference).

In one aspect the ES cell is derived from the mouse C57BL/6N, C57BL/6J, 129S5 or 129Sv strain.

In one aspect the non-human vertebrate is a rodent, suitably a mouse, and cells of the invention, are rodent cells or ES cells, suitably mouse ES cells.

The ES cells of the present invention can be used to generate animals using techniques well known in the art, which comprise injection of the ES cell into a blastocyst followed by implantation of chimaeric blastocysts into females to produce offspring which can be bred and selected for homozygous recombinants having the required insertion. In one aspect the invention relates to a transgenic animal comprised of ES cell-derived tissue and host embryo derived tissue. In one aspect the invention relates to genetically-altered subsequent generation animals, which include animals having a homozygous recombinants for the VDJ and/or VJ regions.

An aspect provides a method of isolating an antibody or nucleotide sequence encoding said antibody, the method comprising

(a) immunising (see e.g. Harlow, E, & Lane, D, 1998, 5^{th} edition, Antibodies: A Laboratory Manual, Cold Spring Harbor Lab, Press, Plainview, N.Y.; and Pasqualini and Arap, Proceedings of the National Academy of Sciences (2004) 101:257-259) a vertebrate according to any configuration or aspect of the invention with an antigen such that the vertebrate produces antibodies; and
(b) Isolating from the vertebrate an antibody that specifically binds to said antigen and/or a nucleotide sequence encoding at least the heavy and/or the light chain variable regions of said antibody;
optionally wherein the variable regions of said antibody are subsequently joined to a human constant region. Such joining can be effected by techniques readily available in the art, such as using conventional recombinant DMA and RNA technology as will be apparent to the skilled person. See e.g. Sambrook, J and Russell, D, (2001, 3'd edition) Molecular Cloning: A Laboratory Manual (Cold Spring Harbor Lab. Press, Plainview, N.Y.).

Suitably an immunogenic amount of the antigen is delivered. The invention also relates to a method for detecting a target antigen comprising detecting an antibody produced as above with a secondary-detection agent which recognises a portion of that antibody.

Isolation of the antibody in step (b) can be carried out using conventional antibody selection techniques, eg, panning for antibodies against antigen that has been immobilised on a solid support, optionally with iterative rounds at increasing stringency, as will be readily apparent to the skilled person.

As a further optional step, after step (b) the amino acid sequence of the heavy and/or the light chain variable regions of the antibody are mutated to improve affinity for binding to said antigen. Mutation can be generated by conventional techniques as will be readily apparent to the skilled person, eg, by error-prone PCR, Affinity can be determined by conventional techniques as will be readily apparent to the skilled person, eg, by surface plasmon resonance, eg, using Biacore™.

Additionally or alternatively, as a further optional step, after step (b) the amino acid sequence of the heavy and/or the light chain variable regions of the antibody are mutated to improve one or more biophysical characteristics of the antibody, eg, one or more of melting temperature, solution state (monomer or dimer), stability and expression (e.g., in CHO or E coli).

An aspect provides an antibody produced by the method of the invention, optionally for use in medicine, eg, for treating and/or preventing a medical condition or disease in a patient, eg, a human.

An aspect provides a nucleotide sequence encoding the antibody of the invention, optionally wherein the nucleotide sequence is part of a vector. Suitable vectors will be readily apparent to the skilled person, eg, a conventional antibody expression vector comprising the nucleotide sequence together in operable linkage with one or more expression control elements.

An aspect provides a pharmaceutical composition comprising the antibody of the invention and a diluent, excipient or carrier.

An aspect provides the use of the antibody of the invention in the manufacture of a medicament for the treatment and/or prophylaxis of a disease or condition in a patient, eg a human.

In a further aspect the invention relates to humanised antibodies and antibody chains produced according to the present invention, both in chimaeric and fully humanised form, and use of said antibodies in medicine. The invention also relates to a pharmaceutical composition comprising such an antibody and a pharmaceutically acceptable carrier or other excipient.

Antibody chains containing human sequences, such as chimaeric human-non human antibody chains, are considered humanised herein by virtue of the presence of the human protein coding regions region. Fully humanised antibodies may be produced starting from DNA encoding a chimaeric antibody chain of the invention using standard techniques.

Methods for the generation of both monoclonal and polyclonal antibodies are well known in the art, and the present invention relates to both polyclonal and monoclonal antibodies of chimaeric or fully humanised antibodies produced in response to antigen challenge in non human-vertebrates of the present invention.

In a yet further aspect, chimaeric antibodies or antibody chains generated in the present invention may be manipulated, suitably at the DNA level, to generate molecules with antibody-like properties or structure, such as a human variable region from a heavy or light chain absent a constant region, for example a domain antibody; or a human variable region with any constant region from either heavy or light chain from the same or different species; or a human variable region with a non-naturally occurring constant region; or human variable region together with any other fusion partner. The invention relates to all such chimaeric antibody derivatives derived from chimaeric antibodies identified according to the present invention.

In a further aspect, the invention relates to use of animals of the present invention in the analysis of the likely effects of drugs and vaccines in the context of a quasi-human antibody repertoire.

The invention also relates to a method for identification or validation of a drug or vaccine, the method comprising delivering the vaccine or drug to a mammal of the invention and monitoring one or more of: the immune response, the safety profile; the effect on disease.

The invention also relates to a kit comprising an antibody or antibody derivative as disclosed herein and either instructions for use of such antibody or a suitable laboratory reagent, such as a buffer, antibody detection reagent.

AID and AID Homologues

The nucleotide and amino acid sequences of human, mouse, rat and other AIDs are given below (SEQ ID NOs: 1-22. The term “AID” includes wild-type AID proteins (including naturally-occurring polymorphic variants) as well as functional AID mutants. In one embodiment, a functional AID mutant has an amino acid sequence that is at least 90% (optionally at least 95%, 96%, 97%, 98% or 99%) identical to the amino acid sequence of a wild-type AID (e.g., a wild-type human, rat, mouse or other vertebrate or mammal AID sequence disclosed herein).

The entire disclosure of WO2010/113039 is incorporated herein by reference. Reference is made in particular to FIG. 8 of WO2010/113039, the disclosure of which is incorporated herein in its entirety, including all information disclosed in each listed Genbank entry, including incorporation of named publications and each nucleotide and amino acid sequence disclosed in the Genbank entry as though such sequences are explicitly written herein for use in the present invention and as basis for potential incorporation into claims below.

Reference is also made to the wild-type AID sequences (SEQ ID NOs: 1 to 14) disclosed in WO2010/113039, each AID nucleotide and amino acid sequence disclosed in WO2010/113039 being incorporated herein by reference as though such sequences are explicitly written herein for use in the present invention and as basis for potential incorporation into claims below. Also incorporated herein by reference is each AID/APOBEC family member nucleotide and amino acid sequence disclosed in WO2010/113039, including the nucleotide and amino acid sequence of each mutant of an AID/APOBEC family member as though such sequences are explicitly written herein for use in the present invention and as basis for potential incorporation into claims below.

Reference is made to Table 1, which shows the percent identity between various wild-type non-human vertebrate AID amino acid sequences.

TABLE 1
Percent Identities Between Wild-Type AIDs

| | | | | | | | | | | |
|---|---|---|---|---|---|---|---|---|---|---|
| Bos | Canis | Homo | Pan | Danio | Ictalurus | Xenopus | Gallus | Mus | Rattus | Oryctolagus |
| taurus | lupus | sapiens | troglodytes | rerio | punctatus | laevis | gallus | musculus | norvegicus | cuniculus |

| | | | | | | | | | | |
|---|---|---|---|---|---|---|---|---|---|---|
| Bos | 95 | 94 | 94 | 64 | 60 | 67 | 87 | 91 | 93 | 90 |
| taurus | | | | | | | | | | |
| Canis | | 95 | 94 | 65 | 61 | 69 | 90 | 94 | 95 | 93 |
| lupus | | | | | | | | | | |
| Homo | | | 99 | 62 | 58 | 68 | 90 | 92 | 94 | 93 |
| sapiens | | | | | | | | | | |
| Pan | | | | 63 | 58 | 68 | 90 | 93 | 94 | 93 |
| troglodytes | | | | | | | | | | |
| Danio | | | | | 78 | 62 | 61 | 65 | 65 | 62 |
| rerio | | | | | | | | | | |
| Ictalurus | | | | | | 59 | 57 | 59 | 59 | 58 |
| punctatus | | | | | | | | | | |
| Xenopus | | | | | | | 67 | 69 | 68 | 68 |
| laevis | | | | | | | | | | |
| Gallus | | | | | | | | 88 | 87 | 87 |
| gallus | | | | | | | | | | |
| Mus | | | | | | | | | 98 | 92 |
| musculus | | | | | | | | | | |
| Rattus | | | | | | | | | | 93 |
| norvegicus | | | | | | | | | | |

The term “AID homologue” refers to an enzyme that is a member of the APOBEC family, which are (deoxy)cytidine deaminases. Examples of AID homologues are, for example, an APOBEC3 or any APOBEC member listed in table 2 below (or naturally-occurring polymorphic variants thereof).

TABLE 2
AID and AID Homologue NCBI References (Genbank Accession Numbers)

| | Homo sapiens | | Mus musculus | | Rattus norvegicus | |
|---|---|---|---|---|---|---|
| Name | cDNA | Protein | cDNA | Protein | cDNA | Protein |
| AICDA/AID | NM 020661.2 | NP 065712.1 | NM 009645.2 | NP 033775.1 | NM 001100779.1 | NP 001094249.1 |
| APOBEC1 | NM 001644.3 | NP 001635.2 | NM 031159.3; | NP 112436.1; | NM 012907.2 | NP 037039.1 |
| | | | NM 001134391.1 | NP 001127863.1 | | |
| APOBEC2 | NM 006789.3 | NP 006780.1 | NM 009694.3 | NP 033824.1 | NM 001106883.1 | NP 001100353.1 |
| APOBEC3A | NM 145699.3; | NP 663745.1; | NM 001160415.1; | NP 001153887.1; | NM 001033703.1 | NP 001028875.1 |
| | NM 001193289.1 | NP 001180218.1 | NM 030255.3 | NP 084531.2 | | |
| APOBEC3B | NM 004900.3 | NP 004891.3 | | | | |
| APOBEC3C | NM 014508.2 | NP 055323.2 | | | | |
| APOBEC3DE | NM 152426.3 | NP 689639.2 | | | | |
| APOEC3F | NM 145298.5; | NP 660341.2; | | | | |
| | NM 001006666.1 | NP 001006667.1 | | | | |
| APOEC3G | NM 021822.3 | NP 068594.1 | | | | |
| APOEC3H | NM 001166003.1; | NP 001159475.1; | | | | |
| | NM 181773.3; | NP 861438.2; | | | | |
| | NM 001166002.1; | NP 001159474.1; | | | | |
| | NM 001166004.1 | NP 001159476.1 | | | | |
| APOEC4 | NM 203454.2 | NP 982279.1 | NM 001081197.1 | NP 001074666.1 | NM 001017492.1 | NP 001017492.1 |

Table 2 lists possible AID and AID homologues for use in the present invention. Each accession number corresponds to an entry in Genbank. Incorporated herein by reference in its entirety is all the information disclosed in each such Genbank entry, including incorporation of named publications and each AID and APOBEC family member nucleotide and amino acid sequence with or without any non-coding flanking sequence as shown in Genbank (as though explicitly written herein with and without any non-coding region sequence) as though such sequences are explicitly written herein for use in the present invention and as basis for potential incorporation into claims below.

Details of suitable AID mutants are disclosed in WO2010/113039. In one embodiment, the first, second or each expressible gene in the present invention comprises a nucleotide sequence encoding a functional mutant AID whose amino acid sequence differs from the amino acid sequence of a human AID protein (e.g., SEQ ID NO: 12 in the sequence listing herein; or SEQ ID NO: 1 or 2 disclosed in WO2010/113039) by at least one amino acid substitution at a residue selected from the group consisting of residue 34, residue 82, and residue 156, wherein the functional mutant AID protein has at least a 10-fold improvement in activity compared to the human AID protein in a bacterial papillation assay. Details of a suitable bacterial papillation assay are provided in WO2010/113,039, the disclosure pertaining to such assays being explicitly incorporated herein by reference. These residues can be substituted alone, or in any combination. In embodiments where residue 34 lysine (K) is substituted, in one example it is substituted with a glutamic acid (E) or an aspartic acid (D) residue. In embodiments where residue 82 threonine (T) is substituted, in one example it is substituted with an isoleucine (I) or a leucine (L) residue. In embodiments where residue 156 glutamic acid (E) is substituted, in one example it is substituted with a glycine (G) or an alanine (A) residue. When amino acid residue 156 is substituted (either alone, or in combination with a substitution at residue 34 and/or residue 82), in one example there is also an amino acid substitution at one or more of residues 9, 13, 38, 42, 96, 115, 132, 157, 180, 181, 183, 197 and 198. In one example, (a) the amino acid substitution at residue 9 is methionine (M) or lysine (K), (b) the amino acid substitution at residue 13 is phenylalanine (F) or tryptophan (W), (c) the amino acid substitution at residue 38 is glycine (G) or alanine (A), (d) the amino acid substitution at residue 42 is isoleucine (I) or leucine (L), (e) the amino acid substitution at residue 96 is glycine (G) or alanine (A), (f) the amino acid substitution at residue 115 is tyrosine (Y) or tryptophan (W), (g) the amino acid substitution at residue 132 is glutamic acid (E) or aspartic acid (D), (h) the amino acid substitution at residue 180 is isoleucine (I) or alanine (A), (i) the amino acid substitution at residue 181 is methionine (M) or valine (V), (j) the amino acid substitution at residue 183 is isoleucine (I) or proline (P), (k) the amino acid substitution at residue 197 is arginine (R) or lysine (K), (l) the amino acid substitution at residue 198 is valine (V) or leucine (L), and/or (m) the amino acid substitution at residue 157 is threonine (T) or lysine (K). Thus, any one or more of features (a) to (m) is present in this example.

In another embodiment, the nucleic acid molecule encodes a functional AID mutant whose amino acid sequence differs from the amino acid sequence of wild-type AID (e.g., a wild-type human AID) by an amino acid substitution at residue 10 and/or an amino acid substitution at residue 156. These residues can be substituted alone, or in any combination with other substitutions, e.g., any one of substitutions (a) to (m) listed in the paragraph immediately above, in embodiments where amino acid residue 10 (lysine) is substituted, optionally it is substituted with a glutamic acid (E) or aspartic acid (D) residue. In embodiments where residue 156 (glutamic acid) is substituted, optionally it is substituted with a glycine (G) or alanine (A) residue. In embodiments where the amino acids at residues 10 and 156 are substituted, optionally there is an amino acid substitutions at one or more residues selected from 13, 34, 82, 95, 115, 120, 134 and 145. In particular, in one example (a) the amino acid substitution at residue 13 is phenylalanine (F) or tryptophan (W), (b) the amino acid substitution at residue 34 is glutamic acid (E) or aspartic acid (D), (c) the amino acid substitution at residue 82 is isoleucine (I) or leucine (L), (d) the amino acid substitution at residue 95 is serine (S) or leucine (L), (e) the amino acid substitution at residue 115 is tyrosine (Y) or tryptophan (W), (f) the amino acid substitution at residue 120 is arginine (R) or asparagine (N) and/or (g) the amino acid substitution at residue 145 is leucine (L) or isoleucine (I). Thus, any one or more of features (a) to (g) is present in this example.

In another embodiment, the nucleic acid molecule encodes a functional AID mutant whose amino acid sequence differs from the amino acid sequence of wild-type AID (e.g., wild-type human AID) by an amino acid substitution at residue 35 and/or an amino acid substitution at residue 145. The amino acids at residues 35 and/or 145 can be substituted with any suitable amino acid. The amino acid at residue 35optionally is substituted with glycine (G) or alanine (A). The amino acid at residue 145 optionally is substituted with leucine (L) or isoleucine (I).

In another embodiment, the nucleic acid molecule encodes a functional AID mutant whose amino acid sequence differs from the amino acid sequence of wild-type AID (e.g., wild-type human AID) by an amino acid substitution at residue 34 and/or an amino acid substitution at residue 160. The amino acids at residues 34 and 160 can be substituted with any suitable amino acid. The amino acid at residue 34 optionally is substituted with glutamic acid (E) or aspartic acid (D). The amino acid at residue 160optionally is substituted with glutamic acid (E) or aspartic acid (D).

In another embodiment, the nucleic acid molecule encodes a functional AID mutant whose amino acid sequence differs from the amino acid sequence of wild-type AID (e.g., wild-type human AID) by an amino acid substitution at residue 43 and/or an amino acid substitution at residue 120. The amino acids at residues 43 and 120 can be substituted with any suitable amino acid. The amino acid at residue 43optionally is substituted with proline (P). The amino acid at residue 120 optionally is substituted with arginine (R).

In yet another embodiment, the nucleic acid molecule encodes a functional AID mutant whose amino acid sequence differs from the amino acid sequence of wild-type AID (e.g., wild-type human AID) by at least two amino acid substitutions, wherein a substitution is at residue 57 and/or a substitution is at residue 145 or 81. These residues can be substituted alone, or in any combination (e.g., substitution of residues 57 and 145 or substitution of residues 57 and 81). Optionally, the amino acid at residue 57 is substituted with glycine (G) or alanine (A). When the amino acid at residue 145 is substituted, optionally it is substituted with leucine (L) or isoleucine (I). When the amino acid at residue 81 is substituted, optionally it is substituted with tyrosine (Y) or tryptophan (W).

In still another embodiment, the nucleic acid molecule encodes a functional AID mutant whose amino acid sequence differs from the amino acid sequence of wild-type AID (e.g., wild-type human AID) by an amino acid substitution at residue 156 and/or an amino acid substitution at residue 82. The amino acids at residues 156 and 82 can be substituted with any suitable amino acid. The amino acid at residue 156optionally is substituted with glycine (G) or alanine (A). The amino acid at residue 82 optionally is substituted with leucine (L) or isoleucine (I).

In another embodiment, the nucleic acid molecule encodes a functional AID mutant whose amino acid sequence differs from the amino acid sequence of wild-type AID (e.g., wild-type human AID) by an amino acid substitution at residue 156 and/or an amino acid substitution at residue 34. The amino acids at residues 156 and 34 is optionally substituted with any suitable amino acid. The amino acid at residue 156 optionally is substituted with glycine (G) or alanine (A). The amino acid at residue 34 optionally is substituted with glutamic acid (E) or aspartic acid (D).

In another embodiment, the nucleic acid molecule encodes a functional AID mutant whose amino acid sequence differs from the amino acid sequence of wild-type AID (e.g., wild-type human AID) by an amino acid substitution at residue 156 and/or an amino acid substitution at residue 157. The amino acids at residues 156 and 157 can be substituted with any suitable amino acid. The amino acid at residue 156 optionally is substituted with glycine (G) or alanine (A). The amino acid at residue 120 optionally is substituted with arginine (R) or asparagine (N).

In yet another embodiment, the nucleic acid molecule encodes a functional AID mutant whose amino acid sequence differs from the amino acid sequence of wild-type AID (e.g., wild-type human AID) by a amino acid substitution at a residue selected from 10, 82, and 156. These residues can be substituted alone, or in any combination. In one embodiment, the nucleic acid molecule encodes a functional AID mutant whose amino acid sequence differs from the amino acid sequence of wild-type AID (e.g., wild-type human AID) by amino acid substitutions at residues 10, 82, and 156. In embodiments where the amino acids at residues 10, 82, and 156 are substituted, optionally there is a further amino acid substitution at one or more of residues 9, 15, 18, 30, 34, 35, 36, 44, 53, 59, 66, 74, 77, 88, 93, 100, 104, 115, 118, 120 142, 145, 157, 160, 184, 185, 188 and 192. In one embodiment, (a) the amino acid substitution at residue 9 is serine (S), methionine (M), or tryptophan (W), (b) the amino acid substitution at residue 10 is glutamic acid (E) or aspartic acid (D), (c) the amino acid substitution at residue 15 is tyrosine (Y) or leucine (L), (d) the amino acid substitution at residue 18 is alanine (A) or leucine (L), (e) the amino acid substitution at residue 30 is tyrosine (Y) or serine (S), (f) the amino acid substitution at residue 34 is glutamic acid (E) or aspartic acid (D), (g) the amino acid substitution at residue 35 is serine (s) or lysine (K), (h) the amino acid substitution at residue 36 is cysteine (C), (i) the amino acid substitution at residue 44 is arginine (R) or lysine (K), (j) the amino acid substitution at residue 53 is tyrosine (Y) or glutamine (Q), (k) the amino acid substitution at residue 57 is alanine (A) or leucine (L), (l) the amino acid substitution at residue 59 is methionine (M) or alanine (A), (m) the amino acid substitution at residue 66 is threonine (T) or alanine (A), (n) the amino acid substitution at residue 74 is histidine (H) or lysine (K), (o) the amino acid substitution at residue 77 is serine (S) or lysine (K), (p) the amino acid substitution at residue 82 is isoleucine (I) or leucine (L), (q) the amino acid substitution at residue 88 is serine (S) or threonine (T), (r) the amino acid substitution at residue 93 is leucine (L), arginine (R), or lysine (K), (s) the amino acid substitution at residue 100 is glutamic acid (E), tryptophan (W), or phenylalanine F, (t) the amino acid substitution at residue 104 is isoleucine (I) or alanine (A), (u) the amino acid substitution at residue 115 is tyrosine (Y) or leucine (L), (v) the amino acid substitution at residue 118 is glutamic acid (E) or valine (V), (x) the amino acid substitution at residue 120 is arginine (R) or leucine (L), (y) the amino acid substitution at residue 142 is glutamic acid (E) or aspartic acid (D), (z) the amino acid substitution at residue 145 is leucine (L) or tyrosine (Y), (aa) the amino acid substitution at residue 156 is glycine (G) or alanine (A), (bb) the amino acid substitution at residue 157 is glycine (G) or lysine (K), (cc) the amino acid substitution at residue 160 is glutamic acid (E) or aspartic acid (D), (dd) the amino acid substitution at residue 184 is asparagine (N) or glutamine (Q), (ee) the amino acid substitution at residue 185 is glycine (G) or aspartic acid (D), (ff) the amino acid substitution at residue 188 is glycine (G) or glutamic acid (E), and/or (gg) the amino acid substitution at residue 192 is threonine (T) or serine (S). Thus, any one or more of features (a) to (gg) is present in this example.

The functional AID mutant protein can differ from a wild-type AID protein (e.g., human wild-type AID) by any of the amino acid substitutions disclosed herein, alone or in any combination. Alternatively, the functional AID mutant protein can have additional amino acid substitutions as compared to a wild-type AID amino acid sequence (e.g., a human AID amino acid sequence of SEQ ID NO: 1 or SEQ ID NO: 2 disclosed in WO2010/113039, which sequences are incorporated by reference herein). For example, a functional AID mutant protein has one, two, three or any other combination of, the following amino acid substitutions with respect to said SEQ ID NO: 1 or SEQ ID NO: 2 disclosed in WO2010/113039: N7K, R8Q, Q14H, R25H, Y48H, N52S, H156R, R158K, L198A, R9K, G100W, A138G, S173T, T195I, F42C, A138G, H156R, L198F M6K, K10Q, A39P, N52A, E11SD, K10L, Q14N, N52M, D67A, G100A, V135A, Y145F, R171H, Q175K, R194K, insertion of K after residue 118, and D119E.

The invention also includes the use of first and/or second expressible genes encoding a functional AID mutant comprising a C-terminal truncation mutation. The generation of a C-terminal truncation mutation is within the ordinary skill in the art. For example, the C-terminal truncation mutation can be generated by the insertion of a stop codon at or distal to residue 181 of the human AID amino acid sequence.

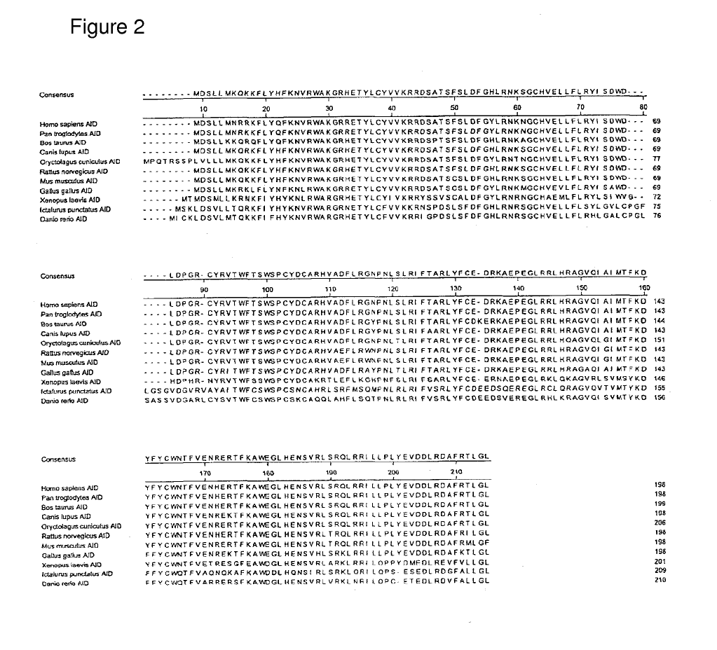

Examples of preferred amino acid substitutions that produce functional AID mutant proteins in the context of the invention are illustrated in FIG. 2 of WO2010/113039, which disclosure is incorporated herein by reference.

In the context of the invention, a functional AID mutant also includes a nucleic acid sequence encoding a wild-type AID protein (e.g., wild-type human AID) in which a portion of the nucleic acid sequence is deleted and replaced with a nucleic acid sequence from an AID homologue (e.g., Apobec-1, Apobec3C or Apobec3G). In this respect, the human APOBEC3 proteins, like human AID, are able to deaminate cytosine (C) in DMA but, whereas AID prefers to target C residues flanked by a 5′-flanking purine, the APOBEC3s prefer a 5′-pyrimidine flank, with individual APOBEC3s differing with regard to the specific 5′-flanking nucleotide preference. Comparison of human APOBEC3 gene sequences suggests that a stretch of around eight amino acids located about 60 residues from the carboxy terminal end of the protein domain plays an important role in determining this flanking nucleotide preference. In view of the crystal structure of APOBEC2 and the crystal structure of the TadA tRNA-adenosine deaminase in complex with an oligonucleotide substrate, this 60-amino acid sequence in both AID and APOBEC3s likely forms a contact with the DMA substrate. Therefore, in one embodiment the first and/or second expressible gene encodes a functional AID mutant that comprises a nucleic acid sequence encoding a wild-type AID protein (e.g., wild-type human AID) in which amino acid residues 115-223 are removed and replaced with the corresponding sequence from APOBEC3 proteins (e.g., APOBEC3C, APOBec3F, and APOBEC3G).

Functional AID mutants are deoxycytidine or cytidine deaminases, ie, they are RNA or DNA editing enzymes that mediate the deamination of cytosine to uracil in nucleic acid sequences (see, eg, Conticello, Genome Biol. 2008; 9(6):229. Epub 2008 Jun. 17. Review; Conticello et al, Mol Biol Evol, 22: 367-377 (2005); and U.S. Pat. No. 6,815,194).

Optionally, for each AID mutant or AID homologue mutant in any configuration of the invention, the mutant retains a wild-type Hot Spot Recognition Loop. Reference is made to Kohli, R M et al, “A Portable Hot Spot Recognition loop Transfers Sequence Preference from APOBEC Family Member to Activation-induced Cytidine Deaminase”, (2009) J Biol. Chem. 284: 22898-22904; and to Holden, L G et al, “Crystal structure of the anti-viral APOBEC3G catalytic domain and functional implications”, (2008) Nature. 456:121-124, the disclosures of which are incorporated herein by reference, including the incorporation of Hot Spot Recognition Loop sequences as disclosed in these publications as though they are written explicitly herein as individual loop sequences (without flanking sequences) for use in the present invention and potential inclusion in claims herein. Thus, in one embodiment of the invention, the mutant retains a Hot Spot Recognition Loop (e.g., as disclosed in Kohli, R M et al) or an Active-Site Loop (e.g., as disclosed in Holden, L G et al).

The terms “functional mutant of AID,” “functional AID mutant,” or “functional mutant AID protein.” each refer to a mutant AID protein which retains all or part of the biological activity of a wild-type AID and/or which exhibits increased biological activity as compared to a wild-type AID protein. The biological activity of a wild-type AID that is retained in all or part includes, but is not limited to, the deamination of cytosine to uracil within a DNA sequence, papillation in a bacterial mutagenesis assay, somatic hypermutation of a target gene, and immunoglobulin class switching. A mutant AID protein can retain any part of the biological activity of a wild-type AID protein. Desirably, the mutant AID protein has at least 75% (e.g., 75%, 80%, 90% or more) of the biological activity of wild-type AID. Optionally, the mutant AID protein has at least 90% (e.g., 90%, 95%, 100%, 110%, 120%, 130%, 140%, 150%, 175% or 200% or more) of the biological activity of wild-type AID, eg, human wild-type AID.

In a preferred embodiment, the mutant AID protein exhibits increased biological activity as compared to a wild-type AID protein. In this respect, the functional AID mutant has at least a 10-fold improvement in activity compared to a wild-type AID protein as measured by a bacterial papillation assay. Bacterial papillation assays are known in the art as useful for screening fort, coli mutants that are defective in some aspect of DNA repair (Nghiem et al., Proc. Natl. Acad. Sci. USA, 85: 2709-2713 (1988) and Ruiz et al., J. Bacteriol., 175: 4985-4989 (1993)). The bacterial papillation assay can employ Escherichia coli CC102 cells harbouring a missense mutation within the lacZ gene. E. coli CC102 cells give rise to white colonies on MacConkey-lactose plates. Within such white colonies, a small number of red microcolonies, or “papilli,” can often be discerned (typically 0-2 per colony), which reflect spontaneously-arising La [symbol] revertants. Bacterial clones which exhibit an elevated frequency of spontaneous mutation (i.e., “mutator clones”) can be identified by virtue of an increased number of papilli. Bacterial papillation assays can be used to screen for functional AID mutants having increased activity as compared to wild-type AID. Bacterial papillation assays are described in detail in the Examples of WO2010/113039 the disclosure of which assays is incorporated herein by reference.

In one embodiment, the functional AID mutant has at least a 10-fold (e.g., 10-fold, 30-fold, 50-fold or more) improvement in activity compared to the wild-type AID protein in a bacterial papillation assay. Preferably, the functional AID mutant has at least a 100-fold (e.g., 100-fold, 200-fold, 300-fold or more) improvement in activity compared to wild-type AID. More preferably, the functional AID mutant has at least a 400-fold (e.g., 400-fold, 500-fold, 1000-fold or more) improvement in activity compared to wild-type AID.

One of ordinary skill in the art will appreciate that although there is a high degree of homology among the vertebrate AID proteins, there is a variable number of amino acid substitutions, deletions, and insertions in each of the vertebrate AID protein relative to human AID. As such, the present invention encompasses embodiments in which the first and/or second expressible gene encodes mutant AID protein with mutations described herein or in WO2010/113039 when incorporated at the analogous position of any vertebrate AID protein. One of ordinary skill in the art can determine the analogous position in any vertebrate AID protein by performing a sequence alignment of the homologous vertebrate AID protein with that of a human AID using any computer based alignment program known in the art (e.g., BLAST or ClustalW2).

Table 3 shows nucleotide coordinates on human chromosome 12 defining regions comprising sequences that encode human AID.

TABLE 3
Human AID-Encoding Sequences

| | | | | |
|---|---|---|---|---|
| Homo | AICDA | human genome assembly | 12 | 8646028-8656706 |
| sapiens | | Human Genome Assembly | 12 | 8646029-8656706 |
| | | Build 36.2 | | |
| | | Cytogenetic | 12 | p13 |
| | | Human Genome Assembly | 12 | 8537559-8548246 |
| | | HuRef | | |
| | | Human Genome Assembly | 12 | 8754762-8765442 |
| | | GRCh37 | | |
| | | Human Celera Assembly | 12 | 10292343-10303027 |

In one aspect of any configuration or aspect of the invention, reference to a human AID is to be read as reference to an AID encoded by a nucleotide sequence from (i) position 8646028 to 8656706 of human chromosome 12; (ii) position 8646029 to 8656706 of human chromosome 12; (iii) position 8537559 to 8548246 of human chromosome 12; (iv) position 8754762 to 8765442 of human chromosome 12; or (v) position 10292343 to 10303027 of human chromosome 12. In one embodiment of any configuration or aspect of the invention, reference to a human AID is to be read as reference to an AID encoded by region p13 of human chromosome 12.

Optimisation of AID/APOBEC Family Member Sequences

Optionally, at least one V, D and/or J region sequence in the transgene has been codon-optimised for somatic hypermutation (SHM). In one embodiment of the vertebrate or cell of any aspect of the present invention, at least one V, D and/or J region sequence in the transgene has been codon-optimised for AID or an AID homologue, optionally wherein the V, D and/or J sequence has been changed to include a SHM hot spot selected from the group consisting of DGYW, WRC, WRCY, WRCH, RGYW, AGY,TAC, WGCW, wherein W=A or T, Y=C or T, D=A, G or T, H=A or C or T, and R=A or G.

For example, codon optimisation may be effected to increase the number of somatic hypermutation (SHM) motifs. As used herein, “somatic hypermutation” or “SHM” refers to the mutation of a polynucleotide sequence initiated by, or associated with the action of AID (e.g., a wild-type AID or functional AID mutant) or an AID homologue on that polynucleotide sequence. The term is intended to include mutagenesis that occurs as a consequence of the error prone repair of the initial lesion, including mutagenesis mediated by the mismatch repair machinery and related enzymes. The term “substrate for SHM” refers to a polynucleotide sequence which is acted upon by AID (e.g., a wild-type AID or functional AID mutant) or an AID homologue to effect a change in the sequence of the polynucleotide sequence. As used herein, the term “SHM hot spot” or “hot spot” refers to a polynucleotide sequence, or motif, of 3-6 nucleotides that exhibits an increased tendency to undergo somatic hypermutation, as determined via a statistical analysis of SHM mutations in antibody genes. A relative ranking of various motifs for SHM as well as canonical hot spots n antibody genes are described in US2009/0075378 and International Patent Application Publication WO2008/103475 (the disclosures of which are incorporated herein by reference). The term “somatic hypermutation motif” or “SHM motif” refers to a polynucleotide sequence that includes, or can be altered to include, one or more hot spots, and which encodes a defined set of amino acids. SHM motifs can be of any size, but are conveniently based around polynucleotides of about 2 to about 20 nucleotides in size, or from about 3 to about 9 nucleotides in size. SHM motifs can include any combination of hot spots. The terms “preferred hot spot SHM codon,” “preferred hot spot SHM motif,” “preferred SHM hot spot codon” and “preferred SHM hot spot motif,” all refer to a codon including, but not limited to codons AAC, TAC, TAT, AGT, or AGC. Such sequences may be potentially embedded within the context of a larger SHM motif, recruits SHM mediated mutagenesis and generates targeted amino acid diversity at that codon. As used herein, a nucleic acid sequence has been “optimized for SHM” if the nucleic acid sequence, or a portion thereof has been altered to increase or decrease the frequency and/or location of hot spots within the nucleic acid sequence. A nucleic acid sequence that has been made “susceptible to SHM” if the nucleic acid sequence, or a portion thereof, has been altered to increase the frequency and/or location of hot spots within the nucleic acid sequence, in general, a sequence can be prepared that has a greater propensity to undergo SHM mediated mutagenesis by altering the codon usage, and/or the amino acids encoded by nucleic acid sequence. Further detail is found in WO2008/103475.

Optimization of a nucleic acid sequence or nucleotide sequence refers to modifying about 1%, about 2%, about 3%, about 4%, about 5%, about 10%, about 20%, about 25%, about 50%, about 75%, about 90%, about 95%, about 96%, about 97%, about 98%, about 99%, about 100%, or any range therein, of the nucleotides in the sequence. Optimization of a nucleic acid sequence or nucleotide sequence also refers to modifying about 1, about 2, about 3, about 4, about 5, about 10, about 20, about 25, about 50, about 75, about 90, about 95, about 96, about 97, about 98, about 99, about 100, about 200, about 300, about 400, about 500, about 750, about 1000, about 1500, about 2000, about 2500, about 3000 or more, or any range therein, of the nucleotides in the nucleic acid sequence such that some or all of the nucleotides are optimized for SHM-mediated mutagenesis. Increasing the frequency (density) of hot spots refers to increasing about 1%, about 2%, about 3%, about 4%, about 5%, about 10%, about 20%, about 25%, about 50%, about 75%, about 90%, about 95%, about 96%, about 97%, about 98%, about 99%, about 100%, or any range therein, of the hot spots in a nucleic acid sequence.

The position or reading frame of a hot spot is also a factor governing whether SHM-mediated mutagenesis that can result in a mutation that is silent with regards to the resulting amino acid sequence, or causes conservative, semi-conservative or non conservative changes at the amino acid level. The design parameters can be manipulated to further enhance the relative susceptibility of a nucleotide sequence to SHM. Thus both the degree of SHM recruitment and the reading frame of the motif are considered in the design of SHM susceptible nucleic acid sequences. More details are given in WO2010/113039, US2009/0075378 and International Patent Application Publication WO2008/103475.

Localisation of Genes in Mouse and Mouse Cell Genomes

In one embodiment, the first, the second, or both expressible AID or AID homologue genes are present on a copy of chromosome 6 when the vertebrate is a mouse or the vertebrate cell is a mouse. The position of the AID nucleotide sequence on chromosome 6 has been mapped for C57BL/6J mouse. This position is coordinate 122503819 to coordinate 122514198, which is in region 6F2 of chromosome 6 in mouse.

In certain embodiments, the first and/or second expressible AID or homologue sequences are placed under the control of endogenous control elements which regulate the expression and activity of endogenous AID. This is advantageous for enabling expression and activity of the inserted AID or homologue in a way that harnesses beneficial somatic hypermutation while minimising unwanted over-activity of the AID or the homologue and associated events such as possible chromosome translocation (see, eg, R Maul & P Gearhart, Advances in immunology, 2010, volume 105, Chapter 6 (pp 159-191): AID and Somatic Hypermutation).

Thus, in one embodiment of any configuration of the invention,
a) the vertebrate is a mouse; or
b) the cell is a mouse cell; and
c) the first expressible gene has been constructed by insertion of an AID or AID homologue nucleotide sequence in the mouse or cell genome between (i) coordinates 122503500 and 122514700, in one embodiment between coordinates 122503818 and 122514199, of a first chromosome 6 when the mouse is a C57BL/6J mouse strain, or (ii) between equivalent coordinates on a first chromosome 6 when the mouse is a strain other than C57BL/6J; and
d) optionally no nucleotides of the endogenous AID nucleotide sequence immediately flank the inserted AID or homologue nucleotide sequence in said genome. The endogenous AID nucleotide sequence is comprised by the region from coordinate 122503818 to coordinate 122514199 in a C57BL/6J mouse strain or equivalent coordinates when the mouse is a strain other than C57BL/6J.

Additionally or alternatively, in one embodiment, the second expressible gene is inserted in the other copy of chromosome 6 in the mouse or mouse cell. In one aspect of any configuration of the invention,
a) the vertebrate is a mouse; or
b) the cell is a mouse cell; and
c) the second expressible gene has been constructed by insertion of an AID or AID homologue nucleotide sequence in the mouse or cell genome between (i) coordinates 122503500 and 122514700, in one embodiment between coordinates 122503818 and 122514199, of a first chromosome 6 when the mouse is a C57BL/6J mouse strain, or (ii) between equivalent coordinates on a first chromosome 6 when the mouse is a strain other than C57BL/6J; and
d) optionally no nucleotides of the endogenous AID nucleotide sequence immediately flank the inserted AID or homologue nucleotide sequence in said genome. The endogenous AID nucleotide sequence is comprised by the region from coordinate 122503818 to coordinate 122514199 in a C57BL/6J mouse strain or equivalent coordinates when the mouse is a strain other than C57BL/6J.

Thus, a possible combination for any configuration of the invention is that one or both of the first and second expressible genes is on a chromosome 6 (when the vertebrate is a mouse or the cell is a mouse cell) and operably linked, eg, in germline configuration, with one or more endogenous control elements that controls the expression and/or activity of endogenous AID in a wild-type mouse or mouse cell.

In one aspect,
a) the vertebrate is a mouse; or
b) the vertebrate cell is a mouse cell; and
c) the AID encoded by the first expressible gene is AID endogenous to the mouse or mouse cell; and
d) the second expressible gene comprises an exogenous AID or AID homologue nucleotide sequence;
e) wherein one or both of the first and second expressible genes is on a chromosome 6 and operably linked, eg, in germline configuration, with one or more endogenous control elements that controls the expression and/or activity of endogenous AID in a wild-type mouse or mouse cell.

Each exogenous AID or homologue is functional and is, for example, a human or mutant AID or AID homologue wherein the amino acid sequence of the mutant is at least 95% (or at least 96, 97, 98 or 99%) identical to the amino acid sequence of a human or mouse AID/APOBEC family member. For example, the amino acid sequence of the mutant is at least 95% (or at least 96, 97, 98 or 99%) identical to the amino acid sequence of a human or mouse AID, APOBEC1, APOBEC3C, APOBEC3F or APOBEC3G. Such mutants function as (deoxy)cytidine deaminases.

In another aspect (relating to the second configuration of the invention), the vertebrate is a mouse; or
a) the vertebrate cell is a mouse cell; and
b) the first expressible gene comprises an exogenous AID or AID homologue nucleotide sequence; and
c) the second expressible gene comprises an exogenous AID or AID homologue nucleotide sequence;
d) wherein one or both of the first and second expressible genes is on a chromosome 6 and operably linked, eg, in germline configuration, with one or more endogenous control elements that controls the expression and/or activity of endogenous AID in a wild-type mouse or mouse cell.

Each exogenous AID or homologue is functional and is, for example, a human or mutant AID or AID homologue wherein the amino acid sequence of the mutant is at least 95% (or at least 96, 97 or 99%) identical to the amino acid sequence of a human AID/APOBEC family member. For example, the amino acid sequence of the mutant is at least 95% (or at least 96, 97, 98 or 99%) identical to the amino acid sequence of a human or mouse AID, APOBEC1, APOBEC3C, APOBEC3F or APOBEC3G. Such mutants function as (deoxy)cytidine deaminases.

Thus, in one embodiment of any configuration of the invention,
a) the vertebrate is a mouse; or
b) the cell is a mouse cell; and
c) the first and/or second expressible genes have been constructed by insertion of an AID or AID homologue nucleotide sequence in the mouse or cell genome in region 6F2 of a respective chromosome 6, optionally in operable linkage with one or more endogenous control elements that controls the expression and/or activity of endogenous AID in a wild-type mouse or mouse cell.

Localisation of Genes in Rat and Rat Cell Genomes

In one embodiment, the first, the second, or both expressible AID or AID homologue genes are present on a copy of chromosome 4 when the vertebrate is a rat or the vertebrate cell is a rat. The position of the AID nucleotide sequence on chromosome 4 has been mapped for Rattus norvegicus. This position is in a region defined by coordinate 144595276 to coordinate 159017501 (e.g., in a region defined by coordinate 159257307 to coordinate 159260429; or coordinate 144595276 to coordinate 144605030; or coordinate 159006328 to coordinate 159017501), which is in region q42 of chromosome 4 in rat.

In the following embodiments, the first and/or second expressible AID or homologue sequences are placed under the control of endogenous control elements which regulate the expression and activity of endogenous AID. This is advantageous for enabling expression and activity of the inserted AID or homologue in a way that harnesses beneficial somatic hypermutation while minimising unwanted over-activity of the AID or the homologue and associated events such as possible chromosome translocation.

Thus, in one embodiment of any configuration of the invention,
a) the vertebrate is a rat; or
b) the cell is a rat cell; and
c) the first expressible gene has been constructed by insertion of an AID or AID homologue nucleotide sequence in the rat or cell genome between (i) coordinates 144595276 and 159017501, in one embodiment between coordinates 159257307 and 159260429, in an alternative embodiment between coordinates 144595276 and 144605030, in an alternative embodiment between coordinates 159006328 and 159017501, of a first chromosome 4 when the rat is a Rattus norvegicus rat strain, or (ii) between equivalent coordinates on a first chromosome 4 when the rat is a strain other than Rattus norvegicus; and
d) optionally no nucleotides of the endogenous AID nucleotide sequence immediately flank the inserted AID or homologue nucleotide sequence in said genome. The wild-type AID nucleotide sequence is comprised by the region from coordinate 159257307 to coordinate 159260429; or coordinate 144595276 to coordinate 144605030; or coordinate 159006328 to coordinate 159017501 in a Rattus norvegicus rat strain or equivalent coordinates when the rat is a strain other than Rattus norvegicus.

Additionally or alternatively, in one embodiment, the second expressible gene is inserted in the other copy of chromosome 4 in the rat or rat cell. In one aspect of any configuration of the invention,
a) the vertebrate is a rat; or
b) the cell is a rat cell; and
c) the second expressible gene has been constructed by insertion of an AID or AID homologue nucleotide sequence in the rat or cell genome between (i) coordinates 144595276 and 159017501, in one embodiment between coordinates 159257307 and 159260429, in an alternative embodiment between coordinates 144595276 and 144605030, in an alternative embodiment between coordinates 159006328 and 159017501, of a first chromosome 4 when the rat is a Rattus norvegicus rat strain, or (ii) between equivalent coordinates on a first chromosome 4 when the rat is a strain other than Rattus norvegicus; and
d) optionally no nucleotides of the endogenous AID nucleotide sequence immediately flank the inserted AID or homologue nucleotide sequence in said genome. The wild-type AID nucleotide sequence is comprised by the region from coordinate 159257307 to coordinate 159260429; or coordinate 144595276 to coordinate 144605030; or coordinate 159006328 to coordinate 159017501 in a Rattus norvegicus rat strain or equivalent coordinates when the rat is a strain other than Rattus norvegicus.

Thus, a possible combination for any configuration of the invention is that one or both of the first and second expressible genes is on a chromosome 4 (when the vertebrate is a rat or the cell is a rat cell) and operably linked, eg, in germline configuration, with one or more endogenous control elements that controls the expression and/or activity of endogenous AID in a wild-type rat or rat cell.

In one aspect,
a) the vertebrate is a rat; or
b) the vertebrate cell is a rat cell; and
c) the AID encoded by the first expressible gene is AID endogenous to the rat or rat cell; and
d) the second expressible gene comprises an exogenous AID or AID homologue nucleotide sequence;
e) wherein one or both of the first and second expressible genes is on a chromosome 4 and operably linked, eg, in germline configuration, with one or more endogenous control elements that controls the expression and/or activity of endogenous AID in a wild-type rat or rat cell.

Each exogenous AID or homologue is functional and is, for example, a human or mutant AID or AID homologue wherein the amino acid sequence of the mutant is at least 95% (or at least 96, 97, 98 or 99%) identical to the amino acid sequence of a human or rat AID/APOBEC family member. For example, the amino acid sequence of the mutant is at least 95% (or at least 96, 97, 98 or 99%) identical to the amino acid sequence of a human or rat AID, APOBEC1, APOBEC3C, APOBEC3F or APOBEC3G. Such mutants function as (deoxy)cytidine deaminases.

In another aspect (relating to the second configuration of the invention),
a) the vertebrate is a rat; or
b) the vertebrate cell is a rat cell; and
c) the first expressible gene comprises an exogenous AID or AID homologue nucleotide sequence; and
d) the second expressible gene comprises an exogenous AID or AID homologue nucleotide sequence;
e) wherein one or both of the first and second expressible genes is on a chromosome 4 and operably linked, eg, in germline configuration, with one or more endogenous control elements that controls the expression and/or activity of endogenous AID in a wild-type rat or rat cell.

Each exogenous AID or homologue is functional and is, for example, a human or mutant AID or AID homologue wherein the amino acid sequence of the mutant is at least 95% (or at least 96, 97, 98 or 99%) identical to the amino acid sequence of a human AID/APOBEC family member. For example, the amino acid sequence of the mutant is at least 95% (or at least 96, 97, 98 or 99%) identical to the amino acid sequence of human or rat AID, APOBEC1, APOBEC3C, APOBEC3F or APOBEC3G. Such mutants function as (deoxy)cytidine deaminases.

Thus, in one embodiment of any configuration of the invention,
a) the vertebrate is a rat; or
b) the cell is a rat cell; and
c) the first and/or second expressible genes have been constructed by insertion of an AID or AID homologue nucleotide sequence in the rat or cell genome in region q42 of a respective chromosome 4, optionally in operable linkage with one or more endogenous control elements that controls the expression and/or activity of endogenous AID in a wild-type rat or rat cell.

Inducible AID or AID Homologue Genes

In one embodiment of any configuration or aspect of the invention, the expression of one, both or all AIDs, AID homologues or chimaeric AIDs is inducible. Suitable systems for inducible expression of genes in vertebrate cells will be known to the skilled person, for example, use of a positive/negative regulatory tet system or an ecdysone receptor-inducible system as disclosed at page 16 of WO03/061363 (the disclosure of which is incorporated herein in by reference).

Chimaeric AIDs or AID Homologues

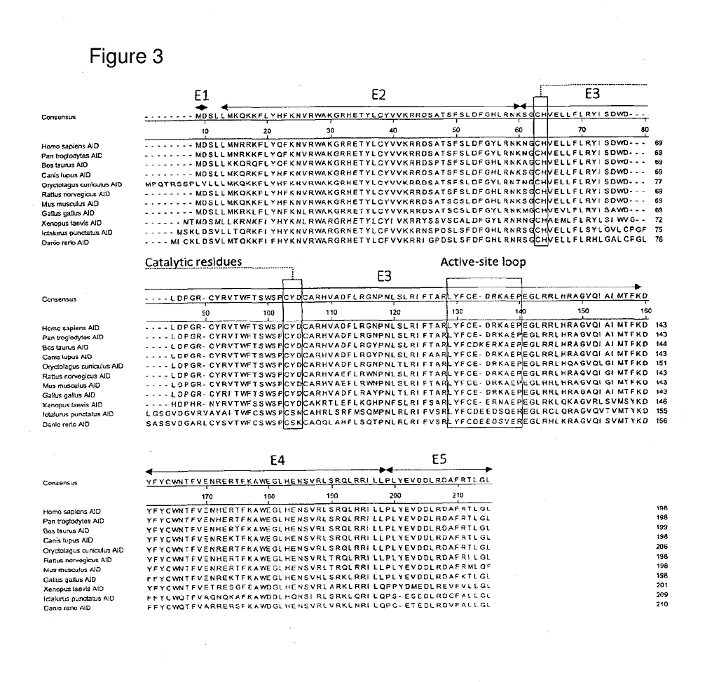

Crystal structural analysis of the AID homologue, APOBEC3G revealed an active-site loop (hot-spot recognition loop) that is directly involved in substrate binding (Holden, L G et al Nature, 456: 121-124). Grafting the loop from APOBEC3G or APOBEC3F into the AID scaffold alters the mutational spectrum toward that of the two donor enzymes (Kohli, R M et al Journal of Biological Chemistry, 284:22898-22904; Carpenter, M A et al DMA Repair, 9:579-587; Wang, M et al Journal of Experimental Medicine, 207: 141-153). These studies highlight the crucial role of the active-site loop in AID for DNA sequence preference in hypermutation. The sequence encoding the active-site loop is within exon 3 of the AID gene (see FIG. 3). In addition, the sequence encoding the two catalytic residues is in exon 3 as well. These observations point out that replacing exon 3 or the active-site loop-encoding sequence to the corresponding region from orthologues or homologues in the genome will generate mutant AIDs with a new and different mutational spectrum from that of the wild-type AID. And expression of such a mutant in one allele and the wild-type AID in the other allele in a genome of a non-human vertebrate is likely to provide a broader mutational spectrum of SHM and CSR, and produce more antibody diversity.

Thus, in one embodiment, the invention uses an expressible gene that encodes a functional AID mutant in which the mutant is a chimaeric protein comprising AID sequences from two or more species. For example, the chimaeric AID gene is mouse or rat AID gene in which exon 3 sequence been replaced by a (i) corresponding sequence (e.g., the entire exon 3 sequence or an active-site loop and/or a catalytic residue-encoding sequence) from an AID gene of a different species (e.g., human, reptile, fish, bird, catfish, zebrafish Xenopus or chicken AID gene); or (ii) corresponding sequence (e.g., the entire exon 3 sequence; or an active-site loop and/or a catalytic residue-encoding sequence) from an APOBEC family member (as defined above) gene of a different species (e.g., human, reptile, fish, bird, catfish, zebrafish Xenopus or chicken AID) or from the same species (mouse or rat APOBEC member gene).

Thus, in another embodiment, the invention uses an expressible gene that encodes a functional AID homologue gene in which the homologue is a chimaeric protein comprising APOBEC family member nucleotide sequences from two or more species or an APOBEC family member gene sequence from one species and an AID nucleotide sequence from another species. For example, the homologue is mouse or rat APOBEC in which exon 3 sequence been replaced in the gene by a (i) corresponding sequence (e.g., the entire exon 3 sequence or an active-site loop and/or a catalytic residue-encoding sequence) from an APOBEC of a different species (e.g., human, reptile, fish, bird, catfish, zebrafish Xenopus or chicken AID); or (ii) corresponding sequence (e.g., the entire exon 3 sequence; or an active-site loop and/or a catalytic residue) from an AID of a different species (e.g., human, reptile, fish, bird, catfish, zebrafish Xenopus or chicken AID) or from the same species (mouse or human APOBEC member gene) or from the same species (mouse or rat AID gene).

Thus in any aspect herein of the first configuration of the invention, “AID” can be read to include a chimaeric AID as described above, eg,
(a) wherein the first expressible gene encodes a chimaeric AID, the gene being a mouse or rat AID gene in which exon 3 has been replaced by an exon 3 sequence from an AID gene selected from a fish, a reptile, a chicken, Xenopus, catfish, zebrafish or human AID gene. Advantageously, the mouse or rat AID gene includes the intervening sequences between exons, inclusion of such intervening sequences may be beneficial in the control of expression of the gene. Thus, endogenous (mouse or rat) control can be exerted on the expression of a chimaeric protein that includes foreign AID sequences/activity. For example, where the non-human vertebrate of the invention is a mouse or rat (or the cell of the invention is a mouse or rat cell), the chimaeric AID is encoded by a mouse or rat gene that is endogenous to the vertebrate, but which has exon 3 replaced by the foreign exon 3. This provides for expression control by intervening sequences that are endogenous to the vertebrate (vertebrate cell);
   or
(b) wherein the first expressible gene encodes a chimaeric AID, the gene being a mouse or rat AID gene in which an active-site-encoding loop sequence has been replaced by a corresponding active-site-encoding loop sequence from an AID gene selected from a fish, a reptile, Xenopus, catfish or zebrafish AID gene. Advantageously, the mouse or rat AID gene includes the intervening sequences between exons. Inclusion of such intervening sequences may be beneficial in the control of expression of the gene. Thus, endogenous (mouse or rat) control can be exerted on the expression of a chimaeric protein that includes foreign AID sequences/activity. For example, where the non-human vertebrate of the invention is a mouse or rat (or the cell of the invention is a mouse or rat cell), the chimaeric AID is encoded by a mouse or rat gene that is endogenous to the vertebrate, but which has an active-site-encoding loop sequence replaced by a corresponding active-site-encoding loop sequence from the foreign AID gene. This provides for expression control by intervening sequences that are endogenous to the vertebrate (vertebrate cell);
   and
(c) optionally wherein
   (i) the vertebrate is a mouse (or vertebrate cell is a mouse cell) and the chimaeric AID gene is a mouse AID gene according to (a) or (b), the vertebrate or cell comprises an additional AID gene, wherein said additional AID gene is a wild-type mouse AID gene (e.g., a wild-type mouse AID gene that is endogenous to the vertebrate or cell); or
   (ii) the vertebrate is a rat (or vertebrate cell is a rat cell) and the chimaeric AID gene is a rat AID gene according to (a) or (b), the vertebrate or cell comprises an additional AID gene, wherein said additional AID gene is a wild-type rat AID gene (e.g., a wild-type rat AID gene that is endogenous to the vertebrate or cell.

Option (c) is beneficial for providing enhanced AID diversity by provision of one AID allele that encodes a chimaeric AID and a second AID allele that encodes a second, different, AID being wild-type and with its own SHM and CSR-creating spectrum.

The invention provides a chimaeric AID comprising a mouse or rat AID (e.g., a wild-type AID) in which the active-site loop has been replaced with a foreign active-site loop, optionally a human, chicken, bird, fish, reptile, Xenopus, catfish or zebrafish AID active-site loop. In one embodiment, the mouse or rat AID (with the exception of the foreign loop) is an AID that is endogenous to the non-human vertebrate or cell of the invention and the chimaeric AID is encoded by a gene that is integrated into the genome of said vertebrate or cell (ie, mouse, rat, mouse cell or rat cell).

The invention provides a nucleic acid comprising a nucleotide sequence encoding the chimaeric AID of the invention. Optionally, the nucleotide sequence is provided as a gene sequence with exons and intervening sequences. Optionally, one or more gene control regions upstream or downstream of the AID gene is included.

The invention provides a nucleic acid comprising a nucleotide sequence encoding a chimaeric AID, wherein the nucleotide sequence comprises a nucleotide sequence encoding mouse or rat AID wherein exon 3 has been replaced with an exon 3 nucleotide sequence selected from a human, chicken, bird, fish, reptile, Xenopus, catfish or zebrafish AID gene exon 3 nucleotide sequence. Optionally, the nucleotide sequence is provided as a gene sequence with exons and intervening sequences. Optionally, one or more gene control regions upstream or downstream of the AID gene is included.

The invention provides a nucleic acid comprising a nucleotide sequence encoding a chimaeric AID, wherein the nucleotide sequence comprises a nucleotide sequence encoding mouse or rat AID wherein the active-site loop-encoding nucleotide sequence has been replaced with an active-site loop-encoding nucleotide sequence selected from a human, chicken, bird, fish, reptile, Xenopus, catfish or zebrafish AID active-site loop-encoding nucleotide sequence. Optionally, the nucleotide sequence is provided as a gene sequence with exons and intervening sequences. Optionally, one or more gene control regions upstream or downstream of the AID gene is included.

The invention provides a chimaeric AID comprising an amino acid sequence selected from the group consisting of SEQ ID NO: 54, 56 and 58, or a sequence that is at least 70, 75, 80, 85, 90, 91, 92, 93, 94, 95, 96, 97, 98 or 99% identical thereto (or 100% identical thereto).

The invention provides a nucleic acid comprising a nucleotide sequence encoding a chimaeric AID, wherein the nucleotide sequence is selected from the group consisting of SEQ ID NO: 53, 55 and 57, or a sequence that is at least 70, 75, 80, 85, 90, 91, 92, 93, 94, 95, 96, 97, 98 or 99% identical thereto (or 100% identical thereto).

The invention provides a nucleotide sequence encoding a chimaeric AID of the invention when integrated into the genome of a non-human vertebrate mammal or the genome of a non-human vertebrate cell, optionally wherein said genome further comprises an endogenous gene encoding a wild-type AID or a gene encoding an AID, chimaeric AID or an AID homologue. In one embodiment, the vertebrate is a mouse or rat; or the cell is a mouse cell or rat cell. For example, the vertebrate is a mouse, the wild-type AID is endogenous to the mouse and the chimaeric AID is also the AID that is endogenous to the mouse with the exception that the active-site loop has been replaced by the foreign loop or wherein the amino acid sequence encoded by exon 3 has been replaced by a sequence encoded by the foreign exon 3.

The chimaeric AIDs of the invention are (deoxy) cytidine deaminases.

### REFERENCES

1. Local sequence targeting in the AID/APOBEC family differentially impacts retroviral restriction and antibody diversification.
   Kohli R M, Maul R W, Guminski A F, McClure R L, Gajula K S, Saribasak H, McMahon M A, Siliciano R F, Gearhart P J, Stivers J T.
   J Biol. Chem. 2010 Oct. 6.
2. AID and somatic hypermutation.
   Maul R W, Gearhart P J.
   Adv Immunol. 2010; 105:159-91. Review.
3. Determinants of sequence-specificity within human AID and APOBEC3G.
   Carpenter M A, Rajagurubandara E, Wijesinghe P, Bhagwat A S.
   DNA Repair (Amst). 2010 May 4; 9(5):579-87. Epub 2010 Mar. 24.
4. Altering the spectrum of immunoglobulin V gene somatic hypermutation by modifying the active site of AID.
   Wang M, Rada C, Neuberger M S.
   Exp Med. 2010 Jan. 18; 207(1):141-53. Epub 2010 Jan. 4.
5. Haploinsufficiency of activation-induced deaminase for antibody diversification and chromosome translocations both in vitro and in vivo.
   Sernandez I V, de Yébenes V G, Dorsett Y, Ramiro A R.
   PLoS One. 2008; 3(12):e3927. Epub 2008 Dec. 12.
6. A portable hot spot recognition loop transfers sequence preferences from APOBEC family members to activation-induced cytidine deaminase.
   Kohli R M, Abrams S R, Gajula K S, Maul R W, Gearhart P J, Stivers J T.
   J Biol. Chem. 2009 Aug. 21; 284(34):22898-904.
7. Crystal structure of the anti-viral APOBEC3G catalytic domain and functional implications.
   Holden L G, Prochnow C, Chang Y P, Bransteitter R, Chelico L, Sen U, Stevens R C, Goodman M F, Chen X S.
   Nature. 2008 Nov. 6; 456(7218):121-4. Epub 2008 Oct. 12.
8. Activation-induced cytidine deaminase turns on somatic hypermutation in hybridomas.
   Martin A, Bardwell P D, Woo C J, Fan M, Shulman M J, Scharff M D.
   Nature. 2002 Feb. 14; 415(6873):802-6. Epub 2002 Jan. 30.
9. AID mutates E. coli suggesting a DNA deamination mechanism for antibody diversification.
   Petersen-Mahrt S K, Harris R S, Neuberger M S.
   Nature. 2002 Jul. 4; 418(6893):99-103.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine study, numerous equivalents to the specific procedures described herein. Such equivalents are considered to be within the scope of this invention and are covered by the claims. All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference. The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps

The term “or combinations thereof” as used herein refers to all permutations and combinations of the listed items preceding the term. For example, “A, B, C, or combinations thereof is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, MB, BBC, AAABCCCC, CBBAAA, CABABB, and so forth. The skilled artisan will understand that typically there is no limit on the number of items or terms in any combination, unless otherwise apparent from the context.

Any part of this disclosure may be read in combination with any other part of the disclosure, unless otherwise apparent from the context.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

The present invention is described in more detail in the following non limiting Example.

### EXAMPLES

The following proposed protocol will be useful for replacing one or more exons or active-site loops in a base AID gene. For example, for replacing at least exon 3 in a mouse or rat AID gene (the base AID gene) with exon 3 nucleotide sequence from an AID gene of a different species, eg, chicken, Xenopus or human, or with an exon from an APOBEC member.

(a) Generation of BAC Clones Ready for Recombineering

Sequence manipulation can be carried out using standard recombineering techniques (Lee, E. et al. Genomics, 73: 56-65; Chan, W. et al. Nucleic Acids Research, 35, e64) and bacterial artificial chromosomes (BACs) according to the following proposed protocol. In order to make a BAC clone, BAC0001, ready for recombineering, overnight cultures containing the BAC (e.g., a 129 strain BAC clone obtainable from The Sanger Institute, Hinxton, UK) will be grown from single colonies, diluted 50-fold in LB medium, and grown to an OD₆₀₀=0.6-0.9. Ten-milliliter cultures will then be washed with ice-cold sterile water for three times. Cells are then resuspended in 50 μl of ice-cold sterile water and electroporated by pSIM18 (Chan, W. et al. Nucleic Acids Research, 35, e64) using Bio-Rad gene pulser set at 1.75 kV, 25 μF with a pulse controller set at 200 ohms. Cells will be incubated at 32° C. for 1.5 h with shaking and spread on agar media with 20 μg/ml of hygromycin.

(b) Exon Replacement

Overnight cultures containing the BAC and pSIM18 growing at 32° C. will be diluted 50-fold in LB medium, and grown to an OD₆₀₀0=0.6-0.9. Ten-milliliter cultures will then be induced for Red expression by shifting the cells to 42° C. for 15 min followed by chilling on ice for 20 min. Cells will then be washed with ice-cold sterile water for three times. Cells will then be resuspended in 50 μl of ice-cold water and electroporated under the conditions mentioned above with 100 ng of linear DNA containing a sacB-Neo cassette which is designed for use in the stepwise replacement of exon(s) of the base AID gene. A suitable sacB-Neo cassette is one derived from the pEL04 vector described in Lee, E. et al. Genomics, 73: 56-65 (the disclosure of which including details of vector design and construction, is incorporated herein by reference), but with cat^{R} replaced by neo^{R}. The correct modified BAC clones will then be selected on agar media with 25 μg/ml of kanamycin and confirmed by the corresponding junction. The sacB-Neo cassette targeted in the BAC will be further replaced with a corresponding exon from a gene encoding an orthologue or homologue AID or APOBEC by targeting a linear DMA with the exon flanked by homology arms and selection by agar media with 5% sucrose. Each exon will be replaced one by one. In one embodiment exon 3 is replaced, eg, exon 3 alone is replaced. In another example, exon 3 is replaced and then exon 2, optionally then exon 4, optionally then exon 5.

The design of suitable homology arms will be apparent to the skilled person having regard to regions of sequence upstream and downstream of the exon to be replaced, eg, nucleotide sequences immediately flanking said exon.

### (c) Generation of Cassettes for Exon Replacement

For all primers listed below, nucleotides in italics are homologous to the targeted sequence, while those in Roman type are homologous to the amplification cassette.

The sacB-Neo cassette that can be used to replace the exon 2 of the mouse AICDA gene in the BAC clone, BAC0001 can be amplified from a vector containing a sacB-Neo cassette with PRIMER 1 and PRIMER 2:

PRIMER 1:
5′ACAATAATAATCAGAGCTGAAGGAAGACTATGGTGACAGAGAAGCCTTGCCCTGACTTTCTTCTCCAACTCACAG
CTGTGACGGAAGATCACTTCG3′
PRIMER 2:
5′CACCAGGGGCAGCCATAGCTTTAGTGTCAACAGCTGCCACCCACCCCCTCCCCAACCCCGCAACCCCCCCCCCACC
TGAGGTTCTTATGGCTCTTG3′

The sacB-Neo cassette used to replace exon 3 of a mouse AID gene is amplified with PRIMER 3 and PRIMER 4:

PRIMER 3:
:5′CCCACAAGCATCCCAAATGGCCTGGGTGGGAGAGCATGCAGGTCACGTCACCAGTGCTCTCTGCTCTTTCTCCA
GCTGTGACGGAAGATCACTTCG3′
PRIMER 4:
5′CCCACCCCCAGTTTCCCCGCTGACACTCACTCTGAGTGGCAACTCAGACCGCTCTCTCCAGTGTGCAAGTCTCACC
TGAGGTTCTTATGGCTCTTG3′

The sacB-Neo cassette used to replace exon 4 of a mouse AID gene is amplified with PRIMER 5 and PRIMER 6:

PRIMER 5:
5′ACACACACACACACACACACACACACACACACACACACCTCCTTCTTATTTATCTATTTATTTTTCTTTTAACGTG
TGACGGAAGATCACTTCG3′
PRIMER 6:
5′GAGAGAGAGAGAGACAGAGACAGACAGAGAGACAGAGACAGACAGAGAGACAGGCAGACAGACAGGCAGAC
TTACCTGAGGTTCTTATGGCTCTTG3′

To replace the CDS (coding sequence) in the exon 5 of a mouse AID gene as well as to insert a selection marker that is useful in an ES cell targeting, a modifying vector is constructed by inserting 3′ untranslated region (AAGCAACCTCCTGGAATGTCACACGTGATGAAATTTCTCTGAAGAGACTGGATAGAAAAACAACCCTTCAACTAC ATGTTTTTCTTCTTAAGTACTCACTTTTATAAGTGTAGGGGGAAATTATATGACTTT) following a PiggyBac transposon, with a PGK-purodTK cassette at the NheI-MluI sites of the 3′ end of the sacB-Neo cassette. A suitable PGK-purodTK cassette is, for example, one derived from pPB-PGK-Neo (Wang, W, et al PNAS, 105, 9290-9295) by replacement of the Heo^{R} gene with the PurodTK gene. The sacB-Neo and PiggyBac transposon PGk-PurodTK cassette that will be used to replace the CDS in the exon 5 is amplified with PRIMER 7 and PRIMER 8:

PRIMER 7:
5′GTTTAGACACTTTCCTTTCCAGAGATCAAATTTAAAGCCCTTCACTCCGTTTATATCATCTCTCTTTCTCCACACGTG
TGACGGAAGATCACTTCG3′
PRIMER 8:
5′CCAGTAGATGGCGATGTTGCACAGCAAGCTCAGTTACATCATTGCTCTGGCGGTCCTGTGCAGCTCAAGTATTTT
CTGAGGTTCTTATGGCTCTTG3′

For targeting of exon 2, exon 3 or exon 4, the corresponding exon from an orthologue or homologue AID will be amplified from the relevant foreign (non-base species) gene (e.g., chicken, Xenopus or human AID gene), for example from genomic DNA, for use with the sacB-Neo cassette. Each such exon will be amplified from the foreign gene with a 5′ primer containing the same 5′ sequence used for homologous targeting (nucleotides in italics as shown above) plus the 3′ sequence homologous to the specific exons. For example, to replace the mouse AID exon 3 with Xenopus AID exon 3, the exon cassette is amplified from Xenopus genomic DNA with PRIMER 9 and PRIMER 10:

PRIMER 9:
5′CCCACAAGCATCCCAAATGGCCTGGGTGGGAGAGCATGCAGGTCACGTCACCAGTGCTCTCTGCTCTTTCTCCAG
AACGGCTGCCACGCTGAGATGCTCTTCCTGCG3′
PRIMER 10:
5′CCCACCCCCAGTTTCCCCGCTGACACTCACTCTGAGTGGCAACTCAGACCGCTCTCTCCAGTGTGCAAGTCTCACC
TTTGTAGCTCATGACAGACAGTC3′

The nucleotides in italic in both primers correspond to the 3′ of the intron 2 and 5′ of the intron 3 of mouse AID gene respectively, while the nucleotides in Roman correspond to the 5′ and 3′ of exon 3 of Xenopus AID gene respectively.

For the targeting of the CDS in the exon 5 from orthologues or homologues, the region is amplified from the foreign AID DNA with the 5′ primer with the same features as described as above (PRIMER 9), and the 3′ primer (PRIMER 11) as follows:

PRIMER 11:
5′AGGCAAAGCCTCCATCCAGACAGGCAGCCAGCACTACTGGAGCACATGCACAAGCAGATGAGACTGTCTTGTTA
C3′

with the sequence homologous to 5′ region of 3′UTR exon, plus the 3′ sequence homologous to the targeting CDS of the exon 5.

For example, to replace the CDS in exon 5 of mouse AID with the CDS in exon 5 of Xenopus AID, the region is amplified from Xenopus genomic DNA with PRIMER 12 and PRIMER 13:

PRIMER 12:
5′GTTTAGACACTTTCCTTTCCAGAGATCAAATTTAAAGCCCTTCACTCCGTTTATATCATCTCTCTTTCTCCACACGCG
CCGTACGACATGGAGG3′
PRIMER 13:
5′AGGCAAAGCCTCCATCCAGACAGGCAGCCAGCACTACTGGAGCACATGCACAAGCAGATGAGACTGTCTTGTTA
CTTAAAGCCCAAGTAGAACAAACACTTC3′.

For replacing the sequence encoding the active-site loop, first, the sacB-Neo cassette is amplified from the pEL05 vector by PRIMER 14 and PRIMER 15:

PRIMER14:
5′TATGACTGTGCCCGGCACGTGGCTGAGTTTCTGAGATGGAACCCTAACCTCAGCCTGAGGATTTTCACCGCGCGC
CTGTGACGGAAGATCACTTCG3′
PRIMER15:
5′CAGTGTGCAAGTCTCACCTTTGAAGGTCATGATCCCGATCTGGACCCCAGCGCGGTGCAGTCTCCGCAGCCCCTC
CTGAGGTTCTTATGGCTCTTG3′

Following the replacement of the active-site loop-encoding sequence in the mouse AID gene with the sacB-Neo cassette, the DNA fragment containing the sequence encoding the active-site loop from orthologues or homologues flanked by 5′ homology arm

### TATGACTGTGCCCGGCACGTGGCTGAGTTTCTGAGATGGAACCCTAACCTCAGCCTGAGGATTTTCACCGCGCGC; SEQ ID NO: 67) and

3′ homology arm (GAGGGGCTGCGGAGACTGCACCGCGCTGGGGTCCAGATCGGGATCATGACCTTCAAAG; SEQ ID NO: 68)

is amplified and targeted to replace the sacB-Neo cassette. For example, to replace the mouse AID active-site loop with a Xenopus AID one, the Xenopus one is amplified from Xenopus genomic DMA with PRIMER 16 and PRIMER 17:

PRIMER16:
5′TATGACTGTGCCCGGCACGTGGCTGAGTTTCTGAGATGGAACCCTAACCTCAGCCTGAGGATTTTCACCGCGCGC
CTCTATTTCTGCGAGGAGCG3′
PRIMER17:
5′CTTTGAAGGTCATGATCCCGATCTGGACCCCAGCGCGGTGCAGTCTCCGCAGCCCCTCCGGCTCCGCGTTGCGCT
CCT3′

### Nucleotide Sequence Encoding the Active-Site Loop

| | |
|---|---|
| Human | CTCTACTTCTGTGAGGACCGCAAGGCTGAGCCC |
| Mouse | CTCTACTTCTGTGAAGACCGCAAGGCTGAGCCT |
| Chicken | CTCTACTTCTGTGAAGATCGCAAGGCTGAGCCT |
| Xenopus | CTCTATTTCTGCGAGGAGCGCAACGCGGAGCCG |
| Catfish | CTCTACTTCTGTGACGAGGAGGACAGTCAAGAGAGA |
| Zebrafish | CTGTACTTCTGTGATGAAGAGGACAGCGTGGAGAGA |

### Amino Acid Sequence for the Active-Site Loop

| | |
|---|---|
| Human | LYFCEDRKAEP |
| Mouse | LYFCEDRKAEP |
| Chicken | LYFCEDRKAEP |
| Xenopus | LYFCEERNAEP |
| Catfish | LYFCDEEDSQER |
| Zebrafish | LYFCDEEDSVER |

Nucleotide sequence encoding the mouse AID mutant (Xenopus exon 3)—see SEQ ID NO: 53 Amino acid sequence for the mouse AID mutant (Xenopus exon 3)—see SEQ ID NO: 54 Nucleotide sequence encoding the mouse AID mutant (Xenopus active-site loop)—see SEQ ID NO:55 Amino acid sequence for the mouse AID mutant (Xenopus active-site loop)—see SEQ ID NO: 56 Nucleotide sequence encoding the mouse AID mutant (Catfish active-site loop)—see SEQ ID NO: 57 Amino acid sequence for the mouse AID mutant (Catfish active-site loop)—see SEQ ID NO: 58 Genomic Sequence of a Mouse AID—see SEQ ID NO: 23

(d) Generation of Targeting Vectors for Replacement of the AID Gene in ES Cells

The targeting vector to replace the mouse AID gene is generated by retrieving the genomic fragment from the modified BAC described above to the pBR322 vector. First, the 5′ retrieving arm (282 bp) will be amplified by PRIMER 18 and PRIMER 19 from the BAC clone, BAC0001, while the 3′ retrieving arm (313 bp) will be amplified by PRIMER 20 and PRIMER 21:

PRIMER 18:
5′AGGCGAATTCTCCATGAAAGTCAGGCTGGC3′,
PRIMER 19:
5′ GTTAGAATGACGATATCGGATCCATGCTAGTCTGGAAATCTC 3′
PRIMER 20:
5′TGGATCCGATATCGTCATTCTAACCACTGTTGTGCAC3′
PRIMER 21:
5′AGGCACGCGTCTAAACTGACTCCTCTTGTAGAC3′

PCR fragments will be purified, mixed and further amplified for bridge PCR by PRIMER 22 and PRIMER 23:

PRIMER22:
5′AGGCGAATTCTCCATGAAAGTCAGGCTGGC3′
PRIMER 23:
5′AGGCACGCGTCTAAACTGACTCCTCTTGTAGAC3′

The retrieving vector will be constructed by subcloning the amplified fragment (601 bp) into the EcoRI-MluI sites of the pBR322 vector amplified by PRIMER 24 and PRIMER 25:

PRIMER 24:
5′AGGCGAATTCTTTCTTAGACGTCAGGTGGCAC3′
PRIMER 25:
5′AGGCACGCGTCGATACGCGAGCGAACGTGA3′

Finally, the targeting vector will be generated by retrieving the 13 kb of modified genomic fragment into the EcoRV—linearised retrieving vector through conventional recombineering.

SEQUENCE CORRELATION TABLE

| SEQ ID NO: | Species | |
|---|---|---|
| | | cDNA |
| | | Access ID* |
| 1. | Homo sapiens | NM_020661.2 |
| | (Man) | |
| 2 | Pan troglodytes | NM_001071809.2 |
| | (Chimpanzee) | |
| 3 | Bas Taurus | NM_001038682.1 |
| | (Bovine) | |
| 4 | Canis lupus | NM_001003380.1 |
| | (Dog) | |
| 5 | Oryctolagus cuniculus | XM_002712854.1 |
| | (Rabbit) | |
| 6 | Rattus norvegicus | NM_001100779.1 |
| | (Rat) | |
| 7 | Mus musculus | NM_009645.2 |
| | (Mouse) | |
| 8 | Gallus gallus | XM_416483.1 |
| | (Chicken) | |
| 9 | Xenopus laevis | NM_001095712.1 |
| | (African clawed frog) | |
| 10 | Ictalurus punctatus | AY436507.1 |
| | (Channel Catfish) | |
| 11 | Danio rerio | NM_001008403.1 |
| | (Zebra fish) | |
| | | Protein |
| | | Access ID |
| 12 | Homo sapiens | NP_065712.1 |
| | (Man) | |
| 13 | Pan troglodytes | NP_001065277.1 |
| | (Chimpanzee) | |
| 14 | Bos Taurus | NP_001033771.1 |
| | (Bovine) | |
| 15 | Canis lupus | NP_001003380.1 |
| | (Dog) | |
| 16 | Oryctolagus cuniculus | XP_002712900.1 |
| | (Rabbit) | |
| 17 | Rattus norvegicus | NP_001094249.1 |
| | (Rat) | |
| 18 | Mus musculus | NP_033775.1 |
| | (Mouse) | |
| 19 | Gallus gallus | XP_416483.1 |
| | (Chicken) | |
| 20 | Xenopus laevis | NP_001089181.1 |
| | (African clawed frog) | |
| 21 | Ictalurus punctatus | AAR97544.1 |
| | (Channel Catfish) | |
| 22 | Danio rerio | NP_001008403.1 |
| | (Zebra fish) | |

*Access ID for nucleotide sequences is the ID for nucleic acid
(not necessarily cDNA sequences) that comprise a nucleotide sequence
encoding AID from the species indicated

| SEQ ID NO: | Description |
|---|---|
| 23 | Genomic Sequence of Mouse AID |
| 24 | PRIMER 1 |
| 25 | PRIMER 2 |
| 26 | PRIMER 3 |
| 27 | PRIMER 4 |
| 28 | PRIMER 5 |
| 29 | PRIMER 6 |
| 30 | PRIMER 7 |
| 31 | PRIMER 8 |
| 32 | PRIMER 9 |
| 33 | PRIMER 10 |
| 34 | PRIMER 11 |
| 35 | PRIMER 12 |
| 36 | PRIMER 13 |
| 37 | PRIMER 14 |
| 38 | PRIMER 15 |
| 39 | PRIMER 16 |
| 40 | PRIMER 17 |
| 41 | Nucleotide sequence encoding human AID |
| | active-site loop |
| 42 | Nucleotide sequence encoding mouse AID |
| | active-site loop |
| 43 | Nucleotide sequence encoding chicken AID |
| | active-site loop |
| 44 | Nucleotide sequence encoding Xenopus AID |
| | active-site loop |
| 45 | Nucleotide sequence encoding catfish AID |
| | active-site loop |
| 46 | Nucleotide sequence encoding zebrafish AID |
| | active-site loop |
| 47 | Amino acid sequence of human AID active- |
| | site loop |
| 48 | Amino acid sequence of mouse AID active- |
| | site loop |
| 49 | Amino acid sequence of chicken AID active- |
| | site loop |
| 50 | Amino acid sequence of Xenopus AID active- |
| | site loop |
| 51 | Amino acid sequence of catfish AID active-site |
| | loop |
| 52 | Amino acid sequence of zebrafish AID active- |
| | site loop |
| 53 | Nucleotide sequence encoding Chimaeric AID |
| | (mouse AID with Xenopus exon 3) |
| 54 | Amino acid sequence of Chimaeric AID |
| | (mouse AID with Xenopus exon 3) |
| 55 | Nucleotide sequence encoding Chimaeric AID |
| | (mouse AID with Xenopus active-site loop) |
| 56 | Amino acid sequence of Chimaeric AID |
| | (mouse AID with Xenopus active-site loop) |
| 57 | Nucleotide sequence encoding Chimaeric AID |
| | (mouse AID with catfish active-site loop) |
| 58 | Amino acid sequence of Chimaeric AID |
| | (mouse AID with catfish active-site loop) |
| 59 | PRIMER 18 |
| 60 | PRIMER 19 |
| 61 | PRIMER 20 |
| 62 | PRIMER 21 |
| 63 | PRIMER 22 |
| 64 | PRIMER 23 |
| 65 | PRIMER 24 |
| 66 | PRIMER 25 |
| 67 | 5′ homology arm |
| 68 | 3′ homology arm |

SEQUENCE LISTING

SED ID NO: 1
ATGGACAGCCTCTTGATGAACCGGAGGAAGTTTCTTTACCAATTCAAAAATGTCCGCTGGGCTAAGGGTCGGCGT
GAGACCTACCTGTGCTACGTAGTGAAGAGGCGTGACAGTGCTACATCCTTTTCACTGGACTTTGGTTATCTTCGCA
ATAAGAACGGCTGCCACGTGGAATTGCTCTTCCTCCGCTACATCTCGGACTGGGACCTAGACCCTGGCCGCTGCTA
CCGCGTCACCTGGTTCACCTCCTGGAGCCCCTGCTACGACTGTGCCCGACATGTGGCCGACTTTTCTGCGAGGGAAC
CCCAACCTCAGTCTGAGGATCTTCACCGCGCGCCTCTACTTCTGTGAGGACCGCAAGGCTGAGCCCGAGGGGCTG
CGGCGGCTGCACCGCGCCGGGGTGCAAATAGCCATCATGACCTTCAAAGATTATTTTTACTGCTGGAATACTTTTG
TAGAAAACCACGAAAGAACTTTCAAAGCCTGGGAAGGGCTGCATGAAAATTCAGTTCGTCTCTCCAGACAGCTTC
GGCGCATCCTTTTGCCCCTGTATGAGGTTGATGACTTACGAGACGCATTTCGTACTTTGGGACTTTGA
SEQ ID NO: 2
ATGGACAGCCTCTTGATGAACCGGAAGAAGTTTCTTTACCAATTCAAAAATGTCCGCTGGGCTAAGGGTCGGCGT
GAGACCTACCTGTGCTACGTAGTGAAGAGGCGGGACAGTGCTACATCCTTTTCACTGGACTTTGGTTATCTTCGCA
ATAAGAACGGCTGCCACGTGGAATTGCTCTTCCTCCGCTACATCTCGGACTGGGACCTAGACCCTGGCCGCTGCTA
CCGCGTCACCTGGTTCACCTCCTGGAGCCCCTGCTACGACTGTGCCCGACATGTGGCCGACTTTCTGCGAGGGAAC
CCCAACCTCAGTCTGAGGATCTTCACCGCGCGCCTCTACTTCTGTGAGGACCGCAAGGCTGAGCCCGAGGGGCTG
CGGCGGCTGCACCGCGCCGGGGTGCAAATAGCCATCATGACCTTCAAAGATTATTTTTACTGCTGGAATACTTTTG
TAGAAAACCATGAAAGGACTTTCAAAGCCTGGGAAGGGCTGCATGAAAATTCAGTTCGTCTCTCCAGACAGCTTC
GGCGCATCCTTTTGCCCCTGTATGAGGTTGATGACTTACGAGACGCATTTCGTACTTTGGGACTTTGA
SEQ ID NO: 3
ATGGACAGCCTCTTGAAGAAGCAGAGACAGTTTCTTTACCAGTTCAAAAACGTGCGCTGGGCTAAGGGCCGCCAT
GAGACCTACTTGTGCTACGTGGTGAAGCGGCGGGACAGTCCCACCTCCTTCTCACTGGACTTCGGGCACCTTCGAA
ACAAGGCCGGATGCCACGTGGAGTTGCTCTTCCTTCGCTACATCTCTGACTGGGATCTGGACCCTGGGCGGTGCTA
CCGCGTCACCTGGTTCACGTCTTGGAGCCCCTGCTACGACTGTGCGCGGCACGTGGCCGACTTCCTGCGGGGGTA
CCCCAACCTGAGCCTGCGGATCTTCACGGCGCGCCTCTACTTCTGCGACAAGGAGCGCAAGGCCGAGCCAGAGGG
GCTGCGGCGGCTGCACCGCGCTGGAGTCCAGATCGCCATCATGACGTTCAAAGATTATTTTTATTGCTGGAATACT
TTTGTGGAAAATCATGAAAGAACTTTCAAAGCCTGGGAGGGACTGCATGAAAATTCGGTTCGTCTGTCTAGACAG
CTTCGACGCATCCTTTTGCCACTCTACGAGGTTGATGACTTGCGGGATGCATTTCGTACTTTGGGACTTTGA
SEQ ID NO: 4
ATGGACAGCCTCCTGATGAAGCAGAGGAAGTTTCTTTACCATTTCAAGAATGTCCGCTGGGCGAAGGGTCGCCAT
GAGACTTACTTGTGCTACGTGGTGAAGCGGCGGGATAGTGCCACCTCCTTTTCTCTGGACTTTGGTCACCTTCGAA
ACAAGTCGGGCTGCCACGTGGAGCTGCTCTTCCTCCGCTACATCTCCGACTGGGACCTGGACCCCGGCCGGTGCTA
CCGCGTCACCTGGTTCACGTCCTGGAGCCCCTGCTACGACTGCGCGCGGCACGTGGCGGACTTCCTGCGCGGGTA
CCCCAACCTCAGCCTCAGGATCTTCGCCGCGCGCCTCTACTTCTGCGAGGACCGCAAGGCGGAGCCCGAGGGGCT
GCGGCGGCTGCACCGGGCGGGCGTCCAGATCGCCATCATGACCTTCAAGGATTATTTTTATTGCTGGAATACTTTT
GTGGAAAATCGTGAAAAAACTTTCAAAGCCTGGGAGGGGTTGCACGAAAATTCCGTTCGACTATCCAGACAGCTT
CGACGCATTCTTTTGCCCCTGTATGAGGTTGATGACTTACGAGATGCATTTCGTACTTTGGGACTTTGA
SEQ ID NO: 5
ATGCCGCAGACCCGCTCCTCGCCGCTGGTCCTCCTTTTGATGAAGCAGAAGAAGTTTCTTTATCACTTCAAGAATGT
CCGCTGGGCTAAGGGCCGGCACGAGACCTACCTGTGCTACGTGGTCAAGCGGCGGGACAGTGCCACCTCCTTCTC
ACTGGACTTCGGCTACCTGCGCAACACGAACGGCTGCCACGTGGAATTGCTCTTCCTCCGCTACATCTCCGACTGG
GACCTGGACCCCGGCCGCTGCTACCGCGTCACCTGGTTCACCTCCTGGAGCCCTTGCTACGACTGTGCCCGGCACG
TGGCTGACTTCCTGAGAGGCAACCCCAACCTCACTCTGAGGATCTTCACCGCGCGCCTCTACTTCTGCGAGGACCG
CAAGGCCGAGCCCGAGGGACTGCGGCGGCTGCACCAAGCGGGCGTCCAGCTCGGCATCATGACCTTCAAAGATT
ATTTTTACTGCTGGAATACTTTCGTGGAGAACCGTGAGAGAACGTTCAAGGCCTGGGAAGGCCTGCATGAAAATT
CTGTCCGCCTGTCCAGACAGCTCCGGCGCATCCTTCTGCCCCTTTATGAGGTCGATGACCTACGAGATGCGTTTCGT
ACTTTGGGACTTTGA
SEQ ID NO: 6
ATGGACAGCCTCTTGATGAAGCAAAAGAAGTTTCTTTACCACTTCAAAAATGTCCGCTGGGCTAAGGGTCGGCAC
GAGACCTACCTGTGCTATGTGGTGAAGAGGAGAGATAGTGCCACCTCCTTCTCACTGGACTTTGGCCACCTTCGCA
ACAAGTCGGGCTGCCACGTGGAATTGTTGTTCCTACGCTACATCTCGGACTGGGACCTGGACCCCGGCCGGTGTTA
CCGTGTCACCTGGTTCACTTCCTGGAGCCCCTGCTACGACTGTGCGCGGCACGTGGCTGAGTTTCTGAGATGGAAC
CCTAACCTCAGCCTGAGGATTTTCACCGCGCGCCTCTACTTCTGCGAAGACCGCAAGGCTGAGCCTGAGGGGCTGC
GGAGGCTGCACCGCGCCGGAGTCCAGATCGGGATCATGACCTTCAAAGACTATTTTTACTGCTGGAATACATTTGT
AGAAAATCATGAAAGAACTTTCAAAGCCTGGGAAGGGCTGCATGAAAACTCCGTCAGGCTAACCAGACAGCTTCG
GCGCATCCTTTTGCCCTTGTATGAAGTCGATGACTTGAGAGATGCGTTTCGTATTTTGGGACTTTGA
SEQ ID NO: 7
ATGGACAGCCTTCTGATGAAGCAAAAGAAGTTTCTTTACCATTTCAAAAATGTCCGCTGGGCCAAGGGACGGCAT
GAGACCTACCTCTGCTACGTGGTGAAGAGGAGAGATAGTGCCACCTCCTGCTCACTGGACTTCGGCCACCTTCGCA
ACAAGTCTGGCTGCCACGTGGAATTGTTGTTCCTACGCTACATCTCAGACTGGGACCTGGACCCGGGCCGGTGTTA
CCGCGTCACCTGGTTCACCTCCTGGAGCCCGTGCTATGACTGTGCCCGGCACGTGGCTGAGTTTCTGAGATGGAAC
CCTAACCTCAGCCTGAGGATTTTCACCGCGCGCCTCTACTTCTGTGAAGACCGCAAGGCTGAGCCTGAGGGGCTGC
GGAGACTGCACCGCGCTGGGGTCCAGATCGGGATCATGACCTTCAAAGACTATTTTTACTGCTGGAATACATTTGT
AGAAAATCGTGAAAGAACTTTCAAAGCCTGGGAAGGGCTACATGAAAATTCTGTCCGGCTAACCAGACAACTTCG
GCGCATCCTTTTGCCCTTGTACGAAGTCGATGACTTGCGAGATGCATTTCGTATGTTGGGATTTTGA
SEQ ID NO: 8
ATGGACAGCCTCTTGATGAAGAGGAAGCTCTTCCTCTACAATTTCAAGAACCTGCGCTGGGCCAAAGGCCGTCGT
GAAACCTACCTCTGTTATGTTGTGAAGCGCCGTGACAGTGCTACATCATGCTCCCTGGACTTTGGATACCTGCGTA
ACAAGATGGGTTGCCATGTGGAGGTTCTCTTCCTACGCTACATCTCAGCTTGGGACCTGGACCCAGGCCGCTGCTA
CCGCATCACATGGTTCACCTCCTGGAGCCCCTGTTATGACTGTGCCCGACATGTGGCTGACTTCCTTCGTGCCTACC
CAAACTTGACCCTCCGCATTTTCACTGCCCGCCTCTACTTCTGTGAAGATCGCAAGGCTGAGCCTGAGGGGCTGAG
ACGCCTGCACCGGGCTGGGGCCCAAATCGCCATCATGACTTTCAAAGATTTCTTCTACTGCTGGAACACGTTTGTG
GAGAACAGGGAAAAGACATTCAAAGCCTGGGAAGGGCTGCATGAAAACTCTGTCCATCTGTCCAGGAAACTCCG
ACGGATCCTTCTGCCACTGTATGAAGTAGATGATTTACGAGATGCCTTTAAAACTCTGGGACTTTGA
SEQ ID NO: 9
ATGACGATGGACAGCATGTTGTTGAAGCGCAACAAGTTCATCTATCACTACAAGAACCTGCGCTGGGCCCGGGGT
CGGCACGAGACCTACCTGTGCTACATAGTCAAGCGGAGATACAGCTCAGTGTCCTGCGCGTTGGACTTCGGGTAC
CTGCGGAACCGCAACGGCTGCCACGCTGAGATGCTCTTCCTGCGCTACCTGTCTATATGGGTGGGTCACGACCCCC
ATAGGAACTACCGGGTCACGTGGTTCAGCTCCTGGAGCCCCTGCTATGACTGTGCCAAGCGCACCCTCGAGTTCTT
AAAGGGGCACCCCAACTTCAGTCTGCGCATCTTCAGCGCCAGGCTCTATTTCTGCGAGGAGCGCAACGCGGAGCC
GGAGGGGCTGCGGAAACTGCAGAAAGCGGGGGTGCGACTGTCTGTCATGAGCTACAAAGATTATTTCTACTGCT
GGAACACCTTTGTGGAGACCCGGGAGAGCGGCTTTGAAGCCTGGGATGGATTACACGAGAACTCGGTCAGACTG
GCCCGGAAGCTGCGGCGCATCTTGCAGCCGCCGTACGACATGGAGGATCTGAGAGAAGTGTTTGTTCTACTTGGG
CTTTAA
SEQ ID NO: 10
ATGAGCAAGCTGGACAGTGTGCTGCTGACTCAGAGGAAGTTTATTTACCACTATAAGAATGTGCGCTGGGCTCGT
GGGAGGAACGAGACCTACCTCTGTTTTGTGGTCAAGAAACGCAACAGTCCCGACTCGCTCTCCTTCGACTTCGGAC
ACCTGCGCAATCGTTCTGGCTGCCATGTGGAGCTTCTCTTCCTGAGCTATCTTGGGGTACTGTGCCCAGGTTTCTTG
GGTTCCGGTGTGGATGGTGTCAGGGTGGCTTATGCCATCACCTGGTTCTGTTCCTGGTCACCCTGTTCAAACTGTG
CCCATCGCCTTTCTCGCTTCATGTCTCAGATGCCCAACCTGCGGCTGCGCATCTTCGTCTCGCGCCTCTACTTCTGTG
ACGAGGAGGACAGTCAAGAGAGAGAGGGACTCCGTTGCTTGCAGAGGGCAGGTGTGCAAGTGACAGTCATGAC
CTATAAAGATTTTTTCTACTGTTGGCAAACCTTTGTGGCTCAAAATCAGAAGGCTTTCAAGGCTTGGGACGACCTTC
ACCAGAACTCTATCCGACTGTCTCGGAAACTACAGCGAATCCTGCAGCCTAGTGAGTCTGAAGACCTGAGGGATG
GCTTCGCTCTGCTGGGCCTTTAA
SEQ ID NO: 11
ATGATCTGCAAGCTGGACAGTGTGCTCATGACCCAGAAGAAATTCATCTTCCACTATAAGAATGTGCGCTGGGCTC
GAGGGAGACACGAAACCTACCTTTGTTTTGTAGTAAAGCGACGCATCGGCCCTGATTCCCTCTCTTTTGACTTTGGA
CACCTGCGCAATCGCTCCGGATGCCATGTAGAGCTTCTCTTTCTGCGTCACTTGGGTGCGTTGTGTCCGGGCCTGA
GCGCTTCCAGTGTGGACGGTGCAAGATTGTGTTACTCAGTGACCTGGTTCTGCTCCTGGTCTCCCTGCTCTAAATGC
GCTCAACAGCTCGCCCACTTCCTGTCACAGACGCCCAATCTGAGGCTGAGGATCTTTGTGTCACGCCTGTACTTCTG
TGATGAAGAGGACAGCGTGGAGAGAGAAGGTCTGCGACACCTGAAGAGGGCAGGAGTTCAGATCTCGGTCATG
ACTTATAAAGACTTTTTCTACTGCTGGCAAACGTTTGTTGCAAGGAGGGAGCGGAGTTTTAAAGCCTGGGATGGA
CTTCATGAAAACTCTGTCCGGCTTGTTCGAAAACTCAATCGGATTCTGCAGCCTTGCGAAACTGAGGATCTGAGGG
ATGTTTTTGCTCTTCTTGGGTTATGA
SEQ ID NO: 12
MDSLLMNRRKFLYQFKNVRWAKGRRETYLCYVVKRRDSATSFSLDFGYLRNKNGCHVELLFLRYISDWDLDPGRCYRV
TWFTSWSPCYDCARHVADFLRGNPNLSLRIFTARLYFCEDRKAEPEGLRRLHRAGVQIAIMTFKDYFYCWNTFVENHER
TFKAWEGLHENSVRLSRQLRRILLPLYEVDDLRDAFRTLGL
SEQ ID NO: 13
MDSLLMNRKKFLYQFKNVRWAKGRRETYLCYVVKRRDSATSFSLDFGYLRNKNGCHVELLFLRYISDWDLDPGRCYRV
TWFTSWSPCYDCARHVADFLRGNRNLSLRIFTARLYFCEDRKAEPEGLRRLHRAGVQIAIMTFKDYFYCWNTFVENHER
TFKAWEGLHENSVRLSRQLRRILLPLYEVDDLRDAFRTLGL
SEQ ID NO: 14
MDSLLKKQRQFLYQFKNVRWAKGRHETYLCYVVKRRDSPTSFSLDFGHLRNKAGCHVELLFLRYISDWDLDPGRCYRV
TWFTSWSPCYDCARHVADFLRGYPNLSLRIFTARLYFCDKERKAEPEGLRRLHRAGVQIAIMTFKDYFYCWNTFVENHE
RTFKAWEGLHENSVRLSRQLRRILLPLYEVDDLRDAFRTLGL
SEQ ID NO: 15
MDSLLMKQRKFLYHFKNVRWAKGRHETYLCYVVKRRDSATSFSLDFGHLRNKSGCHVELLFLRYISDWDLDPGRCYRV
TWFTSWSPCYDCARHVADFLRGYPNLSLRIFAARLYFCEDRKAEPEGLRRLHRAGVQIAIMTFKDYFYCWNTFVENREK
TFKAWEGLHENSVRLSRQLRRILLPLYEVDDLRDAFRTLGL
SEQ ID NO: 16
MPQTRSSPLVLLLMKQKKFLYHFKNVIRWAKGRHETYLCYVVKRRDSATSFSLDFGYLRNTNGCHVELLFLRYISDWDLD
PGRCYRVTWFTSWSPCYDCARHVADFLRGNPNLTLRIFTARLYFCEDRKAEPEGLRRLHQAGVQLGIMTFKDYFYCWN
TFVENRERTFKAWEGLHENSVRLSRQLRRILLPLYEVDDRDAFRTLGL
SEQ ID NO: 17
MDSLLMKQKKFLYHFKNVRWAKGRHETYLCYVVKRRDSATSFSLDFGHLRNKSGCHVELLFLRYISDWDLDPGRCYRV
TWFTSWSPCYDCARHVAEFLRWNPNLSLRIFTARLYFCEDRKAEPEGLRRLHRAGVQIGIMTFKDYFYCWNTFVENHE
RTFKAWEGLHENSVRLTRQLRRILLPLYEVDDLRDAFRILGL
SEQ ID NO: 18
MDSLLMKQKKFLYHFKNVRWAKGRHETYLCYVVKRRDSATSCSLDFGHLRNKSGCHVELLFLRYISDWDLDPGRCYRV
TWFTSWSPCYDCARHVAEFLRWNPNLSLRIFTARLYFCEDRKAEPEGLRRLHRAGVQIGIMTFKDYFYCWNTFVENRE
RTFKAWEGLHENSVRLTRQLRRILLPLYEVDDLRDAFRMLGF
SEQ ID NO: 19
MDSLLMKRKLFLYNFKNLRWAKGRRETYLCYVVKRRDSATSCSLDFGYLRNKMGCHVEVLFLRYISAWDLDPGRCYRIT
WFTSWSPCYDCARHVADFLRAYPNLTLRIFTARLYFCEDRKAEPEGLRRLHRAGAQIAIMTFKDFFYCWNTFVENREKT
FKAWEGLHENSVHLSRKLRRILLPLYEVDDLRDAFKTLGL
SEQ ID NO: 20
MTMDSMLLKRNKFIYHYKNLRWARGRHETYLCYIVKRRYSSVSCALDFGYLRNRNGCHAEMLFLRYLSIWVGHDPHR
NYRVTWFSSWSPCYDCAKRTLEFLKGHPNFSLRIFSARLYFCEERNAEPEGLRKLQKAGVRLSVMSYKDYFYCWNTFVE
TRESGFEAWDGLHENSVRLARKLRRILQPPYDMEDLREVFVLLGL
SEQ ID NO: 21
MSKLDSVLLTQRKFIYHYKNVRWARGRNETYLCFVVKKRNSPDSLSFDFGHLRNRSGCHVELLFLSYLGVLCPGFLGSGV
DGVRVAYAITWFCSWSPCSNCAHRLSRFMSQMPNLRLRIFVSRLYFCDEEDSQEREGLRCLQRAGVQVTVMTYKDFFY
CWQTFVAQNQKAFKAWDDLHQNSIRLSRKLQRILQPSESEDLRDGFALLGL
SEQ ID NO: 22
MICKLDSVLMTQKKFIFHYKNVRWARGRHETYLCFVVKRRIGPDSLSFDFGHLRNRSGCHVELLFLRHLGALCPGLSASS
VDGARLCYSVTWFCSWSPCSKCAQQLAHFLSQTPNLRLRIFVSRLYFCDEEDSVEREGLRHLKRAGVQISVMTYKDFFYC
WQTFVARRERSFKAWDGLHENSVRLVRKLNRILQPCETEDLRDVFALLGL
SEQ ID NO: 23
Genomic Sequence of Mouse AID

| | |
|---|---|
| 1. | The nucleotides in exons are labelled in upper case, and everything else in lower case. |
| 2. | The coding sequences are labelled in upper case with underlining, and the 5′UTR and 3′UTR in |
| | exons just in upper case. |
| 3. | The mouse AID gene covers 5 exons. |
| 4. | The 5 exons and 4 introns cover 10372 bp of DNA. |

5′-
caagagctagggacgcatccaaaagaaccggcaaccgggcccaacagacgttcattttcctgtttgttacatcatccacggtaagcaaggacagcga
cagctcaagtcttcaccagaagatgaaggtatcaacaaaacacagtaagggatggaagtctgatacgtggtctaatgtgggcagcttttgaagacgtt
gggcaaaagtagcccgcagcacagcaagcgcaaggccaatttgtactacatatggaatctttcttgaaaccaaacaaagaacaatcaagaaaaagg
aagaaaggatggaagggagggagggagggaaagagacaggaagcaaacacatcgggacaggcgactttggcttccagttatcttgaacaggcta
attcacagaaacagaaggtaggttagaggtcgctagggtctggggagtaagggacagctagtgactgttatgtaggtgtatttatttactgattgattg
actgatgcaatgaaagttttagggtagggtctggagagatggctcagtgtctaagagcacatattctgtagaggactctggttcagttcccagcaccca
caccaggcagcccacaactgcctgtaccaccagagaacataacacaccagtcctccaggcacacacacacacacacacacacacacacacacaca
cacacacacacacacacgcgcgcgcgcgcacacacatgcatgcacgcacgcacacacacacacacattctttaaggtttttttgtttgttttggttttttt
gttgtctttctttttgctttatgttttgtttgtttgtttatttgtttgtttttgagaccggctctatgtcctggaattttccatgtagacgaggctggcttg
aactcacagagatctgcctacctatgcctcctgaatggtagaattaaaggtgccactacatttcgctctaaaattaaaatttaaaaataaaagttttagggt
gggtgagatggttctggaagtaaaggcatttgccaccatcctggaaccccggtgatggaaggcaaaaacggacttctgaaatttgtcctgacctccac
acacacactaaataaatataaaatttacaaattggtttaaattttagaaacaaacagacctgctacgcaagcatgcattctgagtactcagaaggcag
aggcaagaggagccggaactcagccccctgacttgtcctgccccacaaaaggatgagaaaggtttaggttccgagtgtaaccattgccacagaatc
ctgcacttaagcaaagaaacaagcaagcaaacaaacaaacagaaacgccacagacaaacagaagataagcatcaacaatacgctgcttttctccg
gtccaaaaggccccagtttgcctagagagaccacgcagagcctgcgcagccacattcagagcaagccgcagtggtgtggaacctctccttgaagacg
agaaaacatttcctttctttatttctatgttttgttttttgtttttgttttttagcagggttccatgattgtcctggaactggacacatagcccaggctagt
ctcaaacttccaggaatcctcctgccttaatcttcagaatgctagaattctgatcgtgtacgactgccatacttgtcttgggggcgggattgcctgttccgc
ttgctgtctggcgacagggtttcactatgtagcccttggttggtctggaatacctttccttctttcttccttaaacatttgaaagatttatttattttatgt
atgtgagtacaccgtagttgtcttcagaagcaccagaagagggcatcagatcacaataaagatggttgtgagccaccatgtggttgctgggaattgaacgca
ggacctctggaagagcagtcagtgctcttaaccactgagccatctctccagctcgcccactggcttccttgcttgctttttcttaagttttatttatttatt
tatttatttatttatttatttagttatttagttatttatttagttatgtatattggtattttttcgaagaggacatcagatttcatcttagacggttgtgag
ccaccatgtgaatgcagagttgaacccaggtcctctgaaagaacagccagtgctctaaatactgaaccatctctccagcccctgctgctccctgtccctctc
ccttttaaagaaatggtgtcagtcagaacaggcaagatggttccatggataaatgtccttgctgcaaagcctgactacttgagttcaagcctcaggatc
tacatggtggacagagaggaccaagtcttgtaaattgtcttctgacatctacacataagctctggccctcgtgcctcatatacccctccactgccaagca
cagcaatatatatataattttttaaaatgtaaagaaatcacaacatctctgccaatatccatcaagtcggccctttgggaggctgtgtacgtgtgtctca
gtatgtcattccctggacaattggccaaagtagggcaaaggtccgggcctcatcctgtgagacaagttagagggacttgtccacccaccacctgggttc
ccttaaccctgtaatgtcacggctggtgctggttactcccggtgccctgaaatttttttcccaggaattcattaattcactagtgagggaaattgtgtctct
gatagtgatgtgataatgcagaggaaattaattagaggaagaaggaggatgggggctcattaacatttcagatatgatatccagggaaggctaaact
gccagggagtaagccaagtcctgaactatgagactttgcacagagagatttcacagcaacaaaataggggcaggggcatgtgctgtgtgcatgcaac
gggatccagtctctagctcaagactggtctggtctatatagaaagttccagaccagccagaggagctacataatgacaccctatctaaaaaaaggaa
gggaaggaaggaaggaaggaaggaaggaagaaaggaaggaaggagggagggagggagggagggagggagggagggaggaaagaaggaag
gaaggaaggaaggaaggaaggaaggaaggaaggaaggaaggaaggaaggaagagtataagaaaggaaggaaggaagaaagcaaaggatgtt
cttccagatgatcagggttcagatcccagcaaccacacatggtggctttcaaccgcctgctggtcctctgcaatagaaataagtgctcttaatcactggg
ccagctctccaggcctccagtaaggtatttttaatgaggaaaaagagttcttttttaaaaaaaaaatactttttgacacacacacacacacaaaattaa
aataaatcactttttggtgcaagcaactagtctttctagctatcttataatgtcattttaaaaaaagaaaaatatattagagaattaggaggctaaagtt
cactctctggatgctgtggtggtcaacccccatctctactgaggcataaaactgagtgtaacaaacggaaggaacagatactgtaagttcaagaagca
caagatgcatttaaggccactttaagtcactatgactgctatcattcttgttatcacaattttaaaattaggaagcatgcacagaccttaggtgtgatacc
tgggacccccccacacacacacacacacacactcacagagctcattatcatgataccaatgtgaaaagtgtccagtgctattgtctcctgatctttgtta
cctgtggtacctgggctggctttttagaggaacagcctcgaaggaagttggacattaagcatgagcagaactgccccccgccccccaatcatttaatcc
gtgtggctctgcccaccacagccccgcccatctttactggacccaacccaggaggcagatgttggatacctggtggtagtgatgctgtcgtgggggag
gagcccacaagagcaagctcagatttgaatgccaggggccagtgctctGTCACACAACAGCACTGAAGCAGCCTTGCTTGAAGCAA
GCTTCCTTTGGCCTAAGACTTTGAGGGAGTCAAGAAAGTCACGCTGGAGACCGATATGGACAGgtaacaagacagtct
catagcttgtgcatgtgctccagtagtgctggctgccgtctggatggaggctttgcctgtcagtgcgcgaatttcctcgtctgcttgccaccctctgctc
aggtcttttgggttttggacctaactctgaccacgaagttcttcccttcccccggtttctctcttctctgtgttgctagagataggaagccttgacttgtcc
tgagatttgggcagagctagagccggcttgtggtaataacagcgaagccttagaggcccgcgccacaaagaggtcgtagcaactccttactaaaaaca
gtagtggttattttcacaattatttggcaaatatccaacatcttaagactcgcatggggagtctttacaggaattatttagttatagcaagaagatttgta
cttctcaaaaaaaaaaaaaaaaaaaaaaaaactaaacatttgagatgaattgcttgcaactcattacaatggtgtctattgaaggagagaatttcatt
aagacaggcaatttagtgttatagactcaactgttagacacttggtgacatttttactgtttaattcatctatgcagagatttcttagcttcttgaaagctt
ttatatgcagctcatgatgagccattatcagaaatttctctcttgatttttacatttattgccagtgtgtgagtcactatgcctaaagcccatacacttgag
ctcacttccgtttggctatgaggtttagaatatggagttaatatagctaatggtagcagggtgttcttcagattccagatttttcctttcttgtcttccttc
tttctttttgttacccttctcctaccccctcttcttctccccctcctcctcttcctccctcctccccatctcttcccctcctcttctccctcctccccctcct
cttccccttcccctcttccccctcttcctcctcttcctcctccttctcctgctccccatctctttccctcctcttctccctcctcctcctccttttgcccctt
ctcctcttccccctcctcctcttccttttccttctcctccttctcctcctccctctcctccccctttcctcctcccactctccctcacccctatcagggacca
cattgaagacctcacacatgctagacaagtaatctgccacttaattacatcctgagccctcaaaaagcaaacagacagacagacagacagacaaacaaacaaa
caaacaaacaaatgttcacaggaggcaggcagacagcatgagctgcttctgggtttatagtgaattttgaaaccaaatctgagatctatgtcctgatggagaa
gggtccgagagaaatgcatgagcatggcaaaatgcaaagcaaagacgaggctgagattcagggagaagcaaacaagacagtggagagacacaggatg
gcacggcatggactggagcaagggcagcgggtaactcaaggcagccctgctactaggctgggattatttttaacccttgagtctggtttgcattgctg
gggaagcagctaaggttctgcctcaaggagcacagctgtctcagcagctggcgatctacaggtttgggacaccacctagcaaagtcctcataccggg
agggacatcccgaggagagggagctggaaataggctcctagctagagttgaggggagtgctggatggaggtgcccagtccacaggtcaggactgtg
cagacctcccaccgtggctggaatcttaaaatagaaacagtctattacatcttcctgtggttcagacacaactcttctatttgagacacatcctttctaa
actccaaggatacctttccttcataatttcagcatccacccccaatacacactcataaatacacaaacacacacacagagtaagagagagagaaaga
gagagataagcacgtatgtacacttgctacccacagtatgtaggaaaagttctctagggctgtgtgtacggctctgtggcacagcactcactagcaggt
acaagactccatgttcaacccactgaaaaagattctctacttttcccatctaggtaacacaggaagtttagttaaatagaaagggaatttattgctaag
agatgaagtttaagctgtttaaaactggctggattagagagatacctgtgcttattattataacatgctgagtttacctgtactgtggtggtgatgatgat
aatgatgctgtgtcatcacatagcccccgtggcttagaattctccatgaaagtcaggctggcttccattacagaaagatccacctgcctctgcccccctt
cgcccccaagttctggatttaaaggtgtgcacaccatgcccagcttctaaagggtttttataatttagtgatgaatgtagacatggaggtactatgatcg
ttatcatggtaaattactatttcaaaataaagctatgatcattagaggccaagacaggaggaccatgagttcgaggccagctgcagcaacatagagat
ttccagactagcatggatcccgcagcatgagcatgtccccaaaacaattttgtttttccaaaagtcagggactgtcacgtgtgttgaactatcattaaag
catgagctgtgaacgtgtgaacatgcattcaatgatagtatatggttatttatagtggctctaaccactgcagcaccaaagcggaacatatccaaattt
caatcagcacataaatgaataaacaaaacatgttctacccatacaatagaatattgctcggcaggaataaggagccaacttctgatatttgggtgaat
ataaaatttactatgttccgtgagagcagttacacaggaaggaggaaacgtgatttatatgaaattatagaaagttagaaataatttacatttacaga
gagcaggtcggcggttgcctgggtaagaggagaaagaacagccaatagcgacatagaagctttaagaagcctagaaatgtacctctgatggccctg
gcagtctgggctgcggacctgccggcattcacggagctgtagattttaagcgagtggagctcactatgtaaattgtatctcaacaacaacaaaagtga
aaaacggtttcaattctcttgcatcaaaaccgtattcaaattcctaactagctcttaaaaaaaaaatcattgcacttccatccatcaccactgtgtggcg
gtgctgtgtcgacaagtgagcgacacagttgtttatcatccgttttatctcctggctcatgtccaccgctttaacaggaactgtaatttttttttttttttaa
agaacgtgagggctgggaatatggttctgtgggaacagcgcttgccatgaaagcaaaaggacctgagttgaggggtccaaaagcagacacctgtaatt
cccgtgcttctaaggcaacataagaggtggagaaaggagaaccccaggaagcttatgagccagttagcccagggcgcacagcagagagcaagaga
ttctatctcaaacaagacagaagtcaaggaccaacacccaaggttgtactctgctcgccacacgtatcctgtagtatgtgttgcctctacccccaccac
atacacacacacgcacacactccacaaagattttaaaaattatttttaagtgtgtgtgtgtgtgtgtgtgtgtgtgtgtgtgtgtgtgtgtcaatgcatg
tgccctcaaaagtcagaggtgtcggatcctggtggaactggagtgacaggtggttgtgagctgcctgatggaagagcagttcgtgctcataactgctg
agccatactctagtccccagaaaacctgtatttttaaagaaagaaagaacgaacgagaaaaaaaaaacctcgggggctggagatgtagctctacta
gagaacttgccagcatgcacaaagccctgggttaagtccccaacaagtaggtgggacaagcctgttgtcccaagaccaggggagtagaggaggcag
gagagttctccttgtcaaattccccaccagcctgagctaaatgagttctcatctcaaaacaaacaataaaaataaataaataaaataaaatcaacaaa
actgacccagcaaaatccaaaaatgaaaacccaaagacctaagcaggggtgggggttaggggggctggtttgtcagacggctcagcaggtaaag
gcataaactgccaaacctgatgtcttgagtttgatccctcgagcacatgatggaagcagagagccaacttccacaagttgccctttgacctccacag
gtatgtgtatgtccctacacacatatcattcatacaacgatagtaaacaaatgtgatattttttttaaagacacagaggcaactatttcttatacttagttta
atgaagaatggatagatactatgtagcaacgtgatcgcaagtcgacatgacctcatatgaccctgtgcttagagagggaggaaggacgcccccagca
aggcccatctgcaacattccttttcctggatagagacaggacacccacagaatatggctcttcaaggaagagagtgacctttcttttcgcaggagctca
gtggctttgataccctgttgtcttccttcctcgctgtggctcaagtgctggaagtagagagtgactttctatgttttcctttgctttgtcttgtactgagtca
gacctagagcctcatacatgataggcaactgctgagctactgagctacgttcttgtggtggagctcaggctgggctcaaacttacagcaacctccctccc
ccagccttccccccactcccccccgccacccccccaccccccacccccgcactcccaagtctgaggttacaggcacaagcaacctaacctggcccttg
ccatgttttataacttgcttttggaagacttctggttctgtgatgctactgggttagcggggagacaggagggcagaaggttaaaggtgtctaagaccat
gtccaaagcccagtaggaggattagggagatggctgggctggagagatggctcagtggttaagagcactggctgtgtttgcaggggaccagagttca
attcccagcaaccacataatggctcacaatcatctacaatgggatctgacaccctcctttgacatgcaggcatgcatgtacacagagcagtcatacata
aattatataaataaatacattaaaaaataaaaagtaataaagggtaattacctagtttgactgttgcagcgaggggggaggggaagaggaaaggga
agagggtgggcagggaaggattttaaagtgagcatgtctcaggtatccagaaaaggaagcacgactatgctttctggtttaacctatataatgataag
atttaaaacatcatgatgatcaaagtaggcctggggatgcagctcggtgctgaagcgctcagctaccttgcctatggctgtaggtccagccatcagcag
ctgcaacaataataatcagagagaaggaagactatggtgacagagaagccttgccctgactttcttctccaactcacagCCTTCTGATGAAGC
AAAAGAAGTTTCTTTACCATTTCAAAAATGTCCGCTGGGCCAAGGGACGGCATGAGACCTACCTCTGCTACGTGGT
GAAGAGGAGAGATAGTGCCACCTCCTGCTCACTGGACTTCGGCCACCTTCGCAACAAGgtggggggggggttgcggggtt
ggggagggggtgggtggcagctgttgacactaaagctatggctgcccctggtgccaaatgttgaggggaccaaggcaggccgattgctgagtttgag
acagcctggtctacagagagagttttaggactacacagagaaaccctgtctggaaaaacaaacaaacaagcaaacaaagagtgaaataatggtgc
atgcctgtatttccacagtgctagggctgaaatgaaggatctgccttgccagacaagccccgcccctgagccctcccctaaccgcctctggcccctcag
cccctcagccctttaatccctcagctctgggttctttctcaagcactttcttgagtgagaaaaacaaattatatcttcagaatttttgaaaatcaatgagg
aaaaaaataggtaaaatgacatcaactcaactttatttcccaaacaattttgttcccaaaagaccagagaggccaatgaccgaccacctttaacccaa
tgagtttccttcagggaccagagagaagtctctgttgtttgggtaattagataatccttcggctgcctgaaagaactgcgtttctaagagagttcaccaa
attgcagattggcttccatgggcttctccttctctacttggagtcatgacacactgtatttatagacagcttgatcaagtggtactttctcttcgcacacaa
caccagcttgatttactgctaaggaaatagtgcaaaaaaagatgagtaaaagaaaaactatcttcagtcttcgacaaacgattttcgcaataggagat
gggcctattacgattgcagttattacagtcactggcatcacatagcatgtacacacacgcgcgcgcgcgcgcgcacacacacacacacacacacaca
cacacacacacacacacacacaccccttaattgccttccacttaaaacgccagacgccaagtcagagacgaaatctcttcaataagctttttcctccct
ccttacaaattattctggcgccacctagtggccaaggtgcagtttgcagttttacaacgtggcgtccaaacaggcacttccgggacacgaaggtaatcc
ctgcaaggtgtgtatccttttgtcccatagatgtgcagctttcctttacccaacaaagccagtgtaataaagccatttgactccaacaagtgctatcttaat
aagagaattatctttatgctgggagtgatggcacacacctttaatcccaaccctccagaggcagaggcagatggatctctgtgagtttgaggactgcct
ggtctacataatgagttccaggtcaagccagtgcgacatccccacaagcatcccaaatggcctgggtgggagagcatgcaggtcacgtcaccagtgc
tctctgactttctccagTCTGGCTGCCACGTGGAATTGTTGTTCCTACGCTACATCTCAGACTGGGACCTGGACCCGGGC
CGGTGTTACCGCGTCACCTGGTTCACCTCCTGGAGCCCGTGCTATGACTGTGCCCGGCACGTGGCTGAGTTTCTGA
GATGGAACCCTAACCTCAGCCTGAGGATTTTCACCGCGCGCCTCTACTTCTGTGAAGACCGCAAGGCTGAGCCTGA
GGGGCTGCGGAGACTGCACCGCGCTGGGGTCCAGATCGGGATCATGACCTTCAAAGgtgagacttgcacactggagaga
gcggtctgagttgccactcagagtgagtgtcagcggggaaactgggggtggggtgctacttaaagaccttcagttcgtcctggatatcaaaagtattac
tttattttttgaggtaggatctcgctatcccaggctgaccttcaacttgcaattctccgacctctgccttctgagtggcggaattacaagtatacatcaatc
tcagaattatcagaatttgagagatagaagttggcagggctacaggtgcgctcagtggcagaactctggtccagcatgtgcaaagccctgcattccac
ctttagcagtcaaataataaattgaggagggagaggaggaggatagtggtcagagagatggttccgtgggggcccttgcctttgtaccttaagtttaa
cccctaaaacactctgactttctgaccttcacctacacacacacacacacacacacacacacacacacacacacctccttcttatttatctatttattttt
cttttaagACTATTTTTACTGCTGGAATACATTTGTAGAAAATCGTGAAAGAACTTTCAAAGCCTGGGAAGGGCTACA
TGAAAATTCTGTCCGGCTAACCAGACAACTTCGGCGCATCCTTTTGgtaagtctgcctgtctgtctgcctgtctctctgtctgtctctg
tctctctgtctgtctctgtctctctctctctctctcatacacacacacatacatacactcacacacacacacacacacctggagcctcttagttatttgttt
gtattatgcattattttatacaatgattacttcaaggcacttacaacccagttttcttttctgctttacccaggacagagcttccacttagacgcttgcctc
ttgcctcctcttcgctcagtcttcataactctttccttttgctaacctcccctcaggtggggttccttccagggcagaattcgccccttctttttttcctgg
tcctcaagcaatttactttcctctggagccacccacttcgtttagacactttcctttccagagatcaaatttaaagcccttcactccgtttatatcatctct
ctttctccacagCCCTTGTACGAAGTCGATGACTTGCGAGATGCATTTCGTATGTTGGGATTTTGAAAGCAACCTCCTGGAATG
TCACACGTGATGAAATTTCTCTGAAGAGACTGGATAGAAAAACAACCCTTCAACTACATGTTTTTCTTAAGTAC
TCACTTTTATAAGTGTAGGGGGAAATTATATGACTTTTTAAAAAATACTTGAGCTGCACAGGACCGCCAGAGCAAT
GATGTAACTGAGCTTGCTGTGCAACATCGCCATCTACTGGGGAACAGCAGAACTTCCAGACTTTGGGTCGTGAAT
GATGCTCTTTTTTTTCAACAGCATGGAAAAGCATATGGAGACGACCACACAGTTTGTTACACCCACCCTGTGTTCCT
TGATTCATTTGAATTCTCAGGGGTATCAGTGACGGATTCTTCTATTCTTTCCCTCTAAGGCTCACTTTCAGGGGTCCT
TTTCTGACAAGGTCACGGGGCTGTCCTACAGTCTCTGTCTGAGCAATCACAAGCCATTCTCTCAAAAGCATTAATAC
TCAGGCACATGCTGTATGTTTTCACTGTCCGTCGTGTTTTTCACATTTGTATGTGAAAGGGCTTGGGGTGGGATTTG
AAGAATGCACGATCGCCTCTGGGTGATTTCAATAAAGGATCTTAAAATGCAGATGAGGACTACGAAGAAATCACT
CTGAAAATGAGTTCACGCCTCAAGAAGCAAATCCCCTGGAAACACAGACTCTTTTTCATTTTTAATGTCATTAGTTT
ACTCACAGTCTTATCAAGAAGAAGAGTTCAAGGGTTCAACCCAATTTTCAGATCGCGTCCCTTAAACATCAGTAATT
CTGTTAAAGGGATCAAACATCCTTATTTCTTAACTAACTGGTGCCTTGCTGTAGAGAAAGGAGCAAAGCGCCCAGA
TCCAAAGTATATAGTTATCATAGCCAGGAACCGCTACTCGTTTTCCATTACAAATGGCAAATTCTTCCCCGGGCTCT
CCTCATAGTGCCTGAGACGGACCACGGAGGTGATGAACCTCCGGATTCTCTGGCCCAACACGGTGGAAGCTCTGC
AAGGGCGCAGAGACAGAATGCGGCAGAAATTGCCCCCGAGTCCCAACTCTCCTTTCCTTGCGACCTTGGGAACAA
GACTTAAAGGAGCCTGTGACTTAGAAACTTCTAGTAATGGGTACCTGGGAGTCGTTTGAGTATGGGGCAGTGATT
TATTCTCTGTGATGGATGCCAACACGGTTAAACAGAATTTTTAGTITTTATATGTGTGTGATGCTGCTCCCCCAAATT
GTTAACTGTGTAAGAGGGTGGCAAAATAGGGAAAGTGGCATTCACCTATAGTTCCAGCATTCAGGAAGCTGAGGC
AGGAGGATTGTAAATTTGAGGCCAGTCTGAGCTGTAAGGTGAGACCCTATTTCAAACAACACAGCCAGAATTGGG
TTCTGGTAAATCATACTTAACAAGGGAAAAATGCAAGACGCAAGACCGTGGCAAGGAAATGACGCTTTGCCCAAC
GAAATGTAGGAAACCAACATAGACTCCCAGTTTGTCCCTCTTTATGTCTGGTCTCCCTAACAACGATCTTTGCTAAT
GAGAAAAATATTAGAAAAAAATATCCCTGTGCAATTATCACCCAGTCGCCATTATAATGCAATTAAAAGGCCCACA
AGAAATCCTGTATACACGACCGTTATTTATTGTATGTAAGTTGCTGAGGAAGAGGAGAAAAAAATAAAGATCATCC
ATTCCTTCCTGCAtctatccctgttttttatgttgctgcgtggcatctattctgaaatattaaagtgggtgcctgaagtttcataaatttgaaactttag
agattactatatatctgcactcgtcattgtgatcatccaaaatcgtaatgattatggctcggcagctgtgctcttgatttttagcaactcccacccccaccc
ccacccccacccccacccccaacccccaccctgcgtgcagcaagttcatcctggcttattttaaatcaactgaattcgagattaaaatgtgaaagttttg
gagatgaactactgaataaaatgatgtcgggaaaaagcatttatatattaaagtcatacagatcacagggaaggtggcgcatgtatttaacccccag
cattggaaagatggaggcaggaggctctctgtgggtttgaggtcagcctgatctagacagagtgctccgcagatagccacacagagagagcctgcct
tagagaaataaatacctgatgaaatagaattgaattgagagtccagaaattaacccactcagctatgaccaactgatttcagataaaggtccaaggt
gtactgagtcagcaaaccctgctggggcaatctgacatcaggtgcaaagagtgcagtccacatggtgacacctgcctgtcccctactcgggaggctga
gacaagaagatcagtagtagttgcagtaaatctccacccaaatatgccctggcaatgaaaacacaactcaattaatatgaatacatgctgtgcgccta
gattgggcagatctaccgctgcactaccatcttctccatctatgagaccctttagaacttgcggtttctaaggtttgggggtataattagccccagggcta
tccacaacactgtcctaggcgcatttcctaaacacgagcttattcataagcccagccagagggttcacattgcccacaacacaccctcctttcctaccac
ataaccaaagcccaaactctagaactggttctaactgggaattctcatggcatcccatagcatatacccccttctctgcagtgagcaatatgtccagtat
ttcctggaaaccattggtacacaaaactctgagtcaccaacacccgctgctctgtctactgaactggcttccaatgttaactaattcatttgagtgtgtgt
attagtgtgagtgtgtgttagttacttttgctgttgttgtgattaaaacaccatgaccaagggcaacctaaggaagagagcgtgtgtatcttggcttatgc
cactgagaacgaagrcatcactgtggggtggaggcatggcttcaagtgtcaggcatgacgtgaggagcaggaagctgggagatcacatctttaacag
cgagtgccaagcagagagggaaactggaagttaaagcccacaagtgatgtgctccctcagccaggctgcacttcctgaacaccccaaaacagcgcc
acctacctagaaccgactttgaatatctgagcctatggggacatttgtcattctaaccactgttgtgcactgttgttgcacagtgagccatcttgccagct
catattccacaatttgtatttcattttaccaatgctctctctgtagtagtgataatgatgactgttcccttttttggttttgcttcgttttgagatttcagt
atttttctcaagttttattttaagtgatgttaattacagcgtttgaaggggaggagctaattccactcaaaatggaagactctataatgtacccattaaact
gctaaaaaaaaaataataataataatggtaagtctacaagaggagtcagtttagacccctagtgttgtcagagtgtgaccacaatcacctgcccagatca
gagccagagaacccggaagctatttcatactctggtgcaatggggggggggggggggggagaaattttaaaaaaacaaaaaggaggaagaaaaa
cacacacacaacacaaggaagaattaagtcctgattgactgactccatcttgcccaccctctccaccctaaaatggcacaaaagaaaataccacacc
taaagactacttttggtgtaaaacaggtaactgatgggctaggatgggaacagggtatgatgatctgtctaaaaaaatgttcctttcacgaaggtgtgt
acgtacttctgagcagataggatcgggacaccagggttcaatgcttgggaagtcacaatttcatctggggactggatacagatttacaaagggtccac
acattcccagcttccatttgcagcctggcatctctagaggctcctccccaagccccaacccacacctacagctagaaaggaccctttctggaatggggt
ttctgctgtacctctgaaatggtaaacaccttaaagctgagtcatccttagcctggagaggcattcatcaactctcgcatccccaacatacaatattaaa
agtccactaaattggtagctatgttgcaaaatagttcaaaattaacgattttacaatattcatttatgcttgaaattctagtcctaagccaagcttgtgtct
gccagcattgatgttcttgcgtccagtagggctgacaatgtcagtttgatacctggttttaggatctgagtgtaccctaagccaatcaggctggagttgtt
cactttgccagaaaagcaggcatcagggtggaactgaaatttggctgctattccaaagcgagtgttactgttttctgcagtccaggcgagattgacagc
agtctccaacttcttgttcgccttctggtaaatggaaccaccaaactctgtcccgtcgtatgaagctgtcttgctctgggtcactcaggacttcgaggtctc
aaaattcatctggtagccagctagccaaccctcatagccaagcaccggattgagggcccagcgatgtcaaagtccacgccacagcccaagttgatgt
gctccctcttgtaccctgtcatgatttttagcattttcccccaagttttgggtgaaaaagatgaatcgaaggtcagcttcagtccacaagcaagctggtct
cccacggtgatctcagtgcccagggtgctgtctgtgttccacttctccgtaaatgtttgcccatactcagtccatctgtccttggtttccagactgccgttc
actctggtggtctccgtgttggcagagcctgagctggtaaattccaatctattcttggacttcgttttcaaatcaagttttattaagccaaagcggtagcc
cttggtgaagacatccctgatggatagaggcagctgcgatggggggttgcagcgaggatgctgggagcgcagcgaataggcagagggcggggcag
ctctcacgattgtttcttaagaagacttcctttaaaattaatactaatccactaactactcactcattcttccaggattttactgatcaattgctgtatacg
catagcgccgcggtcatcgttacacagacgtgttaagcacacaaagactgctttgaagaaggctgaaagatctcggggctggagagagaactctgcag
tttacagagcttcttggtcctccagagaacccaatttcagttcccagcatccacatcacacagctcacaaccgccggaaactccagctccagagggtcc
aacacccctgttctggcctccaggagcacctacatacatgtgtcatgcaaacacacacacaaacacacaacacacacatacatacataaattaaaaa
tatatataaataaatcaatcctttttttttaaagcagtcttaaaatctgtggacctagagaagtattatctgaaattttgaaatgggacccaaagaacgt
cttctcacaggaactaatacttacagtcttttgaagcataggtaaatgttcaatcggtgatgataaacctagagactgagactgcagccaggctggga
gaggacttgtccagcatgcgctaagtccagtgctcagcccac-3′
SEQ ID NO: 24
PRIMER 1:
5′ACAATAATAATCAGAGCTGAAGGAAGACTATGGTGACAGAGAAGCCTTGCCCTGACTTTCTTCTCCAACTCACAG
CTGTGACGGAAGATCACTTCG3′
SEQ ID NO: 25
PRIMER 2:
5′CACCAGGGGCAGCCATAGCTTTAGTGTCAACAGCTGCCACCCACCCCCTCCCCAACCCCGCAACCCCCCCCCCACC
TGAGGTTCTTATGGCTCTTG3′
SEQ ID NO: 26
PRIMER 3:
:5′CCCACAAGCATCCCAAATGGCCTGGGTGGGAGAGCATGCAGGTCACGTCACCAGTGCTCTCTGCTCTTTCTCCA
GCTGTGACGGAAGATCACTTCG3′
SEQ ID NO: 27
PRIMER 4:
5′CCCACCCCCAGTTTCCCCGCTGACACTCACTCTGAGTGGCAACTCAGACCGCTCTCTCCAGTGTGCAAGTCTCACC
TGAGGTTCTTATGGCTCTTG3′
SEQ ID NO: 28
PRIMER 5:
5′ACACACACACACACACACACACACACACACACACACACACCTCCTTCTTATTTATCTATTTATTTTTCTTTTAA [symbol] TG
TGACGGAAGATCACTTCG3′
SEQ ID NO: 29
PRIMER 6:
5′GAGAGAGAGAGAGACAGAGACAGACAGAGAGACAGAGACAGACAGAGAGACAGGCAGACAGACAGGCAGAC
TTACCTGAGGTTCTTATGGCTCTTG3′
SEQ ID NO: 30
PRIMER 7:
5′GTTTAGACACTTTCCTTTCCAGAGATCAAATTTAAAGCCCTTCACTCCGTTTATATCATCTCTCTTTCTCCACA [symbol] TG
TGACGGAAGATCACTTCG3′
SEQ ID NO: 31
PRIMER 8:
5′CCAGTAGATGGCGATGTTGCACAGCAAGCTCAGTTACATCATTGCTCTGGCGGTCCTGTGCAGCTCAAGTATTTT
CTGAGGTTCTTATGGCTCTTG3′
SEQ ID NO: 32
PRIMER 9:
5′CCCACAAGCATCCCAAATGGCCTGGGTGGGAGAGCATGCAGGTCACGTCACCAGTGCTCTCTGCTCTTTCTCCAG
AACGGCTGCCACGCTGAGATGCTCTTCCTGCG3′
SEQ ID NO: 33
PRIMER 10:
5′CCCACCCCCAGTTTCCCCGCTGACACTCACTCTGAGTGGCAACTCAGACCGCTCTCTCCAGTGTGCAAGTCTCACC
TTTGTAGCTCATGACAGACAGTC3′
SEQ ID NO: 34
PRIMER 11:
5′AGGCAAAGCCTCCATCCAGACAGGCAGCCAGCACTACTGGAGCACATGCACAAGCAGATGAGACTGTCTTGTTA
C3′
SEQ ID NO: 35
PRIMER 12:
5′GTTTAGACACTTTCCTTTCCAGAGATCAAATTTAAAGCCCTTCACTCCGTTTATATCATCTCTCTTTCTCCACA [symbol] CG
CCGTACGACATGGAGG3′
SEQ ID NO: 36
PRIMER 13:
5′AGGCAAAGCCTCCATCCAGACAGGCAGCCAGCACTACTGGAGCACATGCACAAGCAGATGAGACTGTCTTGTTA
CTTAAAGCCCAAGTAGAACAAACACTTC3′
SEQ ID NO: 37
PRIMER 14:
5′TATGACTGTGCCCGGCACGTGGCTGAGTTTCTGAGATGGAACCCTAACCTCAGCCTGAGGATTTTCACCGCGCGC
CTGTGACGGAAGATCACTTCG3′
SEQ ID NO: 38
PRIMER 15:
5′CAGTGTGCAAGTCTCACCTTTGAAGGTCATGATCCCGATCTGGACCCCAGCGCGGTGCAGTCTCCGCAGCCCCTC
CTGAGGTTCTTATGGCTCTTG3′
SEQ ID NO: 39
PRIMER 16:
5′TATGACTGTGCCCGGCACGTGGCTGAGTTTCTGAGATGGAACCCTAACCTCAGCCTGAGGATTTTCACCGCGCGC
CTCTATTTCTGCGAGGAGCG3′
SEQ ID NO: 40
PRIME R17:
5′CTTTGAAGGTCATGATCCCGATCTGGACCCCAGCGCGGTGCAGTCTCCGCAGCCCCTCCGGCTCCGCGTTGCGCT
CCT3′
SEQ ID NO: 41
CTCTACTTCTGTGAGGACCGCAAGGCTGAGCCC
SEQ ID NO: 42
CTCTACTTCTGTGAAGACCGCAAGGCTGAGCCT
SEQ ID NO: 43
CTCTACTTCTGTGAAGATCGCAAGGCTGAGCCT
SEQ ID NO: 44
CTTATTTCTGCGAGGAGCGCAACGCGGAGCCG
SEQ ID NO: 45
CTCTACTTCTGTGACGAGGAGGACAGTCAAGAGAGA
SEQ ID NO: 46
CTGTACTTCTGTGATGAAGAGGACAGCGTGGAGAGA
SEQ ID NO: 47
LYFCEDRKAEP
SEQ ID NO: 48
LYFCEDRKAEP
SEQ ID NO: 49
LYFCEDRKAEP
SEQ ID NO: 50
LYFCEERNAEP
SEQ ID NO: 51
LYFCDEEDSQER
SEQ ID NO: 52
LYFCDEEDSVER
SEQ ID NO: 53
Nucleotide sequence encoding the mouse AID mutant (Xenopus exon 3)
Underlined nucleotides indicate exon 3 sequence from Xenopus; other nucleotides are mouse.
ATGGACAGCCTTCTGATGAAGCAAAAGAAGTTTCTTTACCATTTCAAAAATGTCCGCTGGGCCAAGGGACGGCAT
GAGACCTACCTCTGCTACGTGGTGAAGAGGAGAGATAGTGCCACCTCCTGCTCACTGGACTTCGGCCACCTTCGCA
ACAAGAACGGCTGCCACGCTGAGATGCTCTTCCTGCGCTACCTGTCTATATGGGTGGGTCACGACCCCCATAGGAA
CTACCGGGTCACGTGGTTCAGCTCCTGGAGCCCCTGCTATGACTGTGCCAAGCGCACCCTCGAGTTCTTAAAGGGG
CACCCCAACTTCAGTCTGCGCATCTTCAGCGCCAGGCTCTATTTCTGCGAGGAGCGCAACGCGGAGCCGGAGGGG
CTGCGGAAACTGCAGAAAGCGGGGGTGCGACTGTCTGTCATGAGCTACAACTATTTTTACTGCTGGAATACATTTG
TAGAAAATCGTGAAAGAACTTTCAAAGCCTGGGAAGGGCTACATGAAAATTCTGTCCGGCTAACCAGACAACTTC
GGCGCATCCTTTTGCCCTTGTACGAAGTCGATGACTTGCGAGATGCATTTCGTATGTTGGGATTTTGA
SEQ ID NO: 54
Amino acid sequence for the mouse AID mutant (Xenopus exon 3)
Underlined amino acids indicate exon 3 sequence from Xenopus; other amino acids are mouse.
M D S L L M K Q K K F L Y H F K N V R W A K G R H E T Y L C Y V V K R R D S A T S C S L D F G H L R N
K N G C H A E M L F L R Y L S I W V G H D P H R N Y R V T W F S S W S P C Y D C A K R T L E F L K G H
P N F S L R I F S A R L Y F C E E R N A E P E G L R K L Q K A G V R L S V M S Y N Y F Y C W N T F V E
N R E R T F K A W E G L H E N S V R L T R Q L R R I L L P L Y E V D D L R D A F R M L G F
SEQ ID NO: 55
Nucleotide sequence encoding the mouse AID mutant (Xenopus active-site loop)
Underlined nucleotides indicate active-site loop-encoding sequence from Xenopus; other nucleotides
are mouse.
ATGGACAGCCTTCTGATGAAGCAAAAGAAGTTTCTTTACCATTTCAAAAATGTCCGCTGGGCCAAGGGACGGCAT
GAGACCTACCTCTGCTACGTGGTGAAGAGGAGAGATAGTGCCACCTCCTGCTCACTGGACTTCGGCCACCTTCGCA
ACAAGTCTGGCTGCCACGTGGAATTGTTGTTCCTACGCTACATCTCAGACTGGGACCTGGACCCGGGCCGGTGTTA
CCGCGTCACCTGGTTCACCTCCTGGAGCCCGTGCTATGACTGTGCCCGGCACGTGGCTGAGTTTCTGAGATGGAAC
CCTAACCTCAGCCTGAGGATTTTCACCGCGCGCCTCTATTTCTGCGAGGAGCGCAACGCGGAGCCGGAGGGGCTG
CGGAGACTGCACCGCGCTGGGGTCCAGATCGGGATCATGACCTTCAAAGACTATTTTTACTGCTGGAATACATTTG
TAGAAAATCGTGAAAGAACTTTCAAAGCCTGGGAAGGGCTACATGAAAATTCTGTCCGGCTAACCAGACAACTTC
GGCGCATCCTTTTGCCCTTGTACGAAGTCGATGACTTGCGAGATGCATTTCGTATGTTGGGATTTTGA
SEQ ID NO: 56
Amino add sequence for the mouse AID mutant (Xenopus active-site loop)
Underlined amino adds indicate active-site loop-encoding sequence from Yenopus; other amino adds
are mouse.
M D S L L M K Q K K F L Y H F K N V R W A K G R H E T Y L C Y V V K R R D S A T S C S L D F G H L R N
K S G C H V E L L F L R Y I S D W D L D P G R C Y R V T W F T S W S P C Y D C A R H V A E F L R W N P
R N L S L R I F T A L Y F C E E R N A E P E G L R R L H R A G V Q I G I M T F K D Y F Y C W N T F V E
N R E R T F K A W E G L H E N S V R L T R Q L R R I L L P L Y E V D D L R D A F R M L G F
SEQ ID NO: 57
Nucleotide sequence encoding the mouse AID mutant (Catfish active-site loop)
Underlined nucleotides indicate active-site loop-encoding sequence from Catfish; other nucleotides are
mouse.
ATGGACAGCCTTCTGATGAAGCAAAAGAAGTTTCTTTACCATTTCAAAAATGTCCGCTGGGCCAAGGGACGGCAT
GAGACCTACCTCTGCTACGTGGTGAAGAGGAGAGATAGTGCCACCTCCTGCTCACTGGACTTCGGCCACCTTCGCA
ACAAGTCTGGCTGCCACGTGGAATTGTTGTTCCTACGCTACATCTCAGACTGGGACCTGGACCCGGGCCGGTGTTA
CCGCGTCACCTGGTTCACCTCCTGGAGCCCGTGCTATGACTGTGCCCGGCACGTGGCTGAGTTTCTGAGATGGAAC
CCTAACCTCAGCCTGAGGATTTTCACCGCGCGCCTCTACTTCTGTGACGAGGAGGACAGTCAAGAGAGAGAGGGG
CTGCGGAGACTGCACCGCGCTGGGGTCCAGATCGGGATCATGACCTTCAAAGACTATTTTTACTGCTGGAATACAT
TTGTAGAAAATCGTGAAAGAACTTTCAAAGCCTGGGAAGGGCTACATGAAAATTCTGTCCGGCTAACCAGACAAC
TTCGGCGCATCCTTTTGCCCTTGTACGAAGTCGATGACTTGCGAGATGCATTTCGTATGTTGGGATTTTGA
SEQ ID NO: 58
Amino acid sequence for the mouse MD mutant (Catfish active-site loop)
Underlined amino acids indicate active-site loop-encoding sequence from Catfish other amino acids are
mouse.
M D S L L M K Q K K F L Y H F K N V R W A K G R H E T Y L C Y V V K R R D S A T S C S L D F G H L R N
K S G C H V E L L F L R Y I S D W D L D P G R C Y R V T W F T S W S P C Y D C A R H V A E F L R W N P
R N L S L R I F T A L Y F C D E E D S Q E R E G L R R L H R A G V Q I G I M T F K D Y F Y C W N T F V
E N R E R T F K A W E G L H E N S V R L T R Q L R R I L L P L Y E V D D L R D A F R M L G F
SEQ ID NO: 59
PRIMER 18:
5′AGGCGAATTCTCCATGAAAGTCAGGCTGGC3′
SEQ ID NO: 60
PRIMER 19:
5′GTTAGAATGACGATATCGGATCCATGCTAGTCTGGAAATCTC3′
SEQ ID NO: 61
PRIMER 20:
5′TGGATCCGATATCGTCATTCTAACCACTGTTGTGCAC3′
SEQ ID NO: 62
PRIMER 21:
5′AGGCACGCGTCTAAACTGACTCCTCTTGTAGAC3′
SEQ ID NO: 63
PRIME R22:
5′AGGCGAATTCTCCATGAAAGTCAGGCTGGC3′
SEQ ID NO: 64
PRIMER 23:
5′AGGCACGCGTCTAAACTGACTCCTCTTGTAGAC3′
SEQ ID NO: 65
PRIMER 24:
5′AGGCGAATTCTTTCTTAGACGTCAGGTGGCAC3′
SEQ ID NO: 66
PRIMER 25:
5′AGGCACGCGTCGATACGCGAGCGAACGTGA3′
SEQ ID NO: 67
5′homology arm
TATGACTGTGCCCGGCACGTGGCTGAGTTTCTGAGATGGAACCCTAACCTCAGCCTGAGGATTTTCACCGCGCGC
SEQ ID NO: 68
3′homology arm
GAGGGGCTGCGGAGACTGCACCGCGCTGGGGTCCAGATCGGGATCATGACCTTCAAAG [symbol]
indicates data missing or illegible when filed

1. A transgenic vertebrate of a non-human species or vertebrate cell of a non-human species whose genome comprises
(a) an immunoglobulin (Ig) locus comprising unrearranged or rearranged Ig gene segments positioned upstream of a constant region, wherein said unrearranged Ig gene segments comprise (ia) at least one V segment, at least one D segment, and at least one J segment, or (ib) at least one V segment and at least one J segment; wherein said rearranged Ig gene segments comprise (iia) a joined VDJ segments or (iib) joined VJ segments;
(b) a first expressible gene encoding a first activation-induced deaminase (AID); and
(c) a second expressible gene encoding a second AID, wherein the first and second AIDs are not identical.

2. The transgenic vertebrate or cell of claim 1, said genome comprising a transgene comprising unrearranged V, D, and J segments comprising at least one human V segment, at least one human D segment, and at least one human J segment, or a transgene comprising unrearranged V and J segments comprising at least one human V segment and at least one human J segment; or said transgene comprising a rearranged human VDJ or a rearranged human VJ.

3. The vertebrate or cell of claim 2, wherein (i) said vertebrate is a mouse and said constant region comprises a mouse Sμ switch or a mouse Sμ switch and a mouse Cμ segment; or (ii) said vertebrate is a rat and said constant region comprises a rat Sμ switch or a rat Sμ switch and a rat Cμ segment.

4. The transgenic vertebrate or cell according to claim 2, wherein said vertebrate or cell comprises a mouse vertebrate or mouse cell and said transgene comprises a repertoire comprising functional human IgH V, D and J segments positioned upstream of a mouse constant region, or said vertebrate or cell comprises a rat vertebrate or rat cell and said transgene comprises a repertoire comprising functional human IgH V, D and J segments positioned upstream of a rat constant region.

5. The vertebrate or vertebrate cell of claim 4, wherein said mouse constant region comprises a mouse Sμ switch or comprises a mouse Sμ switch and a mouse Cμ segment, or wherein said constant region comprises a rat Sμ switch or comprises a rat Sμ switch and a rat Cμ segment.

6. The vertebrate or cell of claim 1, wherein either (i) the vertebrate is a mouse, the constant region is a mouse constant region, and one of said expressible AID is a mouse AID; or (ii) the vertebrate is a rat, the constant region is a rat constant region, and one of said expressible AID is a rat AID.

7. The vertebrate or cell of claim 6, wherein (i) said mouse AID and said mouse constant region are derived from the same mouse strain, or wherein (ii) wherein said rat AID and said rat constant region are derived from the same rat strain.

8. The vertebrate or cell of claim 1, wherein said first AID gene comprises a wild-type AID gene.

9. The vertebrate or cell of claim 1, wherein
(i) the vertebrate is a mouse or a rat, or the vertebrate cell is a mouse cell or a rat cell and said first expressible gene encodes a human AID and said second expressible gene encodes a chicken AID; or
(ii) the vertebrate is a mouse or a rat, or the vertebrate cell is a mouse cell or a rat cell and said first expressible gene encodes a human AID and said second expressible gene encodes an African clawed frog AID; or
(iii) the vertebrate is a mouse or a rat, or the vertebrate cell is a mouse cell or a rat cell and said first expressible gene encodes a human AID and said second expressible gene encodes mouse AID; or
(iv) the vertebrate is a mouse or a rat, or the vertebrate cell is a mouse cell or a rat cell and said first expressible gene encodes a human AID and said second expressible gene encodes rat AID; or
(v) said second expressible gene encodes a chimaeric AID; or
(vi) the vertebrate is a mouse, or the vertebrate cell is a mouse cell and said first expressible gene encodes a mouse AID and said second expressible gene encodes a chimaeric AID; or
(vii) the vertebrate is a rat, or the vertebrate cell is a rat cell and said first expressible gene encodes a rat AID and said second expressible gene encodes a chimaeric AID.

10. The vertebrate or cell of claim 9, wherein in any one of (iii), (iv), (vi) or (vii), said gene encoding said mouse or rat AID comprises an endogenous gene.

11. The transgenic vertebrate or vertebrate cell of claim 1, wherein each said first and said second gene encodes a human AID.

12. The vertebrate or cell of claim 11, wherein said first AID gene comprises the nucleotide sequence of a human AID, human APOBEC1, human APOBEC3C, human APOBEC3F, human APOBEC3G, or a nucleotide sequence that is at least 95% identical thereto.

13. The vertebrate or cell of claim 11, wherein said constant region comprises a human constant region.

14. The vertebrate or cell of claim 2, wherein said transgene comprises at least one human IgH V segment, at least one human D segment and at least one human J segment.

15. The vertebrate or cell of claim 18, wherein said transgene comprises a plurality human IgH V segments, a plurality of human D segments and a plurality of human J segments, or said transgene comprises substantially the full human repertoire of functional IgH V, D and J segments.

16. A transgenic vertebrate or cell according to claim 2, comprising
said transgene comprising a repertoire comprising functional human IgH V, D and J segments positioned upstream of a constant region, wherein the constant region is a mouse or rat constant region, wherein each said expressible AID gene comprises a human AID gene.

17. The transgenic vertebrate cell of claim 16, wherein when said constant region is a mouse constant region, it comprises a mouse Sμ switch or a mouse Sμ switch and a mouse Cμ segment, and wherein when said constant region is a rat constant region, it comprises a rat Sμ switch or a rat Sμ switch and a rat Cμ segment.

18. The vertebrate or cell according to claim 2, wherein
said human segment comprises a human heavy (IgH) chain locus V segment, and said vertebrate or cell further comprises a transgene comprising (a) at least one human Igκ V segment or (b) at least one human Igλ V segment and also comprises at least one human J segment.

19. The vertebrate or cell according to claim 2, wherein
(i) said transgene comprising a plurality of human IgH V, D and J segments constituting a repertoire comprising functional human IgH V, D and J segments; and
(ii) said vertebrate or cell further comprises human immunoglobulin light (IgL) chain segments, said human IgL segments comprising one or both of a repertoire comprising functional human Igκ V and J segments and a repertoire comprising functional human Igλ V and J segments.

20. The vertebrate or cell of claim 2, wherein said first AID is human AID and said second AID comprises an amino acid sequence that is at least 95% identical to SEQ ID NO: 12; or wherein said first AID is selected from the group consisting of human APOBEC1, human APOBEC3C, human APOBEC3F and human APOBEC3G, and said second AID comprises an amino acid sequence that is at least 95% identical to an amino acid sequence selected from the group consisting of human APOBEC1, human APOBEC3C, human APOBEC3F and human APOBEC3G.

21. The vertebrate or cell of claim 2, wherein each said AID comprises an amino acid sequence that is at least 95% identical to SEQ ID NO: 12; or each said AID comprises an amino acid sequence that is at least 95% identical to an amino acid sequence selected from the group consisting of human APOBEC1, human APOBEC3C, human APOBEC3F or human APOBEC3G.

22. The vertebrate or cell according to claim 2, wherein said transgene comprises at least one human λ segment and at least one human Cλ segment.

23. The vertebrate or cell of claim 22, wherein said at least one human Cλ segment comprises one or both of C_{λ}6 and C_{λ}7.

24. The vertebrate or cell according to claim 22, wherein the transgene comprises a plurality of human Jλ segments.

25. The vertebrate or cell of claim 24, wherein said plurality of Jλ segments comprises at least two of J_{λ}1, J_{λ}2, J_{λ}6 and J_{λ}7.

26. The vertebrate or cell according to claim 22, wherein the transgene comprises at least one human J_{λ}-C_{λ} cluster.

27. The vertebrate or cell of claim 26, said cluster comprising at least J_{λ}7-C_{λ}7.

28. The vertebrate or cell according to claim 22, wherein the transgene comprises a human Eλ enhancer.

29. The vertebrate or cell according to claim 22, wherein the vertebrate or cell comprises a further transgene, the further transgene comprising at least one human IgH V segment, at least one human D segment and at least one human J segment, or said further transgene comprises a human repertoire comprising functional IgH V, D and J segments.

30. The vertebrate or cell of claim 1, wherein the expression of at least one of the AIDs is inducible.

31. The vertebrate or cell of claim 1, wherein said first and second AID genes are present in the genome under operable control of wild-type AID gene control elements.

32. The vertebrate or cell of claim 1, wherein at least one V, D and/or J segment sequence in the transgene has been codon-optimised for AID.

33. The vertebrate or cell of claim 32, wherein said V, D and/or J sequence comprises a sequence motif selected from the group consisting of DGYW, WRC, WRCY, WRCH, RGYW, AGY,TAC, WGCW, wherein W=A or T, Y=C or T, D=A, G or T, H=A or C or T, and R=A or G.

34. A cell according to claim 1, said cell comprising a B-cell, a hybridoma cell, a stem cell, an embryonic stem cell or a haematopoietic stem cell.

35. A method of isolating an antibody or nucleotide sequence encoding said antibody, the method comprising
(a) immunising a vertebrate according to claim 1 with an antigen such that the vertebrate produces antibodies; and
(b) isolating from the vertebrate an antibody that specifically binds to said antigen and/or a nucleic acid comprising a sequence encoding at least the heavy and/or the light chain variable regions of said antibody.

36. The method of claim 35, further comprising the step of joining said isolated nucleic acid comprising said sequence encoding said variable region of said antibody to a nucleic acid comprising a sequence encoding a human constant region.

37. An antibody produced by the method of claim 35.

38. A chimaeric AID protein comprising a mouse or rat AID and comprising a heterologous active-site loop from one of a human, chicken, bird, fish, reptile, Xenopus, catfish or zebrafish AID active-site loop.

39. A chimaeric AID comprising an amino acid sequence selected from the group consisting of SEQ ID NO: 54, 56 and 58.


SEQUENCE LISTING (68 sequences)

SEQ ID NO: 1 - DNA, 597 bp, Homo sapiens
atggacagcctcttgatgaaccggaggaagtttctttaccaattcaaaaatgtccgctgggctaagggtcggcgtgagacctacctgtgctacgtagtgaagaggcgtgacagtgctacatccttttcactggactttggttatcttcgcaataagaacggctgccacgtggaattgctcttcctccgctacatctcggactgggacctagaccctggccgctgctaccgcgtcacctggttcacctcctggagcccctgctacgactgtgcccgacatgtggccgactttctgcgagggaaccccaacctcagtctgaggatcttcaccgcgcgcctctacttctgtgaggaccgcaaggctgagcccgaggggctgcggcggctgcaccgcgccggggtgcaaatagccatcatgaccttcaaagattatttttactgctggaatacttttgtagaaaaccacgaaagaactttcaaagcctgggaagggctgcatgaaaattcagttcgtctctccagacagcttcggcgcatccttttgcccctgtatgaggttgatgacttacgagacgcatttcgtactttgggactttga

SEQ ID NO: 2 - DNA, 597 bp, Pan troglodytes
atggacagcctcttgatgaaccggaagaagtttctttaccaattcaaaaatgtccgctgggctaagggtcggcgtgagacctacctgtgctacgtagtgaagaggcgggacagtgctacatccttttcactggactttggttatcttcgcaataagaacggctgccacgtggaattgctcttcctccgctacatctcggactgggacctagaccctggccgctgctaccgcgtcacctggttcacctcctggagcccctgctacgactgtgcccgacatgtggccgactttctgcgagggaaccccaacctcagtctgaggatcttcaccgcgcgcctctacttctgtgaggaccgcaaggctgagcccgaggggctgcggcggctgcaccgcgccggggtgcaaatagccatcatgaccttcaaagattatttttactgctggaatacttttgtagaaaaccatgaaaggactttcaaagcctgggaagggctgcatgaaaattcagttcgtctctccagacagcttcggcgcatccttttgcccctgtatgaggttgatgacttacgagacgcatttcgtactttgggactttga

SEQ ID NO: 3 - DNA, 600 bp, Bos Taurus
atggacagcctcttgaagaagcagagacagtttctttaccagttcaaaaacgtgcgctgggctaagggccgccatgagacctacttgtgctacgtggtgaagcggcgggacagtcccacctccttctcactggacttcgggcaccttcgaaacaaggccggatgccacgtggagttgctcttccttcgctacatctctgactgggatctggaccctgggcggtgctaccgcgtcacctggttcacgtcttggagcccctgctacgactgtgcgcggcacgtggccgacttcctgcgggggtaccccaacctgagcctgcggatcttcacggcgcgcctctacttctgcgacaaggagcgcaaggccgagccagaggggctgcggcggctgcaccgcgctggagtccagatcgccatcatgacgttcaaagattatttttattgctggaatacttttgtggaaaatcatgaaagaactttcaaagcctgggagggactgcatgaaaattcggttcgtctgtctagacagcttcgacgcatccttttgccactctacgaggttgatgacttgcgggatgcatttcgtactttgggactttga

SEQ ID NO: 4 - DNA, 597 bp, Canis lupus
atggacagcctcctgatgaagcagaggaagtttctttaccatttcaagaatgtccgctgggcgaagggtcgccatgagacttacttgtgctacgtggtgaagcggcgggatagtgccacctccttttctctggactttggtcaccttcgaaacaagtcgggctgccacgtggagctgctcttcctccgctacatctccgactgggacctggaccccggccggtgctaccgcgtcacctggttcacgtcctggagcccctgctacgactgcgcgcggcacgtggcggacttcctgcgcgggtaccccaacctcagcctcaggatcttcgccgcgcgcctctacttctgcgaggaccgcaaggcggagcccgaggggctgcggcggctgcaccgggcgggcgtccagatcgccatcatgaccttcaaggattatttttattgctggaatacttttgtggaaaatcgtgaaaaaactttcaaagcctgggaggggttgcacgaaaattccgttcgactatccagacagcttcgacgcattcttttgcccctgtatgaggttgatgacttacgagatgcatttcgtactttgggactttga

SEQ ID NO: 5 - DNA, 621 bp, Oryctolagus cuniculus
atgccgcagacccgctcctcgccgctggtcctccttttgatgaagcagaagaagtttctttatcacttcaagaatgtccgctgggctaagggccggcacgagacctacctgtgctacgtggtcaagcggcgggacagtgccacctccttctcactggacttcggctacctgcgcaacacgaacggctgccacgtggaattgctcttcctccgctacatctccgactgggacctggaccccggccgctgctaccgcgtcacctggttcacctcctggagcccttgctacgactgtgcccggcacgtggctgacttcctgagaggcaaccccaacctcactctgaggatcttcaccgcgcgcctctacttctgcgaggaccgcaaggccgagcccgagggactgcggcggctgcaccaagcgggcgtccagctcggcatcatgaccttcaaagattatttttactgctggaatactttcgtggagaaccgtgagagaacgttcaaggcctgggaaggcctgcatgaaaattctgtccgcctgtccagacagctccggcgcatccttctgcccctttatgaggtcgatgacctacgagatgcgtttcgtactttgggactttga

SEQ ID NO: 6 - DNA, 597 bp, Rattus norvegicus
atggacagcctcttgatgaagcaaaagaagtttctttaccacttcaaaaatgtccgctgggctaagggtcggcacgagacctacctgtgctatgtggtgaagaggagagatagtgccacctccttctcactggactttggccaccttcgcaacaagtcgggctgccacgtggaattgttgttcctacgctacatctcggactgggacctggaccccggccggtgttaccgtgtcacctggttcacttcctggagcccctgctacgactgtgcgcggcacgtggctgagtttctgagatggaaccctaacctcagcctgaggattttcaccgcgcgcctctacttctgcgaagaccgcaaggctgagcctgaggggctgcggaggctgcaccgcgccggagtccagatcgggatcatgaccttcaaagactatttttactgctggaatacatttgtagaaaatcatgaaagaactttcaaagcctgggaagggctgcatgaaaactccgtcaggctaaccagacagcttcggcgcatccttttgcccttgtatgaagtcgatgacttgagagatgcgtttcgtattttgggactttga

SEQ ID NO: 7 - DNA, 597 bp, Mus musculus
atggacagccttctgatgaagcaaaagaagtttctttaccatttcaaaaatgtccgctgggccaagggacggcatgagacctacctctgctacgtggtgaagaggagagatagtgccacctcctgctcactggacttcggccaccttcgcaacaagtctggctgccacgtggaattgttgttcctacgctacatctcagactgggacctggacccgggccggtgttaccgcgtcacctggttcacctcctggagcccgtgctatgactgtgcccggcacgtggctgagtttctgagatggaaccctaacctcagcctgaggattttcaccgcgcgcctctacttctgtgaagaccgcaaggctgagcctgaggggctgcggagactgcaccgcgctggggtccagatcgggatcatgaccttcaaagactatttttactgctggaatacatttgtagaaaatcgtgaaagaactttcaaagcctgggaagggctacatgaaaattctgtccggctaaccagacaacttcggcgcatccttttgcccttgtacgaagtcgatgacttgcgagatgcatttcgtatgttgggattttga

SEQ ID NO: 8 - DNA, 597 bp, Gallus gallus
atggacagcctcttgatgaagaggaagctcttcctctacaatttcaagaacctgcgctgggccaaaggccgtcgtgaaacctacctctgttatgttgtgaagcgccgtgacagtgctacatcatgctccctggactttggatacctgcgtaacaagatgggttgccatgtggaggttctcttcctacgctacatctcagcttgggacctggacccaggccgctgctaccgcatcacatggttcacctcctggagcccctgttatgactgtgcccgacatgtggctgacttccttcgtgcctacccaaacttgaccctccgcattttcactgcccgcctctacttctgtgaagatcgcaaggctgagcctgaggggctgagacgcctgcaccgggctggggcccaaatcgccatcatgactttcaaagatttcttctactgctggaacacgtttgtggagaacagggaaaagacattcaaagcctgggaagggctgcatgaaaactctgtccatctgtccaggaaactccgacggatccttctgccactgtatgaagtagatgatttacgagatgcctttaaaactctgggactttga

SEQ ID NO: 9 - DNA, 606 bp, Xenopus laevis
atgacgatggacagcatgttgttgaagcgcaacaagttcatctatcactacaagaacctgcgctgggcccggggtcggcacgagacctacctgtgctacatagtcaagcggagatacagctcagtgtcctgcgcgttggacttcgggtacctgcggaaccgcaacggctgccacgctgagatgctcttcctgcgctacctgtctatatgggtgggtcacgacccccataggaactaccgggtcacgtggttcagctcctggagcccctgctatgactgtgccaagcgcaccctcgagttcttaaaggggcaccccaacttcagtctgcgcatcttcagcgccaggctctatttctgcgaggagcgcaacgcggagccggaggggctgcggaaactgcagaaagcgggggtgcgactgtctgtcatgagctacaaagattatttctactgctggaacacctttgtggagacccgggagagcggctttgaagcctgggatggattacacgagaactcggtcagactggcccggaagctgcggcgcatcttgcagccgccgtacgacatggaggatctgagagaagtgtttgttctacttgggctttaa

SEQ ID NO: 10 - DNA, 630 bp, Ictalurus punctatus
atgagcaagctggacagtgtgctgctgactcagaggaagtttatttaccactataagaatgtgcgctgggctcgtgggaggaacgagacctacctctgttttgtggtcaagaaacgcaacagtcccgactcgctctccttcgacttcggacacctgcgcaatcgttctggctgccatgtggagcttctcttcctgagctatcttggggtactgtgcccaggtttcttgggttccggtgtggatggtgtcagggtggcttatgccatcacctggttctgttcctggtcaccctgttcaaactgtgcccatcgcctttctcgcttcatgtctcagatgcccaacctgcggctgcgcatcttcgtctcgcgcctctacttctgtgacgaggaggacagtcaagagagagagggactccgttgcttgcagagggcaggtgtgcaagtgacagtcatgacctataaagattttttctactgttggcaaacctttgtggctcaaaatcagaaggctttcaaggcttgggacgaccttcaccagaactctatccgactgtctcggaaactacagcgaatcctgcagcctagtgagtctgaagacctgagggatggcttcgctctgctgggcctttaa

SEQ ID NO: 11 - DNA, 633 bp, Danio rerio
atgatctgcaagctggacagtgtgctcatgacccagaagaaattcatcttccactataagaatgtgcgctgggctcgagggagacacgaaacctacctttgttttgtagtaaagcgacgcatcggccctgattccctctcttttgactttggacacctgcgcaatcgctccggatgccatgtagagcttctctttctgcgtcacttgggtgcgttgtgtccgggcctgagcgcttccagtgtggacggtgcaagattgtgttactcagtgacctggttctgctcctggtctccctgctctaaatgcgctcaacagctcgcccacttcctgtcacagacgcccaatctgaggctgaggatctttgtgtcacgcctgtacttctgtgatgaagaggacagcgtggagagagaaggtctgcgacacctgaagagggcaggagttcagatctcggtcatgacttataaagactttttctactgctggcaaacgtttgttgcaaggagggagcggagttttaaagcctgggatggacttcatgaaaactctgtccggcttgttcgaaaactcaatcggattctgcagccttgcgaaactgaggatctgagggatgtttttgctcttcttgggttatga

SEQ ID NO: 12 - protein, 198 aa, Homo sapiens [sequence omitted]

SEQ ID NO: 13 - protein, 198 aa, Pan troglodytes
MDSLLMNRKKFLYQFKNVRWAKGRRETYLCYVVKRRDSATSFSLDFGYLRNKNGCHVELLFLRYISDWDLDPGRCYRVTWFTSWSPCYDCARHVADFLRGNPNLSLRIFTARLYFCEDRKAEPEGLRRLHRAGVQIAIMTFKDYFYCWNTFVENHERTFKAWEGLHENSVRLSRQLRRILLPLYEVDDLRDAFRTLGL

SEQ ID NO: 14 - protein, 199 aa, Bos Taurus
MDSLLKKQRQFLYQFKNVRWAKGRHETYLCYVVKRRDSPTSFSLDFGHLRNKAGCHVELLFLRYISDWDLDPGRCYRVTWFTSWSPCYDCARHVADFLRGYPNLSLRIFTARLYFCDKERKAEPEGLRRLHRAGVQIAIMTFKDYFYCWNTFVENHERTFKAWEGLHENSVRLSRQLRRILLPLYEVDDLRDAFRTLGL

SEQ ID NO: 15 - protein, 198 aa, Canis lupus
MDSLLMKQRKFLYHFKNVRWAKGRHETYLCYVVKRRDSATSFSLDFGHLRNKSGCHVELLFLRYISDWDLDPGRCYRVTWFTSWSPCYDCARHVADFLRGYPNLSLRIFAARLYFCEDRKAEPEGLRRLHRAGVQIAIMTFKDYFYCWNTFVENREKTFKAWEGLHENSVRLSRQLRRILLPLYEVDDLRDAFRTLGL

SEQ ID NO: 16 - protein, 206 aa, Oryctolagus cuniculus
MPQTRSSPLVLLLMKQKKFLYHFKNVRWAKGRHETYLCYVVKRRDSATSFSLDFGYLRNTNGCHVELLFLRYISDWDLDPGRCYRVTWFTSWSPCYDCARHVADFLRGNPNLTLRIFTARLYFCEDRKAEPEGLRRLHQAGVQLGIMTFKDYFYCWNTFVENRERTFKAWEGLHENSVRLSRQLRRILLPLYEVDDLRDAFRTLGL

SEQ ID NO: 17 - protein, 198 aa, Rattus norvegicus
MDSLLMKQKKFLYHFKNVRWAKGRHETYLCYVVKRRDSATSFSLDFGHLRNKSGCHVELLFLRYISDWDLDPGRCYRVTWFTSWSPCYDCARHVAEFLRWNPNLSLRIFTARLYFCEDRKAEPEGLRRLHRAGVQIGIMTFKDYFYCWNTFVENHERTFKAWEGLHENSVRLTRQLRRILLPLYEVDDLRDAFRILGL

SEQ ID NO: 18 - protein, 198 aa, Mus musculus
MDSLLMKQKKFLYHFKNVRWAKGRHETYLCYVVKRRDSATSCSLDFGHLRNKSGCHVELLFLRYISDWDLDPGRCYRVTWFTSWSPCYDCARHVAEFLRWNPNLSLRIFTARLYFCEDRKAEPEGLRRLHRAGVQIGIMTFKDYFYCWNTFVENRERTFKAWEGLHENSVRLTRQLRRILLPLYEVDDLRDAFRMLGF

SEQ ID NO: 19 - protein, 198 aa, Gallus gallus
MDSLLMKRKLFLYNFKNLRWAKGRRETYLCYVVKRRDSATSCSLDFGYLRNKMGCHVEVLFLRYISAWDLDPGRCYRITWFTSWSPCYDCARHVADFLRAYPNLTLRIFTARLYFCEDRKAEPEGLRRLHRAGAQIAIMTFKDFFYCWNTFVENREKTFKAWEGLHENSVHLSRKLRRILLPLYEVDDLRDAFKTLGL

SEQ ID NO: 20 - protein, 201 aa, Xenopus laevis
MTMDSMLLKRNKFIYHYKNLRWARGRHETYLCYIVKRRYSSVSCALDFGYLRNRNGCHAEMLFLRYLSIWVGHDPHRNYRVTWFSSWSPCYDCAKRTLEFLKGHPNFSLRIFSARLYFCEERNAEPEGLRKLQKAGVRLSVMSYKDYFYCWNTFVETRESGFEAWDGLHENSVRLARKLRRILQPPYDMEDLREVFVLLGL

SEQ ID NO: 21 - protein, 209 aa, Ictalurus punctatus
MSKLDSVLLTQRKFIYHYKNVRWARGRNETYLCFVVKKRNSPDSLSFDFGHLRNRSGCHVELLFLSYLGVLCPGFLGSGVDGVRVAYAITWFCSWSPCSNCAHRLSRFMSQMPNLRLRIFVSRLYFCDEEDSQEREGLRCLQRAGVQVTVMTYKDFFYCWQTFVAQNQKAFKAWDDLHQNSIRLSRKLQRILQPSESEDLRDGFALLGL

SEQ ID NO: 22 - protein, 210 aa, Danio rerio
MICKLDSVLMTQKKFIFHYKNVRWARGRHETYLCFVVKRRIGPDSLSFDFGHLRNRSGCHVELLFLRHLGALCPGLSASSVDGARLCYSVTWFCSWSPCSKCAQQLAHFLSQTPNLRLRIFVSRLYFCDEEDSVEREGLRHLKRAGVQISVMTYKDFFYCWQTFVARRERSFKAWDGLHENSVRLVRKLNRILQPCETEDLRDVFALLGL

SEQ ID NO: 23 - DNA, 18372 bp, Mus musculus
caagagctagggacgcatccaaaagaaccggcaaccgggcccaacagacgttcattttcctgtttgttacatcatccacggtaagcaaggacagcgacagctcaagtcttcaccagaagatgaaggtatcaacaaaacacagtaagggatggaagtctgatacgtggtctaatgtgggcagcttttgaagacgttgggcaaaagtagcccgcagcacagcaagcgcaaggccaatttgtactacatatggaatctttcttgaaaccaaacaaagaacaatcaagaaaaaggaagaaaggatggaagggagggagggagggaaagagacaggaagcaaacacatcgggacaggcgactttggcttccagttatcttgaacaggctaattcacagaaacagaaggtaggttagaggtcgctagggtctggggagtaagggacagctagtgactgttatgtaggtgtatttatttactgattgattgactgatgcaatgaaagttttagggtagggtctggagagatggctcagtgtctaagagcacatattctgtagaggactctggttcagttcccagcacccacaccaggcagcccacaactgcctgtaccaccagagaacataacacaccagtcctccaggcacacacacacacacacacacacacacacacacacacacacacacacacacacgcgcgcgcgcgcacacacatgcatgcacgcacgcacacacacacacacattctttaaggtttttttgtttgttttggttttttgttgtctttctttttgctttatgttttgtttgtttgtttatttgtttgtttttgagaccggctctatgtcctggaattttccatgtagacgaggctggcttgaactcacagagatctgcctacctatgcctcctgaatggtagaattaaaggtgccactacatttcgctctaaaattaaaatttaaaaataaaagttttagggtgggtgagatggttctggaagtaaaggcatttgccaccatcctggaaccccggtgatggaaggcaaaaacggacttctgaaatttgtcctctgacctccacacacacactaaataaatataaaatttacaaattggtttaaattttagaaacaaacagacctgctacgcaagcatgcattctgagtactcagaaggcagaggcaagaggagccggaactcagccccctgacttgtctctgccccacaaaaggatgagaaaggtttaggttccgagtgtaaccattgccacagaatcctgcacttaagcaaagaaacaagcaagcaaacaaacaaacagaaacgccacagacaaacagaagataagcatcaacaatacgctgcttttctccggtccaaaaggccccagtttgcctagagagaccacgcagagcctgcgcagccacattcagagcaagccgcagtggtgtggaacctctccttgaagacgagaaaacatttcctttctttatttctatgttttgttttttgtttttgttttttagcagggttccatgattgtcctggaactggacacatagcccaggctagtctcaaacttccaggaatcctcctgccttaatcttcagaatgctagaattctgatcgtgtacgactgccatacttgtcttgggggcgggattgcctgttccgctgtctgtctggcgacagggtttcactatgtagcccttggttggtctggaatacctttccttctttcttccttaaacatttgaaagatttatttattttatgtatgtgagtacaccgtagttgtcttcagaagcaccagaagagggcatcagatcacaataaagatggttgtgagccaccatgtggttgctgggaattgaacgcaggacctctggaagagcagtcagtgctcttaaccactgagccatctctccagctcgcccactggcttccttgcttgctttttcttaagttttatttatttatttatttatttatttatttatttagttatttagttatttatttagttatgtatattggtattttttcgaagaggacatcagatttcatcttagacggttgtgagccaccatgtgaatgcagagaattgaacccaggtcctctgaaagaacagccagtgctcttaaatactgaaccatctctccagcccctgctgctccctgtccctctcccttttaaagaaatggtgtcagtcagaacaggcaagatggttccatggataaatgtccttgctgcaaagcctgactacttgagttcaagcctcaggatctacatggtggacagagaggaccaagtcttgtaaattgtcttctgacatctacacataagctctggccctcgtgcctcatatacccctccactgccaagcacagcaatatatatataattttttaaaatgtaaagaaatcacaacatctctgccaatatccatcaagtcggccctttgggaggctgtgtacgtgtgtctcagtatgtcattccctggacaattggccaaagtagggcaaaggtccgggcctcatcctgtgagacaagttagagggacttgtccacccaccacctgggttcccttaaccctgtaatgtcacggctggtgctggttactcccggtgccctgaaatttttttcccaggaattcattaattcactagtgagggaaattgtgtctctgatagtgatgtgataatgcagaggaaattaattagaggaagaaggaggatgggggctcattaacatttcagatatgatatccagggaaggctaaactgccagggagtaagccaagtcctgaactatgagactttgcacagagagatttcacagcaacaaaataggggcaggggcatgtgctgtgtgcatgcaacgggatccagtctctagctcaagactggtctggtctatatagaaagttccagaccagccagaggagctacataatgacaccctatctaaaaaaaggaagggaaggaaggaaggaaggaaggaaggaagaaaggaaggaaggagggagggagggagggagggagggagggagggaggaaagaaggaaggaaggaaggaaggaaggaaggaaggaaggaaggaaggaaggaaggaaggaagagtataagaaaggaaggaaggaagaaagcaaaggatgttcttccagatgatcagggttcagatcccagcaaccacacatggtggctttcaaccgcctgctggtcctctgcaatagaaataagtgctcttaatcactgggccagctctccaggcctccagtaaggtatttttaatgaggaaaaagagttcttttttaaaaaaaaaatactttttgacacacacacacacacaaaattaaaataaatcactttttggtgcaagcaactagtctttctagctatcttataatgtcattttaaaaaaagaaaaatatattagagaattaggaggctaaagttcactctctggatgctgtggtggtcaacccccatctctactgaggcataaaactgagtgtaacaaacggaaggaacagatactgtaagttcaagaagcacaagatgcatttaaggccactttaagtcactatgactgctatcattcttgttatcacaattttaaaattaggaagcatgcacagaccttaggtgtgatacctgggacccccccacacacacacacacacacactcacagagctcattatcatgataccaatgtgaaaagtgtccagtgctattgtctcctgatctttgttacctgtggtacctgggctggctttttagaggaacagcctcgaaggaagttggacattaagcatgagcagaactgccccccgccccccaatcatttaatccgtgtggctctgcccaccacagccccgcccatctttactggacccaacccaggaggcagatgttggatacctggtggtagtgatgctgtcgtgggggaggagcccacaagagcaagctcagatttgaatgccaggggccagtgctctgtcacacaacagcactgaagcagccttgcttgaagcaagcttcctttggcctaagactttgagggagtcaagaaagtcacgctggagaccgatatggacaggtaacaagacagtctcatctgcttgtgcatgtgctccagtagtgctggctgcctgtctggatggaggctttgcctgtcagtgcgcgaatttcctcgtctgcttgccaccctctgctcaggtcttttgggttttggacctaactctgaccacgaagttcttcccttcccccggtttctctcttctctgtgttgctagagataggaagccttgacttgtcctgagatttgggcagagctagagccggcttgtggtaataacagcgaagccttagaggcccgcgccacaaagaggtcgtagcaactccttactaaaaacagtagtggttattttcacaattatttggcaaatatccaacatcttaagactcgcatggggagtctttacaggaattatttagttatagcaagaagatttgtacttctcaaaaaaaaaaaaaaaaaaaaaaaaactaaacatttgagatgaattgcttgcaactcattacaatggtgtctattgaaggagagaatttcattaagacaggcaatttagtgttatagactcaactgttagacacttggtgacatttttactgtttaattcatctatgcagagatttcttagcttcttgaaagcttttatatgcagctcatgatgagccattatcagaaatttctctcttgatttttacatttattgccagtgtgtgagtcactatgcctaaagcccatacacttgagctcacttccgtttggctatgaggtttagaatatggagttaatatagctaatggtagcagggtgttcttcagattccagatttttcctttcttgtcttccttctttctttttgttacccttctcctaccccctcttcttctccccctcctcctcttcctccctcctccccatctcttcccctcctcttctccctcctccccctcctcttccccttcccctcttccccctcttcctcctcttcctcctccttctcctgctccccatctctttccctcctcttctccctcctcctcctccttttgccccttctcctcttccccctcctcctcttccttttccttctcctccttctcctcctccctctcctccccctttcctcctcccactctccctcacccctatcagggaccacattgaagacctcacacatgctagacaagtaatctgccacttaattacatcctgagccctcaaaaagcaaacagacagacagacagacagacaaacaaacaaacaaacaaacaaatgttcacaggaggcaggcagacagcatgagctgcttctgggtttatagtgaattttgaaaccaaatctgagatctatgtcctgatggagaagggtccgagagaaatgcatgagcatggcaaaatgcaaagcaaagacgaggctgagattcagggagaagcaaacaagacagtggagagacacaggatggcacggcatggactggagcaagggcagcgggtaactcaaggcagccctgctactaggctgggattatttttaaccccttgagtctggtttgcattgctggggaagcagctaaggttctgcctcaaggagcacagctgtctcagcagctggcgatctacaggtttgggacaccacctagcaaagtcctccttaccgggagggacatcccgaggagagggagctggaaataggctcctagctagagttgaggggagtgctggatggaggtgcccagtccacaggtcaggactgtgcagctcctcccaccgtggctggaatcttaaaatagaaacagtctattacatcttcctgtggttcagacacaactcttctatttgagacacatcctttctaaactccaaggatacctttccttcataatttcagcatccacccccaatacacactcataaatacacaaacacacacacagagtaagagagagagaaagagagagataagcacgtatgtacacttgctacccacagtatgtaggaaaagttctctagggctgtgtgtacggctctgtggcacagcactcactagcaggtacaagactccatgttcaacccactgaaaaagattctctacttttcccatctaggtaacacaggaagtttagttaaatagaaagggaatttattgctaagagatgaagtttaagctgtttaaaactggctggattagagagatacctgtgcttattattataacatgctgagtttacctgtactgtggtggtgatgatgataatgatgctgtgtcatcacatagcccccgtggcttagaattctccatgaaagtcaggctggcttccattacagaaagatccacctgcctctgccccccttcgcccccaagttctggatttaaaggtgtgcacaccatgcccagcttctaaagggtttttataatttagtgatgaatgtagacatggaggtactatgatcgttatcatggtaaattactatttcaaaataaagctatgatcattagaggccaagacaggaggaccatgagttcgaggccagctgcagcaacatagagatttccagactagcatggatcccgcagcatgagcatgtccccaaaacaattttgtttttccaaaagtcagggactgtcacgtgtgttgaactatcattaaagcatgagctgtgaacgtgtgaacatgcattcaatgatagtatatggttatttatagtggctctaaccactgcagcaccaaagcggaacatatccaaatttcaatcagcacataaatgaataaacaaaacatgttctacccatacaatagaatattgctcggcaggaataaggagccaacttctgatatttgggtgaatataaaatttactatgttccgtgagagcagttacacaggaaggaggaaacgtgatttatatgaaattatagaaagttagaaataatttacatttacagagagcaggtcggcggttgcctgggtaagaggagaaagaacagccaatagcgacatagaagctttaagaagcctagaaatgtctcctctgatggccctggcagtctgggctgcggacctgccggcattcacggagctgtagattttaagcgagtggagctcactatgtaaattgtatctcaacaacaacaaaagtgaaaaacggtttcaattctcttgcatcaaaaccgtattcaaattcctaactagctcttaaaaaaaaaatcattgcacttccatccatcaccactgtgtggcggtgctgtgtcgacaagtgagcgacacagttgtttatcatccgttttatctcctggctcatgtccaccgctttaacaggaactgtaatttttttttttttttaagaaacgtgagggctgggaatatggttctgtgggaacagcgcttgccatgaaagcaaaaggacctgagttgaggggtccaaaagcagacacctgtaattcccgtgcttctaaggcaacataagaggtggagaaaggagaaccccaggaagcttatgagccagttagcccagggcgcacagcagagagcaagagattctatctcaaacaagacagaagtcaaggaccaacacccaaggttgtactctgctcgccacacgtatcctgtagtatgtgttgcctctacccccaccacatacacacacacgcacacactccacaaagattttaaaaattatttttaagtgtgtgtgtgtgtgtgtgtgtgtgtgtgtgtgtgtgtgtgtgtcaatgcatgtgccctcaaaagtcagaggtgtcggatcctggtggaactggagtgacaggtggttgtgagctgcctgatggaagagcagttcgtgctcataactgctgagccatctctctagtccccagaaaacctgtatttttaaagaaagaaagaacgaacgagaaaaaaaaaacctcgggggctggagatgtagctctactagagaacttgccagcatgcacaaagccctgggttaagtccccaacaagtaggtgggacaagcctgttgtcccaagaccaggggagtagaggaggcaggagagttctccttgtcaaattccccaccagcctgagctaaatgagttctcatctcaaaacaaacaataaaaataaataaataaaataaaatcaacaaaactgacccagcaaaatccaaaaatgaaaacccaaagacctaagcaggggtgggggttaggggggcgtggtttgtcagacggctcagcaggtaaaggcataaactgccaaacctgatgtcttgagtttgatccctcgagctcacatgatggaagcagagagccaacttccacaagttgccctttgacctccacaggtatgtgtatgtccctacacacatatcattcatacaacgatagtaaacaaatgtgattttttttaaagacacagaggcaactatttcttatacttctgtttaatgaagaatggatagatactatgtagcaacgtgatcgcaagtcgacatgacctcatatgaccctgtgcttagagagggaggaaggacgcccccagcaaggcccatctgcaacattccttttcctggatagagacaggacacccacagaatatggctcttcaaggaagagagtgacctttcttttcgcaggagctcagtggctttgataccctgttgtcttccttcctcgctgtggctcaagtgctggaagtagagagtgactttctatgttttcctttgctttgtcttgtactgagtcagacctagagcctcatacatgataggcaactgctgagctactgagctacgttcttgtggtggagctcaggctgggctcaaacttacagcaacctccctcccccagccttccccccactcccccccgccacccccccaccccccacccccgcactcccaagtgctgaggttacaggcacaagcaacctaacctggcccttgccatgttttataacttgcttttggaagacttctggttctgtgatgctactgggttagcggggagacaggagggcagaaggttaaaggtgtctaagaccatgtccaaagcccagtaggaggattagggagatggctgggctggagagatggctcagtggttaagagcactggctgtgtttgcaggggaccagagttcaattcccagcaaccacataatggctcacaatcatctacaatgggatctgacaccctcctttgacatgcaggcatgcatgtacacagagcagtcatacataaattatataaataaatacattaaaaaataaaaagtaataaagggtaattacctagtttgactgttgcagcgaggggggaggggaagaggaaagggaagagggtgggcagggaaggattttaaagtgagcatgtctcaggtatccagaaaaggaagcacgactatgctttctggtttaacctatataatgataagatttaaaacatcatgatgatcaaagtaggcctggggatgcagctcggtgctgaagcgctcagctaccttgcctatggctgtaggtccagccatcagcagctgcaacaataataatcagagctgaaggaagactatggtgacagagaagccttgccctgactttcttctccaactcacagccttctgatgaagcaaaagaagtttctttaccatttcaaaaatgtccgctgggccaagggacggcatgagacctacctctgctacgtggtgaagaggagagatagtgccacctcctgctcactggacttcggccaccttcgcaacaaggtggggggggggttgcggggttggggagggggtgggtggcagctgttgacactaaagctatggctgcccctggtgccaaatgttgaggggaccaaggcaggccgattgctgagtttgagacagcctggtctacagagagagttttaggactacacagagaaaccctgtctggaaaaacaaacaaacaagcaaacaaagagtgaaataatggtgcatgcctgtatttccacagtgctagggctgaaatgaaggatctgccttgccagacaagccccgcccctgagccctcccctaaccgcctctggcccctcagcccctcagccctttaatccctcagctctgggttctttctcaagcactttcttgagtgagaaaaacaaattatatcttcagaatttttgaaaatcaatgaggaaaaaaataggtaaaatgacatcaactcaactttatttcccaaacaattttgttcccaaaagaccagagaggccaatgaccgaccacctttaacccaatgagtttccttcagggaccagagagaagtctctgttgtttgggtaattagataatccttcggctgcctgaaagaactgcgtttctaagagagttcaccaaattgcagattggcttccatgggcttctccttctctacttggagtcatgacacactgtatttatagacagcttgatcaagtggtactttctcttcgcacacaacaccagcttgatttactgctaaggaaatagtgcaaaaaaagatgagtaaaagaaaaactatcttcagtcttcgacaaacgattttcgcaataggagatgggcctattacgattgcagttattacagtcactggcatcacatagcatgtacacacacgcgcgcgcgcgcgcgcacacacacacacacacacacacacacacacacacacacacacacaccccttaattgccttccacttaaaacgccagacgccaagtcagagacgaaatctcttcaataagctttttcctccctccttacaaattattctggcgccacctagtggccaaggtgcagtttgcagttttacaacgtggcgtccaaacaggcacttccgggacacgaaggtaatccctgcaaggtgtgtatcctttgtcccatagatgtgcagctttcctttacccaacaaagccagtgtaataaagccatttgactccaacaagtgctatcttaataagagaattatctttatgctgggagtgatggcacacacctttaatcccaaccctccagaggcagaggcagatggatctctgtgagtttgaggactgcctggtctacataatgagttccaggtcaagccagtgcgacatccccacaagcatcccaaatggcctgggtgggagagcatgcaggtcacgtcaccagtgctctctgctctttctccagtctggctgccacgtggaattgttgttcctacgctacatctcagactgggacctggacccgggccggtgttaccgcgtcacctggttcacctcctggagcccgtgctatgactgtgcccggcacgtggctgagtttctgagatggaaccctaacctcagcctgaggattttcaccgcgcgcctctacttctgtgaagaccgcaaggctgagcctgaggggctgcggagactgcaccgcgctggggtccagatcgggatcatgaccttcaaaggtgagacttgcacactggagagagcggtctgagttgccactcagagtgagtgtcagcggggaaactgggggtggggtgctacttaaagaccttcagttcgtcctggatatcaaaagtattactttattttttgaggtaggatctcgctatcccaggctgaccttcaacttgcaattctccgacctctgccttctgagtggcggaattacaagtatacatcaatctcagaattatcagaatttgagagatagaagttggcagggctacaggtgcgctcagtggcagaactctggtccagcatgtgcaaagccctgcattccacctttagcagtcaaataataaattgaggagggagaggaggaggatagtggtcagagagatggttccgtgggggcccttgcctttgtaccttaagtttaacccctaaaacactctgactttctgaccttcacctacacacacacacacacacacacacacacacacacacacacctccttcttatttatctatttatttttcttttaagactatttttactgctggaatacatttgtagaaaatcgtgaaagaactttcaaagcctgggaagggctacatgaaaattctgtccggctaaccagacaacttcggcgcatccttttggtaagtctgcctgtctgtctgcctgtctctctgtctgtctctgtctctctgtctgtctctgtctctctctctctctctcatacacacacacatacatacactcacacacacacacacacacctggagcctcttagttatttgtttgtattatgcattattttatacaatgattacttcaaggcacttacaacccagttttcttttctgctttacccaggacagagcttccacttagacgcttgcctcttgcctcctcttcgctcagtcttcataactctttccttttgctaacctcccctcaggtggggttccttccagggcagaattcgccccttctttttttcctggtcctcaagcaatttactttcctctggagccacccacttcgtttagacactttcctttccagagatcaaatttaaagcccttcactccgtttatatcatctctctttctccacagcccttgtacgaagtcgatgacttgcgagatgcatttcgtatgttgggattttgaaagcaacctcctggaatgtcacacgtgatgaaatttctctgaagagactggatagaaaaacaacccttcaactacatgtttttcttcttaagtactcacttttataagtgtagggggaaattatatgactttttaaaaaatacttgagctgcacaggaccgccagagcaatgatgtaactgagcttgctgtgcaacatcgccatctactggggaacagcagaacttccagactttgggtcgtgaatgatgctcttttttttcaacagcatggaaaagcatatggagacgaccacacagtttgttacacccaccctgtgttccttgattcatttgaattctcaggggtatcagtgacggattcttctattctttccctctaaggctcactttcaggggtccttttctgacaaggtcacggggctgtcctacagtctctgtctgagcaatcacaagccattctctcaaaagcattaatactcaggcacatgctgtatgttttcactgtccgtcgtgtttttcacatttgtatgtgaaagggcttggggtgggatttgaagaatgcacgatcgcctctgggtgatttcaataaaggatcttaaaatgcagatgaggactacgaagaaatcactctgaaaatgagttcacgcctcaagaagcaaatcccctggaaacacagactctttttcatttttaatgtcattagtttactcacagtcttatcaagaagaagagttcaagggttcaacccaattttcagatcgcgtcccttaaacatcagtaattctgttaaagggatcaaacatccttatttcttaactaactggtgccttgctgtagagaaaggagcaaagcgcccagatccaaagtatatagttatcatagccaggaaccgctactcgttttccattacaaatggcaaattcttccccgggctctcctcatagtgcctgagacggaccacggaggtgatgaacctccggattctctggcccaacacggtggaagctctgcaagggcgcagagacagaatgcggcagaaattgcccccgagtcccaactctcctttccttgcgaccttgggaacaagacttaaaggagcctgtgacttagaaacttctagtaatgggtacctgggagtcgtttgagtatggggcagtgatttattctctgtgatggatgccaacacggttaaacagaatttttagtttttatatgtgtgtgatgctgctcccccaaattgttaactgtgtaagagggtggcaaaatagggaaagtggcattcacctatagttccagcattcaggaagctgaggcaggaggattgtaaatttgaggccagtctgagctgtaaggtgagaccctatttcaaacaacacagccagaattgggttctggtaaatcatacttaacaagggaaaaatgcaagacgcaagaccgtggcaaggaaatgacgctttgcccaacgaaatgtaggaaaccaacatagactcccagtttgtccctctttatgtctggtctccctaacaacgatctttgctaatgagaaaaatattagaaaaaaatatccctgtgcaattatcacccagtcgccattataatgcaattaaaaggcccacaagaaatcctgtatacacgaccgttatttattgtatgtaagttgctgaggaagaggagaaaaaaataaagatcatccattccttcctgcatctatccctgttttttatgttgctgcgtggcatctattctgaaatattaaagtgggtgcctgaagtttcataaatttgaaactttagagattactatatatctgcactcgtcattgtgatcatccaaaatcgtaatgattatggctcggcagctgtgctcttgatttttagcaactcccacccccacccccacccccacccccacccccaacccccaccctgcgtgcagcaagttcatcctggcttattttaaatcaactgaattcgagattaaaatgtgaaagttttggagatgaactactgaataaaatgatgtcgggaaaaagcatttatatattaaagtcatacagatcacagggaaggtggcgcatgtatttaacccccagcattggaaagatggaggcaggaggctctctgtgggtttgaggtcagcctgatctagacagagtgctccgcagatagccacacagagagagcctgccttagagaaataaatacctgatgaaatagaattgaattgagagtccagaaattaacccactcagctatgaccaactgatttcagataaaggtccaaggtgtactgagtcagcaaaccctgctggggcaatctgacatcaggtgcaaagagtgcagtccacatggtgacacctgcctgtcccctactcgggaggctgagacaagaagatcagtagtagttgcagtaaatctccacccaaatatgccctggcaatgaaaacacaactcaattaatatgaatacatgctgtgcgcctagattgggcagatctaccgctgcactaccatcttctccatctatgagaccctttagaacttgcggtttctaaggtttgggggtataattagccccagggctatccacaacactgtcctaggcgcatttcctaaacacgagcttattcataagcccagccagagggttcacattgcccacaacacaccctcctttcctaccacataaccaaagcccaaactctagaactggttctaactgggaattctcatggcatcccatagcatatacccccttctctgcagtgagcaatatgtccagtatttcctggaaaccattggtacacaaaactctgagtcaccaacacccgctgctctgtctactgaactggcttccaatgttaactaattcatttgagtgtgtgtattagtgtgagtgtgtgttagttacttttgctgttgttgtgattaaaacaccatgaccaagggcaacctaaggaagagagcgtgtgtatcttggcttatgccactgagaacgaagccatcactgtggggtggaggcatggcttcaagtgtcaggcatgacgtgaggagcaggaagctgggagatcacatctttaacagcgagtgccaagcagagagggaaactggaagttaaagcccacaagtgatgtgctccctcagccaggctgcacttcctgaacaccccaaaacagcgccacctacctagaaccgactttgaatatctgagcctatggggacatttgtcattctaaccactgttgtgcactgttgttgcacagtgagccatcttgccagctcatattccacaatttgtatttcattttaccaatgctctctctgtagtagtgataatgatgactgttcccttttttggttttgcttcgttttgagatttcagtatttttctcaagttttattttaagtgatgttaattacagcgtttgaaggggaggagctaattccactcaaaatggaagactctataatgtacccattaaactgctaaaaaaaaaataataataataatggtaagtctacaagaggagtcagtttagacccctagtgttgtcagagtgtgaccacaatcacctgcccagatcagagccagagaacccggaagctatttcatactctggtgcaatggggggggggggggggggagaaattttaaaaaaacaaaaaggaggaagaaaaacacacacacaacacaaggaagaattaagtcctgattgactgactccatcttgcccaccctctccaccctaaaatggcacaaaagaaaataccacacctaaagactacttttggtgtaaaacaggtaactgatgggctaggatgggaacagggtatgatgatctgtctaaaaaaatgttcctttcacgaaggtgtgtacgtacttctgagcagataggatcgggacaccagggttcaatgcttgggaagtcacaatttcatctggggactggatacagatttacaaagggtccacacattcccagcttccatttgcagcctggcatctctagaggctcctccccaagccccaacccacacctacagctagaaaggaccctttctggaatggggtttctgctgtacctctgaaatggtaaacaccttaaagctgagtcatccttagcctggagaggcattcatcaactctcgcatccccaacatacaatattaaaagtccactaaattggtagctatgttgcaaaatagttcaaaattaacgattttacaatattcatttatgcttgaaattctagtcctaagccaagcttgtgtctgccagcattgatgttcttgcgtccagtagggctgacaatgtcagtttgatacctggttttaggatctgagtgtaccctaagccaatcaggctggagttgttcactttgccagaaaagcaggcatcagggtggaactgaaatttggctgctattccaaagcgagtgttactgttttctgcagtccaggcgagattgacagcagtctccaacttcttgttcgccttctggtaaatggaaccaccaaactctgtcccgtcgtatgaagctgtcttgctctgggtcactcaggacttcgaggtctcaaaattcatctggtagccagctagccaaccctcatagccaagcaccggattgagggcccagcgatgtcaaagtccacgccacagcccaagttgatgtgctccctcttgtaccctgtcatgatttttagcattttcccccaagttttgggtgaaaaagatgaatcgaaggtcagcttcagtccacaagcaagctggtctcccacggtgatctcagtgcccagggtgctgtctgtgttccacttctccgtaaatgtttgcccatactcagtccatctgtccttggtttccagactgccgttcactctggtggtctccgtgttggcagagcctgagctggtaaattccaatctattcttggacttcgttttcaaatcaagttttattaagccaaagcggtagcccttggtgaagacatccctgatggatagaggcagctgcgatggggggttgcagcgaggatgctgggagcgcagcgaataggcagagggcggggcagctctcacgattgtttcttaagaagacttcctttaaaattaatactaatccactaactactcactcattcttccaggattttactgatcaattgctgtatacgcatagcgccgcggtcatcgttacacagacgtgttaagcacacaaagactgctttgaagaaggctgaaagatctcggggctggagagagaactctgcagtttacagagcttcttggtcctccagagaacccaatttcagttcccagcatccacatcacacagctcacaaccgccggaaactccagctccagagggtccaacacccctgttctggcctccaggagcacctacatacatgtgtcatgcaaacacacacacaaacacacaacacacacatacatacataaattaaaaatatatataaataaatcaatcctttttttttaaagcagtcttaaaatctgtggacctagagaagtattatctgaaattttgaaatgggacccaaagaacgtcttctcacaggaactaatacttacagtcttttgaagcataggtaaatgttcaatcggtgatgataaacctagagactgagactgcagccaggctgggagaggacttgtccagcatgcgctaagtccagtgctcagcccac

SEQ ID NO: 24 - DNA, 96 bp, Artificial Sequence
  Primer 1
acaataataatcagagctgaaggaagactatggtgacagagaagccttgccctgactttcttctccaactcacagctgtgacggaagatcacttcg

SEQ ID NO: 25 - DNA, 96 bp, Artificial Sequence
  Primer 2
caccaggggcagccatagctttagtgtcaacagctgccacccaccccctccccaaccccgcaaccccccccccacctgaggttcttatggctcttg

SEQ ID NO: 26 - DNA, 96 bp, Artificial Sequence
  Primer 3
cccacaagcatcccaaatggcctgggtgggagagcatgcaggtcacgtcaccagtgctctctgctctttctccagctgtgacggaagatcacttcg

SEQ ID NO: 27 - DNA, 96 bp, Artificial Sequence
  Primer 4
cccacccccagtttccccgctgacactcactctgagtggcaactcagaccgctctctccagtgtgcaagtctcacctgaggttcttatggctcttg

SEQ ID NO: 28 - DNA, 96 bp, Artificial Sequence
  Primer 5
acacacacacacacacacacacacacacacacacacacacctccttcttatttatctatttatttttcttttaagctgtgacggaagatcacttcg

SEQ ID NO: 29 - DNA, 96 bp, Artificial Sequence
  Primer 6
gagagagagagagacagagacagacagagagacagagacagacagagagacaggcagacagacaggcagacttacctgaggttcttatggctcttg

SEQ ID NO: 30 - DNA, 96 bp, Artificial Sequence
  Primer 7
gtttagacactttcctttccagagatcaaatttaaagcccttcactccgtttatatcatctctctttctccacagctgtgacggaagatcacttcg

SEQ ID NO: 31 - DNA, 96 bp, Artificial Sequence
  Primer 8
ccagtagatggcgatgttgcacagcaagctcagttacatcattgctctggcggtcctgtgcagctcaagtattttctgaggttcttatggctcttg

SEQ ID NO: 32 - DNA, 107 bp, Artificial Sequence
  Primer 9
cccacaagcatcccaaatggcctgggtgggagagcatgcaggtcacgtcaccagtgctctctgctctttctccagaacggctgccacgctgagatgctcttcctgcg

SEQ ID NO: 33 - DNA, 99 bp, Artificial Sequence
  Primer 10
cccacccccagtttccccgctgacactcactctgagtggcaactcagaccgctctctccagtgtgcaagtctcacctttgtagctcatgacagacagtc

SEQ ID NO: 34 - DNA, 75 bp, Artificial Sequence
  Primer 11
aggcaaagcctccatccagacaggcagccagcactactggagcacatgcacaagcagatgagactgtcttgttac

SEQ ID NO: 35 - DNA, 94 bp, Artificial Sequence
  Primer 12
gtttagacactttcctttccagagatcaaatttaaagcccttcactccgtttatatcatctctctttctccacagccgccgtacgacatggagg

SEQ ID NO: 36 - DNA, 102 bp, Artificial Sequence
  Primer 13
aggcaaagcctccatccagacaggcagccagcactactggagcacatgcacaagcagatgagactgtcttgttacttaaagcccaagtagaacaaacacttc

SEQ ID NO: 37 - DNA, 96 bp, Artificial Sequence
  Primer 14
tatgactgtgcccggcacgtggctgagtttctgagatggaaccctaacctcagcctgaggattttcaccgcgcgcctgtgacggaagatcacttcg

SEQ ID NO: 38 - DNA, 96 bp, Artificial Sequence
  Primer 15
cagtgtgcaagtctcacctttgaaggtcatgatcccgatctggaccccagcgcggtgcagtctccgcagcccctcctgaggttcttatggctcttg

SEQ ID NO: 39 - DNA, 95 bp, Artificial Sequence
  Primer 16
tatgactgtgcccggcacgtggctgagtttctgagatggaaccctaacctcagcctgaggattttcaccgcgcgcctctatttctgcgaggagcg

SEQ ID NO: 40 - DNA, 78 bp, Artificial Sequence
  Primer 17
ctttgaaggtcatgatcccgatctggaccccagcgcggtgcagtctccgcagcccctccggctccgcgttgcgctcct

SEQ ID NO: 41 - DNA, 33 bp, Homo sapiens
ctctacttctgtgaggaccgcaaggctgagccc

SEQ ID NO: 42 - DNA, 33 bp, Mus musculus
ctctacttctgtgaagaccgcaaggctgagcct

SEQ ID NO: 43 - DNA, 33 bp, Gallus gallus
ctctacttctgtgaagatcgcaaggctgagcct

SEQ ID NO: 44 - DNA, 33 bp, Xenopus laevis
ctctatttctgcgaggagcgcaacgcggagccg

SEQ ID NO: 45 - DNA, 36 bp, Ictalurus punctatus
ctctacttctgtgacgaggaggacagtcaagagaga

SEQ ID NO: 46 - DNA, 36 bp, Danio rerio
ctgtacttctgtgatgaagaggacagcgtggagaga

SEQ ID NO: 47 - protein, 11 aa, Homo sapiens
LYFCEDRKAEP

SEQ ID NO: 48 - protein, 11 aa, Mus musculus
LYFCEDRKAEP

SEQ ID NO: 49 - protein, 11 aa, Gallus gallus
LYFCEDRKAEP

SEQ ID NO: 50 - protein, 11 aa, Xenopus laevis
LYFCEERNAEP

SEQ ID NO: 51 - protein, 12 aa, Ictalurus punctatus
LYFCDEEDSQER

SEQ ID NO: 52 - protein, 12 aa, Danio rerio
LYFCDEEDSVER

SEQ ID NO: 53 - DNA, 597 bp, Artificial Sequence
  Chimaeric AID (mouse AID with Xenopus exon 3)
atggacagccttctgatgaagcaaaagaagtttctttaccatttcaaaaatgtccgctgggccaagggacggcatgagacctacctctgctacgtggtgaagaggagagatagtgccacctcctgctcactggacttcggccaccttcgcaacaagaacggctgccacgctgagatgctcttcctgcgctacctgtctatatgggtgggtcacgacccccataggaactaccgggtcacgtggttcagctcctggagcccctgctatgactgtgccaagcgcaccctcgagttcttaaaggggcaccccaacttcagtctgcgcatcttcagcgccaggctctatttctgcgaggagcgcaacgcggagccggaggggctgcggaaactgcagaaagcgggggtgcgactgtctgtcatgagctacaactatttttactgctggaatacatttgtagaaaatcgtgaaagaactttcaaagcctgggaagggctacatgaaaattctgtccggctaaccagacaacttcggcgcatccttttgcccttgtacgaagtcgatgacttgcgagatgcatttcgtatgttgggattttga

SEQ ID NO: 54 - protein, 198 aa, Artificial Sequence
  Chimaeric AID (mouse AID with Xenopus exon 3)
MDSLLMKQKKFLYHFKNVRWAKGRHETYLCYVVKRRDSATSCSLDFGHLRNKNGCHAEMLFLRYLSIWVGHDPHRNYRVTWFSSWSPCYDCAKRTLEFLKGHPNFSLRIFSARLYFCEERNAEPEGLRKLQKAGVRLSVMSYNYFYCWNTFVENRERTFKAWEGLHENSVRLTRQLRRILLPLYEVDDLRDAFRMLGF

SEQ ID NO: 55 - DNA, 597 bp, Artificial Sequence
  Chimaeric AID (mouse AID with Xenopus active-site loop)
atggacagccttctgatgaagcaaaagaagtttctttaccatttcaaaaatgtccgctgggccaagggacggcatgagacctacctctgctacgtggtgaagaggagagatagtgccacctcctgctcactggacttcggccaccttcgcaacaagtctggctgccacgtggaattgttgttcctacgctacatctcagactgggacctggacccgggccggtgttaccgcgtcacctggttcacctcctggagcccgtgctatgactgtgcccggcacgtggctgagtttctgagatggaaccctaacctcagcctgaggattttcaccgcgcgcctctatttctgcgaggagcgcaacgcggagccggaggggctgcggagactgcaccgcgctggggtccagatcgggatcatgaccttcaaagactatttttactgctggaatacatttgtagaaaatcgtgaaagaactttcaaagcctgggaagggctacatgaaaattctgtccggctaaccagacaacttcggcgcatccttttgcccttgtacgaagtcgatgacttgcgagatgcatttcgtatgttgggattttga

SEQ ID NO: 56 - protein, 198 aa, Artificial Sequence
  Chimaeric AID (mouse AID with Xenopus active-site loop)
MDSLLMKQKKFLYHFKNVRWAKGRHETYLCYVVKRRDSATSCSLDFGHLRNKSGCHVELLFLRYISDWDLDPGRCYRVTWFTSWSPCYDCARHVAEFLRWNPNLSLRIFTARLYFCEERNAEPEGLRRLHRAGVQIGIMTFKDYFYCWNTFVENRERTFKAWEGLHENSVRLTRQLRRILLPLYEVDDLRDAFRMLGF

SEQ ID NO: 57 - DNA, 600 bp, Artificial Sequence
  Chimaeric AID (mouse AID with catfish active-site loop)
atggacagccttctgatgaagcaaaagaagtttctttaccatttcaaaaatgtccgctgggccaagggacggcatgagacctacctctgctacgtggtgaagaggagagatagtgccacctcctgctcactggacttcggccaccttcgcaacaagtctggctgccacgtggaattgttgttcctacgctacatctcagactgggacctggacccgggccggtgttaccgcgtcacctggttcacctcctggagcccgtgctatgactgtgcccggcacgtggctgagtttctgagatggaaccctaacctcagcctgaggattttcaccgcgcgcctctacttctgtgacgaggaggacagtcaagagagagaggggctgcggagactgcaccgcgctggggtccagatcgggatcatgaccttcaaagactatttttactgctggaatacatttgtagaaaatcgtgaaagaactttcaaagcctgggaagggctacatgaaaattctgtccggctaaccagacaacttcggcgcatccttttgcccttgtacgaagtcgatgacttgcgagatgcatttcgtatgttgggattttga

SEQ ID NO: 58 - protein, 199 aa, Artificial Sequence
  Chimaeric AID (mouse AID with catfish active-site loop)
MDSLLMKQKKFLYHFKNVRWAKGRHETYLCYVVKRRDSATSCSLDFGHLRNKSGCHVELLFLRYISDWDLDPGRCYRVTWFTSWSPCYDCARHVAEFLRWNPNLSLRIFTARLYFCDEEDSQEREGLRRLHRAGVQIGIMTFKDYFYCWNTFVENRERTFKAWEGLHENSVRLTRQLRRILLPLYEVDDLRDAFRMLGF

SEQ ID NO: 59 - DNA, 30 bp, Artificial Sequence
  Primer 18
aggcgaattctccatgaaagtcaggctggc

SEQ ID NO: 60 - DNA, 42 bp, Artificial Sequence
  Primer 19
gttagaatgacgatatcggatccatgctagtctggaaatctc

SEQ ID NO: 61 - DNA, 37 bp, Artificial Sequence
  Primer 20
tggatccgatatcgtcattctaaccactgttgtgcac

SEQ ID NO: 62 - DNA, 33 bp, Artificial Sequence
  Primer 21
aggcacgcgtctaaactgactcctcttgtagac

SEQ ID NO: 63 - DNA, 33 bp, Artificial Sequence; identical to SEQ ID NO: 62
  Primer 22

SEQ ID NO: 64 - DNA, 33 bp, Artificial Sequence; identical to SEQ ID NO: 62
  Primer 23

SEQ ID NO: 65 - DNA, 32 bp, Artificial Sequence
  Primer 24
aggcgaattctttcttagacgtcaggtggcac

SEQ ID NO: 66 - DNA, 30 bp, Artificial Sequence
  Primer 25
aggcacgcgtcgatacgcgagcgaacgtga

SEQ ID NO: 67 - DNA, 75 bp, Mus musculus
tatgactgtgcccggcacgtggctgagtttctgagatggaaccctaacctcagcctgaggattttcaccgcgcgc

SEQ ID NO: 68 - DNA, 58 bp, Mus musculus
gaggggctgcggagactgcaccgcgctggggtccagatcgggatcatgaccttcaaag